SEQUENCE LISTING (48 sequences)

SEQ ID NO: 1 - protein, 107 aa, Mus musculus
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKR

SEQ ID NO: 2 - protein, 107 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 3 - protein, 108 aa, Homo sapiens
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIKR

SEQ ID NO: 4 - protein, 10 aa, Mus musculus
RASSSVSYMH

SEQ ID NO: 5 - protein, 7 aa, Mus musculus
APSNLAS

SEQ ID NO: 6 - protein, 9 aa, Mus musculus
QQWSFNPPT

SEQ ID NO: 7 - protein, 122 aa, Mus musculus
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSS

SEQ ID NO: 8 - protein, 122 aa, Artificial sequence [sequence omitted]

SEQ ID NO: 9 - protein, 119 aa, Artificial Sequence
  sequence is synthesized
EVQLVESGGGLVQPGGSLRLSCAASGFTFSSYAMSWVRQAPGKGLEWVAVISGDGGSTYYADSVKGRFTISRDNSKNTLYLQMNSLRAEDTAVYYCARGRVGYSLYDYWGQGTLVTVSS

SEQ ID NO: 10 - protein, 10 aa, Mus musculus
GYTFTSYNMH

SEQ ID NO: 11 - protein, 17 aa, Mus musculus
AIYPGNGDTSYNQKFKG

SEQ ID NO: 12 - protein, 13 aa, Mus musculus
VVYYSNSYWYFDV

SEQ ID NO: 13 - protein, 213 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 107 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWSFNPPTFGQGTKVEIKR

SEQ ID NO: 17 - protein, 122 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSASYWYFDVWGQGTLVTVSS

SEQ ID NO: 18 - protein, 213 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 107 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWAFNPPTFGQGTKVEIKR

SEQ ID NO: 22 - protein, 122 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSS

SEQ ID NO: 23 - protein, 213 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 107 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 26 - protein, 213 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCAVSNKALPAPIEATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 32 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIELTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 122 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSS

SEQ ID NO: 34 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 36 bp, Artificial sequence
  sequence is synthesized
ctacaccttcacgagctataacatgcactgggtccg

SEQ ID NO: 36 - DNA, 51 bp, Artificial sequence
  sequence is synthesized
gattaatcctgacaacggcgacacgagctataaccagaagttcaagggccg

SEQ ID NO: 37 - DNA, 38 bp, Artificial sequence
  sequence is synthesized
gaatgggttgcagcgatctatcctggcaacggcgacac

SEQ ID NO: 38 - DNA, 65 bp, Artificial sequence
  sequence is synthesized
attattgtgctcgagtggtctactatagcaacagctactggtacttcgacgtctggggtcaagga

SEQ ID NO: 39 - DNA, 36 bp, Artificial sequence
  sequence is synthesized
ctgcacagccagctcttctgtcagctatatgcattg

SEQ ID NO: 40 - DNA, 42 bp, Artificial sequence
  sequence is synthesized
aactactgatttacgctccatcgaacctcgcgtctggagtcc

SEQ ID NO: 41 - DNA, 45 bp, Artificial sequence
  sequence is synthesized
tattactgtcaacagtggagcttcaatccgcccacatttggacag

SEQ ID NO: 42 - DNA, 37 bp, Artificial sequence
  sequence is synthesized
gtttcactataagtgtcgacaagtccaaaaacacatt

SEQ ID NO: 43 - DNA, 33 bp, Artificial sequence
  sequence is synthesized
gccaggatagatggcgccaacccattccaggcc

SEQ ID NO: 44 - DNA, 26 bp, Artificial sequence
  sequence is synthesized
aagctccgaaaccactgatttacgct

SEQ ID NO: 45 - DNA, 45 bp, Artificial sequence
  sequence is synthesized
ccagacgtcgaagtaccagtagcggtagctatagtatacgacgcg

SEQ ID NO: 46 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 47 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 48 - protein, 185 aa, Homo sapiens
MRRGPRSLRGRDAPAPTPCVPAECFDLLVRHCVACGLLRTPRPKPAGAASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

(19) United States
(12) Patent Application Publication
(10) Pub. No.: US 2006/0246004 A1
(43) Pub. Date: Nov. 2, 2006

(54) Antibody variants and uses thereof

(75) Inventors: Camellia W. Adams, San Jose, CA (US); Henry B. Lowman, El Granada, CA (US); Gerald R. Nakamura, San Francisco, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/348,609

(22) Filed: Feb. 6, 2006

Related U.S. Application Data
(60) Provisional application No. 60/702,571, filed on Jul. 25, 2005.
(60) Provisional application No. 60/689,404, filed on Jun. 10, 2005.
(60) Provisional application No. 60/651,111, filed on Feb. 7, 2005.

(51) Int. Cl. A61K 51/00; C07H 21/04; C12P 21/06; A61K 39/395; C12N 5/06; C07K 16/28; C07K 16/46

(52) U.S. Cl. 424/1.49; 424/144.1; 435/69.1; 435/358; 435/320.1; 530/388.22; 530/391.1; 536/23.53

(57) ABSTRACT

The invention provides improved humanized CD20 binding antibodies for treatment of B cell malignancies and autoimmune diseases.

This application claims the benefit of U.S. Provisional Application Ser. No. 60/651,111, filed Feb. 7, 2005, 60/689,404 filed Jun. 10, 2005, and 60/702,571 filed Jul. 25, 2005, which applications are incorporated herein by reference in their entirety.

### FIELD OF THE INVENTION

The invention relates to anti-CD20 antibodies and their use in the treatment of B-cell related diseases.

### BACKGROUND OF THE INVENTION

Lymphocytes are one of several populations of white blood cells; they specifically recognize and respond to foreign antigen. The three major classes of lymphocytes are B lymphocytes (B cells), T lymphocytes (T cells) and natural killer (NK) cells. B lymphocytes are the cells responsible for antibody production and provide humoral immunity. B cells mature within the bone marrow and leave the marrow expressing an antigen-binding antibody on their cell surface. When a naive B cell first encounters the antigen for which its membrane-bound antibody is specific, the cell begins to divide rapidly and its progeny differentiate into memory B cells and effector cells called “plasma cells”. Memory B cells have a longer life span and continue to express membrane-bound antibody with the same specificity as the original parent cell. Plasma cells do not produce membrane-bound antibody but instead produce secreted form of the antibody. Secreted antibodies are the major effector molecules of humoral immunity.

The CD20 antigen (also called human B-lymphocyte-restricted differentiation antigen, Bp35) is a hydrophobic transmembrane protein with a molecular weight of approximately 35 kD located on pre-B and mature B lymphocytes (Valentine et al. J. Biol. Chem. 264(19):11282-11287 (1989); and Einfeld et al. EMBO J. 7(3):711-717 (1988)). The antigen is also expressed on greater than 90% of B cell non-Hodgkin's lymphomas (NHL) (Anderson et al. Blood 63(6):1424-1433 (1984)), but is not found on hematopoietic stem cells, pro-B cells, normal plasma cells or other normal tissues (Tedder et al. J. Immunol. 135(2):973-979 (1985)). CD20 is thought to regulate an early step(s) in the activation process for cell cycle initiation and differentiation (Tedder et al., supra) and possibly functions as a calcium ion channel (Tedder et al. J. Cell. Biochem. 14D:195 (1990)).

Given the expression of CD20 in B cell lymphomas, this antigen has been a useful therapeutic target to treat such lymphomas. There are more than 300,000 people in the United States with B-cell NHL and more than 56,000 new cases are diagnosed each year. For example, the rituximab (RITUXAN®) antibody which is a genetically engineered chimeric murine/human monoclonal antibody directed against human CD20 antigen (commercially available from Genentech, Inc., South San Francisco, Calif., U.S.) is used for the treatment of patients with relapsed or refractory low-grade or follicular, CD20 positive, B cell non-Hodgkin's lymphoma. Rituximab is the antibody referred to as “C2B8” in U.S. Pat. No. 5,736,137 issued Apr. 7, 1998 (Anderson et al.) and in U.S. Pat. No. 5,776,456. In vitro mechanism of action studies have demonstrated that RITUXAN® binds human complement and lyses lymphoid B cell lines through complement-dependent cytotoxicity (CDC) (Reff et al. Blood 83(2):435-445 (1994)). Additionally, it has significant activity in assays for antibody-dependent cellular cytotoxicity (ADCC). In vivo preclinical studies have shown that RITUXAN® depletes B cells from the peripheral blood, lymph nodes, and bone marrow of cynomolgus monkeys, presumably through complement and cell-mediated processes (Reff et al. Blood 83(2):435-445 (1994)). Other anti-CD20 antibodies indicated for the treatment of NHL include the murine antibody Zevalin™ which is linked to the radioisotope, Yttrium-90 (IDEC Pharmaceuticals, San Diego, Calif.), Bexxar™ which is a another fully murine antibody conjugated to I-131 (Corixa, Wash.).

CD20 is also a useful target antigen for treating autoimmune diseases. Rituximab has also been studied in a variety of non-malignant autoimmune disorders, in which B cells and autoantibodies appear to play a role in disease pathophysiology. Edwards et al., Biochem Soc. Trans. 30:824-828 (2002). Rituximab has been reported to potentially relieve signs and symptoms of, for example, rheumatoid arthritis (RA) (Leandro et al., Ann. Rheum. Dis. 61:883-888 (2002); Edwards et al., Arthritis Rheum., 46 (Suppl. 9): S46 (2002); Stahl et al., Ann. Rheum. Dis., 62 (Suppl. 1): OP004 (2003); Emery et al., Arthritis Rheum. 48(9): S439 (2003)), lupus (Eisenberg, Arthritis. Res. Ther. 5:157-159 (2003); Leandro et al. Arthritis Rheum. 46: 2673-2677 (2002); Gorman et al., Lupus, 13: 312-316 (2004)), immune thrombocytopenic purpura (D'Arena et al., Leuk. Lymphoma 44:561-562 (2003); Stasi et al., Blood, 98: 952-957 (2001); Saleh et al., Semin. Oncol., 27 (Supp 12):99-103 (2000); Zaia et al., Haematolgica, 87: 189-195 (2002); Ratanatharathorn et al., Ann. Int. Med., 133: 275-279 (2000)), pure red cell aplasia (Auner et al., Br. J. Haematol., 116: 725-728 (2002)); autoimmune anemia (Zaja et al., Haematologica 87:189-195 (2002) (erratum appears in Haematologica 87:336 (2002)), cold agglutinin disease (Layios et al., Leukemia, 15: 187-8 (2001); Berentsen et al., Blood, 103: 2925-2928 (2004); Berentsen et al., Br. J. Haematol., 115: 79-83 (2001); Bauduer, Br. J. Haematol., 112: 1083-1090 (2001); Damiani et al., Br. J. Haematol., 114: 229-234 (2001)), type B syndrome of severe insulin resistance (Coll et al., N. Engl. J. Med., 350: 310-311 (2004), mixed cryoglobulinemia (DeVita et al., Arthritis Rheum. 46 Suppl. 9:S206/S469 (2002)), myasthenia gravis (Zaja et al., Neurology, 55: 1062-63 (2000); Wylam et al., J. Pediatr., 143: 674-677 (2003)), Wegener's granulomatosis (Specks et al., Arthritis & Rheumatism 44: 2836-2840 (2001)), refractory pemphigus vulgaris (Dupuy et al., Arch Dermatol., 140:91-96 (2004)), dermatomyositis (Levine, Arthritis Rheum., 46 (Suppl. 9):S1299 (2002)), Sjogren's syndrome (Somer et al., Arthritis & Rheumatism, 49: 394-398 (2003)), active type-II mixed cryoglobulinemia (Zaja et al., Blood, 101: 3827-3834 (2003)), pemphigus vulgaris (Dupay et al., Arch. Dermatol., 140: 91-95 (2004)), autoimmune neuropathy (Pestronk et al., J. Neurol. Neurosurg. Psychiatry 74:485-489 (2003)), paraneoplastic opsoclonus-myoclonus syndrome (Pranzatelli et al. Neurology 60(Suppl. 1) PO5.128:A395 (2003)), and relapsing-remitting multiple sclerosis (RRMS). Cross et al. (abstract) “Preliminary results from a phase II trial of Rituximab in MS” Eighth Annual Meeting of the Americas Committees for Research and Treatment in Multiple Sclerosis, 20-21 (2003).

A Phase II clinical trial has been conducted in patients with rheumatoid arthritis (RA), providing 48-week follow-up data on safety and efficacy of Rituximab. Emery et al. Arthritis Rheum 48(9):S439 (2003); Szczepanski et al. Arthritis Rheum 48(9):S121 (2003). Patients were evenly randomized to four treatment arms: methotrexate, rituximab alone, rituximab plus methotrexate, and rituximab plus cyclophosphamide (CTX). The treatment regimen of rituximab was one gram administered intravenously on days 1 and 15.

Publications concerning therapy with rituximab include: Perotta and Abuel, “Response of chronic relapsing ITP of 10 years duration to rituximab” Abstract # 3360 Blood 10(1)(part 1-2): p. 88B (1998); Perotta et al., “Rituxan in the treatment of chronic idiopathic thrombocytopenic purpura (ITP)”, Blood, 94: 49 (abstract) (1999); Matthews, R., “Medical Heretics” New Scientist (7 Apr. 2001); Leandro et al., “Clinical outcome in 22 patients with rheumatoid arthritis treated with B lymphocyte depletion” Ann Rheum Dis, supra; Leandro et al., “Lymphocyte depletion in rheumatoid arthritis: early evidence for safety, efficacy and dose response” Arthritis and Rheumatism 44(9): S370 (2001); Leandro et al., “An open study of B lymphocyte depletion in systemic lupus erythematosus”, Arthritis and Rheumatism, 46:2673-2677 (2002), wherein during a 2-week period, each patient received two 500-mg infusions of rituximab, two 750-mg infusions of cyclophosphamide, and high-dose oral corticosteroids, and wherein two of the patients treated relapsed at 7 and 8 months, respectively, and have been retreated, although with different protocols; “Successful long-term treatment of systemic lupus erythematosus with rituximab maintenance therapy” Weide et al., Lupus, 12: 779-782 (2003), wherein a patient was treated with rituximab (375 mg/m²×4, repeated at weekly intervals) and further rituximab applications were delivered every 5-6 months and then maintenance therapy was received with rituximab 375 mg/m² every three months, and a second patient with refractory SLE was treated successfully with rituximab and is receiving maintenance therapy every three months, with both patients responding well to rituximab therapy; Edwards and Cambridge, “Sustained improvement in rheumatoid arthritis following a protocol designed to deplete B lymphocytes” Rheumatology 40:205-211 (2001); Cambridge et al., “B lymphocyte depletion in patients with rheumatoid arthritis: serial studies of immunological parameters” Arthritis Rheum., 46 (Suppl. 9): S1350 (2002); Edwards et al., “B-lymphocyte depletion therapy in rheumatoid arthritis and other autoimmune disorders” supra; Edwards et al., “Efficacy and safety of rituximab, a B-cell targeted chimeric monoclonal antibody: A randomized, placebo controlled trial in patients with rheumatoid arthritis. Arthritis and Rheumatism 46(9): S197 (2002); Levine and Pestronk, “IgM antibody-related polyneuropathies: B-cell depletion chemotherapy using rituximab” Neurology 52: 1701-1704 (1999); DeVita et al., “Efficacy of selective B cell blockade in the treatment of rheumatoid arthritis” Arthritis & Rheum 46:2029-2033 (2002); Hidashida et al. “Treatment of DMARD-refractory rheumatoid arthritis with rituximab.” Presented at the Annual Scientific Meeting of the American College of Rheumatology; Oct. 24-29; New Orleans, La. 2002; Tuscano, J. “Successful treatment of infliximab-refractory rheumatoid arthritis with rituximab” Presented at the Annual Scientific Meeting of the American College of Rheumatology; Oct. 24-29; New Orleans, La. 2002; “Pathogenic roles of B cells in human autoimmunity; insights from the clinic” Martin and Chan, Immunity 20:517-527 (2004); Silverman and Weisman, “Rituximab Therapy and Autoimmune Disorders, Prospects for Anti-B Cell Therapy”, Arthritis and Rheumatism, 48: 1484-1492 (2003); Kazkaz and Isenberg, “Anti B cell therapy (rituximab) in the treatment of autoimmune diseases”, Current opinion in pharmacology, 4: 398-402 (2004); Virgolini and Vanda, “Rituximab in autoimmune diseases”, Biomedicine & pharmacotherapy, 58: 299-309(2004); Klemmer et al., “Treatment of antibody mediated autoimmune disorders with a AntiCD20 monoclonal antibody Rituximab”, Arthritis And Rheumatism, 48: (9) 9,S (SEP), page: S624-S624(2003); Kneitz et al., “Effective B cell depletion with rituximab in the treatment of autoimmune diseases”, Immunobiology, 206: 519-527 (2002); Arzoo et al., “Treatment of refractory antibody mediated autoimmune disorders with an anti-CD20 monoclonal antibody (rituximab)” Annals of the Rheumatic Diseases, 61 (10), p922-4 (2002) Comment in Ann Rheum Dis. 61: 863-866 (2002); “Future Strategies in Immunotherapy” by Lake and Dionne, in Burger's Medicinal Chemistry and Drug Discovery (2003 by John Wiley & Sons, Inc.) Article Online Posting Date: Jan. 15, 2003 (Chapter 2“ Antibody-Directed Immunotherapy”); Liang and Tedder, Wiley Encyclopedia of Molecular Medicine, Section: CD20 as an Immunotherapy Target, article online posting date: 15 Jan. 2002 entitled “CD20”; Appendix 4A entitled “Monoclonal Antibodies to Human Cell Surface Antigens” by Stockinger et al., eds: Coligan et al., in Current Protocols in Immunology (2003 John Wiley & Sons, Inc) Online Posting Date: May, 2003; Print Publication Date: February, 2003; Penichet and Morrison, “CD Antibodies/molecules: Definition; Antibody Engineering” in Wiley Encyclopedia of Molecular Medicine Section: Chimeric, Humanized and Human Antibodies; posted online 15 Jan. 2002; Specks et al. “Response of Wegener's granulomatosis to anti-CD20 chimeric monoclonal antibody therapy” Arthritis & Rheumatism 44:2836-2840 (2001); online abstract submission and invitation Koegh et al., “Rituximab for Remission Induction in Severe ANCA-Associated Vasculitis: Report of a Prospective Open-Label Pilot Trial in 10 Patients”, American College of Rheumatology, Session Number: 28-100, Session Title: Vasculitis, Session Type: ACR Concurrent Session, Primary Category: 28 Vasculitis, Session Oct. 18, 2004 (http://www.abstractsonline.com/viewer/SearchResults.asp); Eriksson, “Short-term outcome and safety in 5 patients with ANCA-positive vasculitis treated with rituximab”, Kidney and Blood Pressure Research, 26: 294 (2003); Jayne et al., “B-cell depletion with rituximab for refractory vasculitis” Kidney and Blood Pressure Research, 26: 294 (2003); Jayne, poster 88 (11^{th} International Vasculitis and ANCA workshop), 2003 American Society of Nephrology; Stone and Specks, “Rituximab Therapy for the Induction of Remission and Tolerance in ANCA-associated Vasculitis”, in the Clinical Trial Research Summary of the 2002-2003 Immune Tolerance Network, http://www.immunetolerance.org/research/autoimmune/trials/stone.html. See also Leandro et al., “B cell repopulation occurs mainly from naïve B cells in patient with rheumatoid arthritis and systemic lupus erythematosus” Arthritis Rheum., 48 (Suppl 9): S1160 (2003).

A major limitation in the use of murine antibodies in human therapy is the human anti-mouse antibody (HAMA) response (see, e.g., Miller, R. A. et al. “Monoclonal antibody therapeutic trials in seven patients with T-cell lymphoma” Blood, 62:988-995, 1983; and Schroff, R. W., et al. “Human anti-murine immunoglobulin response in patients receiving monoclonal antibody therapy” Cancer Res., 45:879-885, 1985). Even chimeric molecules, where the variable (V) domains of rodent antibodies are fused to human constant (C) regions, are still capable of eliciting a significant immune response (HACA, human anti-chimeric antibody) (Neuberger et al. Nature (Lond.), 314:268-270, 1985). A powerful approach to overcome these limitations in the clinical use of monoclonal antibodies is “humanization” of the murine antibody or antibody from a non-human species (Jones et al. Nature (Lond), 321:522-525, 1986; Riechman et al., Nature (Lond), 332:323-327, 1988). Such antibodies are expected to create minimal or no antigenicity when administered to patients, especially for chronic treatment. Humanized anti-CD20 antibodies have been described in WO 03/068821 A2 (Hansen et al), WO2004103404 (Watkins), and WO 04/056312 (Adams). Human anti-CD20 antibodies have been described in WO 2004/035607 (Teeling et al.).

It would be beneficial to provide therapeutic CD20 binding antibodies that not only have minimal antigenicity but have improved biological properties and clinical efficacy. The present invention satisfies this and other needs. The present invention provides anti-CD20 antibodies that overcome the limitations of current therapeutic compositions as well as offer additional advantages that will be apparent from the detailed description below.

Patents and patent publications concerning CD20 antibodies include U.S. Pat. Nos. 5,776,456, 5,736,137, 5,843,439, 6,399,061, and 6,682,734, as well as US patent application nos. US 2002/0197255A1, US 2003/0021781A1, US 2003/0082172 A1, US 2003/0095963 A1, US 2003/0147885 A1 (Anderson et al.); U.S. Pat. No. 6,455,043B1 and WO00/09160 (Grillo-Lopez, A.); WO00/27428 (Grillo-Lopez and White); WO00/27433 (Grillo-Lopez and Leonard); WO00/44788 (Braslawsky et al.); WO01/10462 (Rastetter, W.); WO01/10461 (Rastetter and White); WO01/10460 (White and Grillo-Lopez); US2001/0018041 A1, US2003/0180292A1, WO01/34194 (Hanna and Hariharan); US appln no. US2002/0006404 and WO02/04021 (Hanna and Hariharan); US appln no. US2002/0012665 A1 and WO01/74388 (Hanna, N.); US appln no. US 2002/0058029 A1 (Hanna, N.); US appln no. US 2003/0103971 A1 (Hariharan and Hanna); US appln no. US2002/0009444A1, and WO01/80884 (Grillo-Lopez, A.); WO01/97858 (White, C.); US appln no. US2002/0128488A1 and WO02/34790 (Reff, M.); WO02/060955 (Braslawsky et al.); WO2/096948 (Braslawsky et al.); WO02/079255 (Reff and Davies); U.S. Pat. No. 6,171,586B1, and WO98/56418 (Lam et al.); WO98/58964 (Raju, S.); WO99/22764 (Raju, S.); WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al.); WO00/42072 (Presta, L.); WO00/67796 (Curd et al.); WO01/03734 (Grillo-Lopez et al.); US appln no. US 2002/0004587A1 and WO01/77342 (Miller and Presta); US appln no. US2002/0197256 (Grewal, I.); US Appln no. US 2003/0157108 A1 (Presta, L.); U.S. Pat. Nos. 6,565,827B1, 6,090,365B1, 6,287,537B1, 6,015,542, 5,843,398, and 5,595,721, (Kaminski et al.); U.S. Pat. Nos. 5,500,362, 5,677,180, 5,721,108, 6,120,767, 6,652,852B1 (Robinson et al.); U.S. Pat. No. 6,410,391B1 (Raubitschek et al.); U.S. Pat. No. 6,224,866B1 and WO00/20864 (Barbera-Guillem, E.); WO01/13945 (Barbera-Guillem, E.); WO00/67795 (Goldenberg); US Appl No. US 2003/0133930 A1 and WO00/74718 (Goldenberg and Hansen); WO00/76542 (Golay et al.); WO01/72333 (Wolin and Rosenblatt); U.S. Pat. No. 6,368,596B1 (Ghetie et al.); U.S. Pat. No. 6,306,393 and US Appln no. US2002/0041847 A1, (Goldenberg, D.); US Appln no. US2003/0026801A1 (Weiner and Hartmann); WO02/102312 (Engleman, E.); US Patent Application No. 2003/0068664 (Albitar et al.); WO03/002607 (Leung, S.); WO 03/049694, US2002/0009427A1, and US 2003/0185796 A1 (Wolin et al.); WO03/061694 (Sing and Siegall); US 2003/0219818 A1 (Bohen et al.); US 2003/0219433 A1 and WO 03/068821 (Hansen et al.); US2002/0136719A1 (Shenoy et al.); WO2004/032828 (Wahl et al.); WO2004/035607 (Teeling et al.); US2004/0093621 (Shitara et al.). See also U.S. Pat. No. 5,849,898 and EP appln no. 330,191 (Seed et al.); U.S. Pat. No. 4,861,579 and EP332,865A2 (Meyer and Weiss); WO95/03770 (Bhat et al.), US 2001/0056066 (Bugelski et al.); WO 2004/035607 (Teeling et al.); WO 2004/056312 (Lowman et al.); US 2004/0093621 (Shitara et al.); and WO 2004/103404 (Watkins et al.). Publications concerning CD20 antibody include: Teeling, J. et al “Characterization of new human CD20 monoclonal antibodies with potent cytolytic activity against non-Hodgkin's lymphomas” Blood, June 2004; 10.1182.

### SUMMARY OF THE INVENTION

The invention provides humanized 2H7 variant antibodies listed in Tables 13 and 14 that bind human CD20 and deplete primate B cells in vivo. In specific embodiments, the hu2H7 antibodies are version 472, 473, 511, 523 and 516. In preferred embodiments of the compositions, methods, article of manufacture, formulation, of the invention, the humanized 2H7 antibody is hu2H7.v511 or hu2H7.v114.

The invention provides a humanized 2H7 antibody that binds human CD20, or an antigen-binding fragment thereof, wherein the antibody is effective to deplete primate B cells in vivo, the antibody comprising the L chain Variable region (V_{L}) sequence of SEQ ID NO. 25 and the H chain Variable region (V_{H}) sequence of SEQ ID NO. 8, but with an amino acid substitution of D56A in VH-CDR2, and N100 in VH-CDR3 is substituted with Y or W. In one embodiment this antibody further comprises the substitution S100aR in VH-CDR3. In a further embodiment of the preceding antibodies, antibody further comprises at least one amino acid substitution in the Fc region that improves ADCC and/or CDC activity. To improve the ADCC and/or CDC effector functions, in one embodiment, the preceding antibodies will further comprising an IgG1 Fc comprising the amino acid substitutions S298A, E333A, K334A, and either K326A or K326W. In one embodiment the antibody of the preceding embodiments exhibits at least 20 fold greater antibody dependent cellular cytotoxicity (ADCC) than 2H7.v 16 and exhibits at least 25 fold greater complement cytotoxicity than 2H7.v16.

Alternatively, the antibody that comprises the substitution S100aR can have at least one amino acid alteration or substitution in the Fc that improves ADCC but decreases CDC activity. In that respect, the antibody can comprise at least the amino acid substitution K322A in the Fc and can further comprise the amino acid substitutions S298A, E333A, K334A.

In preferred embodiments, the antibody that has improved ADCC and CDC activity also binds human CD20 with at least 3 fold, preferably at least 6 fold increased affinity relative to antibody 2H7.v16 which has L chain and H chain sequence of SEQ ID NO. 13 and 14, respectively.

The invention provides a humanized 2H7 antibody that binds human CD20 wherein the antibody consists of the light chain sequence of SEQ ID NO.26 and heavy chain sequence of SEQ ID NO. 34.

Also provided by the invention is an antibody of any of the preceding embodiments, conjugated to a cytotoxic agent. In one embodiment, the cytotoxic agent is a radioactive isotope or a toxin.

In one embodiment the antibodies of the invention are produced in CHO cells.

The invention further provides an isolated nucleic acid that encodes an antibody of the above embodiments, including an expression vector. Provided as well is a host cell comprising the nucleic acid produces the antibody. In one embodiment the host cell is one that produces the antibody 2H7.v511.

A method is provided for producing any of the above antibodies, comprising culturing the preceding host cell and recovering the antibody from the cell culture.

Also provided is a composition comprising the 2H7 antibody of the invention and a carrier. In one embodiment the carrier is a pharmaceutically acceptable carrier.

Another embodiment is a composition comprising hu2H7.v511 wherein about 80-100% of the antibodies in the composition lacks fucose.

Yet another aspect is an article of manufacture comprising a container and a composition contained therein, wherein the composition comprises an antibody of any of the preceding embodiments. Depending on the specific treatment desired of the composition, the article of manufacture can further comprising a package insert indicating that the composition is used to treat non-Hodgkin's lymphoma or composition is used to treat rheumatoid arthritis.

Yet another aspect of the invention is a method of depleting B cells in vivo using a humanized 2H7 antibody of the preceding embodiments.

The invention also provides a method of treating a CD20 positive cancer, comprising administering to a patient having the cancer, a therapeutically effective amount of the humanized 2H7 antibody of the preceding embodiments. The CD20 positive cancer is preferably a B cell lymphoma or leukemia. In specific embodiments, the humanized 2H7 antibodies that bind hCD20 and functional fragments thereof are used to treat non-Hodgkin's lymphoma (NHL), lymphocyte predominant Hodgkin's disease (LPHD), small lymphocytic lymphoma (SLL), chronic lymphocytic leukemia (CLL). In specific embodiments, humanized CD20 binding antibodies or functional fragments thereof, in particular v511 and v114 are used to treat indolent NHL including relapsed indolent NHL and rituximab-refractory indolent NHL.

For treating these cancers, in one embodiment, the antibody is administered via intravenous infusion. The dosage administered via infusion is in the range of about 12.5 mg/m² to 800 mg/m² per dose, generally one dose per week for a total of one, two, three or four doses.

The invention also provides a method of alleviating an autoimmune disease, comprising administering to a patient suffering from the autoimmune disease, a therapeutically effective amount of a humanized 2H7 antibodies of any one of the preceding embodiments. In preferred embodiments the antibody is administered intravenously or subcutaneously. The antibody is administered intravenously at a dosage in the range of 10 mg to 500 mg per dose and in a specific embodiment, the dosage is 100 mg/dose.

In specific embodiments, the autoimmune disease is selected from the group consisting of rheumatoid arthritis and juvenile rheumatoid arthritis, systemic lupus erythematosus (SLE) including lupus nephritis, Wegener's disease, inflammatory bowel disease, ulcerative colitis, idiopathic thrombocytopenic purpura (ITP), thrombotic thrombocytopenic purpura (TTP), autoimmune thrombocytopenia, multiple sclerosis, psoriasis, IgA nephropathy, IgM polyneuropathies, myasthenia gravis, ANCA associated vasculitis, diabetes mellitus, Reynaud's syndrome, Sjogren's syndrome, Neuromyelitis Optica (NMO) and glomerulonephritis.

The hu2H7.v511 and hu2H7.v114 antibodies are useful in a method to treat or alleviate an autoimmune disease or a B cell neoplasm in conjuction with a second therapeutic agent, either concurrently, sequentially or in alternating regimen. In one embodiment, the second therapeutic agent is a BAFF antagonist In certain embodiments, the BAFF antagonist is an antibody that binds human BR3 or a BR3-Fc fusion protein.

In a preferred embodiment of the method of treating the aforementioned diseases, the subject or patient suffering from the disease is a primate, preferably a human.

Also provided is a liquid formulation comprising humanized 2H7.v511 antibody at about 20 mg/ml, 20 mM sodium acetate, 4% trehalose dihydrate, 0.02% polysorbate 20, pH 5.5, for intravenous administration. A liquid formulation comprising humanized 2H7.v114 antibody at about 20 mg/ml, in 20 mM sodium acetate, 240 mM (8%) trehalose dihydrate, pH 5.3, 0.02% Polysorbate 20 is also provided. Another liquid formulation that can be used for 2H7v16 is about 30 mg/ml antibody in 20 mM sodium acetate, pH 5.3, 4% trehalose dehydrate, 0.02% polysorbate 20 (Tween 20™).

### BRIEF DESCRIPTION OF THE FIGURES

FIG. 1A shows a sequence alignment comparing the amino acid sequences of the light chain variable domain (V_{L}) of each of murine 2H7 (SEQ ID NO. 1), humanized 2H7. v16 variant (SEQ ID NO. 2), and human kappa light chain subgroup I (SEQ ID NO. 3). The CDRs of V_{L} of 2H7 and hu2H7.v16 are as follows: CDR1 (SEQ ID NO.4), CDR2 (SEQ ID NO.5), and CDR3 (SEQ ID NO.6).

FIG. 1B shows a sequence alignment which compares the V_{H} sequences of murine 2H7 (SEQ ID NO. 7), humanized 2H7.v16 variant (SEQ ID NO. 8), and the human consensus sequence of heavy chain subgroup III (SEQ ID NO. 9). The CDRs of V_{H} of 2H7 and hu2H7.v16 are as follow: CDR1 (SEQ ID NO.10), CDR2 (SEQ ID NO.11), and CDR3 (SEQ ID NO.12).

In FIG. 1A and FIG. 1B, the CDR1, CDR2 and CDR3 in each chain are enclosed within brackets, flanked by the framework regions, FR1-FR4, as indicated. 2H7 refers to the murine 2H7 antibody. The asterisks in between two rows of sequences indicate the positions that are different between the two sequences. Residue numbering is according to Kabat et al., Sequences of Immunological Interest. 5th Ed. Public Health Service, National Institutes of Health, Bethesda, Md. (1991), with insertions shown as a, b, c, d, and e.

FIG. 2A shows the protein sequence alignment of the L chains of humanized 2H7.v 16 versus v511.

FIG. 2B shows the protein sequence alignment of the H chains of humanized 2H7.v 16 versus v511.

FIG. 3 shows relative binding of 2H7 variants in a solubilized CD20 ELISA. Antibodies 2H7.v16 (circles), 2H7.v511 (squares), and 2H7.v588 (triangles) were compared (see Example 12).

FIG. 4 shows ADCC activity using WIL2-S cells and purified human NK cells. Antibodies 2H7.v16 (circles), 2H7.v511 (squares), and 2H7.v588 (triangles) were compared for their ability to mediate ADCC in vitro (see Example 13).

FIG. 5 shows CDC activity using WIL2-S cells and human complement. Antibodies 2H7.v16 (circles), 2H7.v511 (squares), and 2H7.v588 (triangles) were compared for their ability to mediate CDC in vitro (see Example 14).

FIG. 6 shows B-cell depletion in the blood (left) and peritoneal cavity (right) by 2H7 variants v16 and v511 in vivo at day 2 after antibody injection (see Example 15).

FIG. 7 shows B-cell depletion in the spleen by 2H7 variants v16 and v511 two days after antibody injection: splenic B-cells(left), marginal zone B-cells (middle), and follicular B-cells (right) (see Example 15).

FIG. 8 compares the in vivo B cell depletion by 2H7.v16 and .v511 in hCD20 Tg+/+/mCD16−/−/hCD16 Tg+/− transgenic mice which express human CD20 and human CD16 (see Example 15).

FIG. 9 shows the vector for expression of 2H7.v16 in CHO cells.

FIG. 10 shows a comparison of 2H7.v511 to other 2H7 variants v16, v31, v114, v138, and v488 in complement (CDC) activity as assayed on WIL2-S cells (see Example 14).

FIG. 11 compares the ADCC activity of antibody variants 2H7.v511, v16, v31, v114, v138, and v488 using WIL2-S cells and purified human NK cells, as described in Example 13 and FIG. 4.

FIG. 12 outlines the in vivo B cell depletion analysis of 2H7.v511 and rituximab (Rituxan®) in hCD20 Tg+/+/mCD16−/−/hCD16 Tg+/− transgenic mice which express human CD20 and human CD16; the activities of the two antibodies are summarized in the table (see Example 15).

FIG. 13 shows B-cell depletion in the blood (left) and peritoneal cavity (right) by 2H7.v511 and rituximab in vivo at day 2 after injection (see Example 15).

FIG. 14 shows B-cell depletion in the spleen by 2H7.v511 and rituximab at day 2 after antibody injection: splenic B-cells(left), marginal zone B-cells (middle), and follicular B-cells (right) (see Example 15).

FIGS. 15A, 15B, and 15C show a comparison of CDC activity in vitro of PBMCs purified from 3 CLL patients where the PBMC were treated with 2H7.v114 or Rituxan™, and human serum complement.

FIGS. 16A, 16B, and 16C show a comparison of CDC activity in vitro of PMBCs from 3 CLL patients (the same patients as in FIG. 15), where the PBMC were treated with 2H7.v511 or Rituxan™, and human serum complement.

### DETAILED DESCRIPTION OF THE PREFERRED EMBODIMENTS

The “CD20” antigen is a non-glycosylated, transmembrane phosphoprotein with a molecular weight of approximately 35 kD that is found on the surface of greater than 90% of B cells from peripheral blood or lymphoid organs. CD20 is expressed during early pre-B cell development and remains until plasma cell differentiation; it is not found on human stem cells, lymphoid progenitor cells or normal plasma cells. CD20 is present on both normal B cells as well as malignant B cells. Other names for CD20 in the literature include “B-lymphocyte-restricted differentiation antigen” and “Bp35”. The CD20 antigen is described in, for example, Clark and Ledbetter, Adv. Can. Res. 52:81-149 (1989) and Valentine et al. J. Biol. Chem. 264(19):11282-11287 (1989).

The term “antibody” is used in the broadest sense and specifically covers monoclonal antibodies (including full length monoclonal antibodies), multispecific antibodies (e.g., bispecific antibodies), and antibody fragments so long as they exhibit the desired biological activity or function.

The biological activity of the humanized CD20 binding antibodies of the invention will include at least binding of the antibody to human CD20, more preferably binding to human and other primate CD20 (including cynomolgus monkey, rhesus monkey, chimpanzees). The antibodies would bind CD20 with a K_{d} value of no higher than 1×10⁻⁸, preferably a K_{d} value no higher than about 1×10⁹, and be able to kill or deplete B cells in vivo, preferably by at least 20% when compared to the appropriate negative control which is not treated with such an antibody. B cell depletion can be a result of one or more of ADCC, CDC, apoptosis, or other mechanism. In some embodiments of disease treatment herein, specific effector functions or mechanisms may be desired over others and certain variants of the humanized 2H7 are preferred to achieve those biological functions, such as ADCC.

“Antibody fragments” comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981)), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol. 340(5):1073-1093 (2004); Fellouse, Proc. Nat. Acad. Sci. USA 101(34):12467-12472 (2004); and Lee et al. J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806; 5,569,825; 5,591,669 (all of GenPharm); 5,545,807; WO 1997/17852; U.S. Pat. Nos. 5,545,807; 5,545,806; 5,569,825; 5,625,126; 5,633,425; and 5,661,016; Marks et al., Bio/Technology, 10: 779-783 (1992); Lonberg et al., Nature, 368: 856-859 (1994); Morrison, Nature, 368: 812-813 (1994); Fishwild et al., Nature Biotechnoloy, 14: 845-851 (1996); Neuberger, Nature Biotechnology, 14: 826 (1996); and Lonberg and Huszar, Intern. Rev. Immunol., 13: 65-93 (1995).

“Functional fragments” of the CD20 binding antibodies of the invention are those fragments that retain binding to CD20 with substantially the same affinity as the intact full length molecule from which they are derived and show biological activity including depleting B cells as measured by in vitro or in vivo assays such as those described herein.

The term “variable” refers to the fact that certain segments of the variable domains differ extensively in sequence among antibodies. The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

As referred to herein, the “consensus sequence” or consensus V domain sequence is an artificial sequence derived from a comparison of the amino acid sequences of known human immunoglobulin variable region sequences. Based on these comparisons, recombinant nucleic acid sequences encoding the V domain amino acids that are a consensus of the sequences derived from the human κ and the human H chain subgroup III V domains were prepared. The consensus V sequence does not have any known antibody binding specificity or affinity.

“Chimeric” antibodies (immunoglobulins) have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Humanized antibody as used herein is a subset of chimeric antibodies.

“Humanized” forms of non-human (e.g., murine) antibodies are chimeric antibodies which contain minimal sequence derived from non-human immunoglobulin. For the most part, humanized antibodies are human immunoglobulins (recipient or acceptor antibody) in which hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence although the FR regions may include one or more amino acid substitutions that improve binding affinity. The number of these amino acid substitutions in the FR are typically no more than 6 in the H chain, and in the L chain, no more than 3. The humanized antibody optionally also will comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992).

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g. B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or Presta U.S. Pat. No. 6,737,056 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998). Where the antibody is a CD20 binding antibody, ADCC activity can be tested in transgenic mice expressing human CD20 plus CD16 (hCD20+/hCD16+Tg mice) as described below.

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g. from blood.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulates homeostasis of immunoglobulins.

WO00/42072 (Presta) describes antibody variants with improved or diminished binding to FcRs. The content of that patent publication is specifically incorporated herein by reference. See, also, Shields et al. J. Biol. Chem. 9(2): 6591-6604 (2001).

For binding affinity to FcRn, in one embodiment, the EC50 or apparent Kd (at pH 6.0) of the antibody is <=100 nM, more preferably <=10 nM. For increased binding affinity to FcγRIII (F158; i.e. low-affinity isotype), in one embodiment the EC50 or apparent Kd<=10 nM, and for FcgRIII (VI 58; high-affinity) the EC50 or apparent Kd<=3 nM. Methods of measuring binding to FcRn are known (see, e.g., Ghetie 1997, Hinton 2004) as well as described below. Binding to human FcRn in vivo and serum half life of human FcRn high affinity binding polypeptides can be assayed, e.g., in transgenic mice or transfected human cell lines expressing human FcRn, or in primates administered with the Fc variant polypeptides. In certain embodiments, the humanized 2H7 antibody of the invention further comprises amino acid alterations in the IgG Fc and exhibits increased binding affinity for human FcRn over an antibody having wild-type IgG Fc, by at least 60 fold, at least 70 fold, at least 80 fold, more preferably at least 100 fold, preferably at least 125 fold, even more preferably at least 150 fold to about 170 fold.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g. as described in Gazzano-Santoro et al., J. Immunol. Methods 202:163 (1996), may be performed.

Polypeptide variants with altered Fc region amino acid sequences and increased or decreased C1q binding capability are described in U.S. Pat. No. 6,194,551 B1 and WO99/51642. The contents of those patent publications are specifically incorporated herein by reference. See, also, Idusogie et al. J. Immunol. 164: 4178-4184 (2000).

Throughout the present specification and claims, unless otherwise indicated, the numbering of the residues in the constant domains of an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody. The residues in the V region are numbered according to Kabat numbering unless sequential or other numbering system is specifically indicated.

CD20 antibodies include: “C2B8,” which is now called “rituximab” (“RITUXAN®”) (U.S. Pat. No. 5,736,137); the yttrium-[90]-labelled 2B8 murine antibody designated “Y2B8” or “Ibritumomab Tiuxetan” (ZEVALIN®) commercially available from IDEC Pharmaceuticals, Inc. (U.S. Pat. No. 5,736,137; 2B8 deposited with ATCC under accession no. HB11388 on Jun. 22, 1993); murine IgG2a “B1,” also called “Tositumomab,” optionally labelled with ¹³¹I to generate the “131I-B1” or “iodine I131 tositumomab” antibody (BEXXAR™, GlaxoSmithKline, see, also, U.S. Pat. No. 5,595,721); murine monoclonal antibody “IF5” (Press et al. Blood 69(2):584-591 (1987) and variants thereof including “framework patched” or humanized IF5 (WO 2003/002607, Leung, S.; ATCC deposit HB-96450); murine 2H7 and chimeric 2H7 antibody (U.S. Pat. No. 5,677,180); a humanized 2H7 (WO 2004/056312 (Lowman et al.) and as set forth below); HuMAX-CD20™ a fully human antibody (Genmab, Denmark; see, for example, Glennie and van de Winkel, Drug Discovery Today 8: 503-510 (2003) and Cragg et al., Blood 101: 1045-1052 (2003)); the human monoclonal antibodies set forth in WO 2004/035607 (Teeling et al.); the antibodies having complex N-glycoside-linked sugar chains bound to the Fc region described in US 2004/0093621 (Shitara et al.); CD20 binding molecules such as the AME series of antibodies, e.g., AME-133™ antibodies as set forth in WO 2004/103404 (Watkins et al., Applied Molecular Evolution); A20 antibody or variants thereof such as chimeric or humanized A20 antibody (cA20, IMMU-106 a.k.a. hA20, respectively (US 2003/0219433, US 2005/0025764; Immunomedics); and monoclonal antibodies L27, G28-2, 93-1B3, B-C1 or NU-B2 available from the International Leukocyte Typing Workshop (Valentine et al., In: Leukocyte Typing III (McMichael, Ed., p. 440, Oxford University Press (1987)). The preferred CD20 antibodies herein are humanized, chimeric, or human CD20 antibodies, more preferably, a humanized 2H7 antibody, rituximab, chimeric or humanized A20 antibody (Immunomedics), and HuMAX-CD20 ™ human CD20 antibody (Genmab).

As used herein, “B cell depletion” refers to a reduction in B cell levels in an animal or human after drug or antibody treatment, as compared to the level before treatment. B cell levels are measurable using well known assays such as by getting a complete blood count, by FACS analysis staining for known B cell markers, and by methods such as described in the Experimental Examples. B cell depletion can be partial or complete. In one embodiment, the depletion of CD20 expressing B cells is at least 25%. In a patient receiving a B cell depleting drug, B cells are generally depleted for the duration of time when the drug is circulating in the patient's body and the time for recovery of B cells.

“Tumor necrosis factor alpha (TNFα)” refers to a human TNFα molecule comprising the amino acid sequence as described in Pennica et al., Nature, 312:721 (1984) or Aggarwal et al., JBC, 260:2345 (1985). A “TNFα inhibitor” herein is an agent that inhibits, to some extent, a biological function of TNFα, generally through binding to TNFα and neutralizing its activity. Examples of TNF inhibitors specifically contemplated herein are Etanercept (ENBREL®), Infliximab (REMICADE®) and Adalimumab (HUMIRA™).

The term “inadequate response to a TNFα-inhibitor” refers to an inadequate response to previous or current treatment with a TNFα-inhibitor because of toxicity and/or inadequate efficacy. The inadequate response can be assessed by a clinician skilled in treating the disease in question. A mammal who experiences “inadequate efficacy” continues to have active disease following previous or current treatment with a TNFα-inhibitor. For instance, the patient may have active disease activity after 3 months of therapy with the TNFα-inhibitor.

The terms “BAFF,” “BAFF polypeptide,” “TALL-1” or “TALL-1 polypeptide,” “BLys”, “THANK” when used herein encompass “native sequence BAFF polypeptides” and “BAFF variants”. “BAFF” is a designation given to those polypeptides which are encoded by any one of the amino acid sequences shown below:

| Human BAFF sequence (SEQ ID NO: 46) | |
|---|---|
| 1 | mddstereqs rltsclkkre emklkecvsi lprkespsvr sskdgkllaa tlllallscc |
| 61 | ltvvsfyqva alqgdlaslr aelqghhaek lpagagapka gleeapavta glkifeppap |
| 121 | gegnssqnsr nkravqgpee tvtqdclqli adsetptiqk gsytfvpwll sfkrgsalee |
| 181 | kenkilvket gyffiygqvl ytdktyamgh liqrkkvhvf gdelslvtlf rciqnmpetl |
| 241 | pnnscysagi akleegdelq laiprenaqi sldgdvtffg alkll |

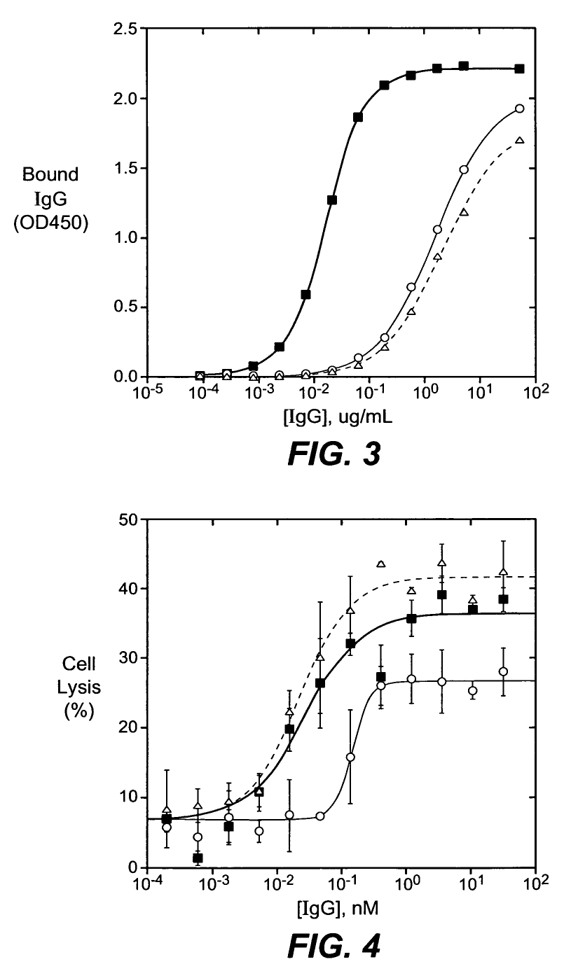

(see also sequence of FIG. 3 in US 2005/0095243A1)

and homologs and fragments and variants thereof, which have the biological activity of the native sequence BAFF. A biological activity of BAFF can be selected from the group consisting of promoting B cell survival, promoting B cell maturation and binding to BR3. The term “BAFF” includes those polypeptides described in Shu et al., J. Leukocyte Biol., 65:680 (1999); GenBank Accession No. AF136293; WO98/18921 published May 7, 1998; EP 869,180 published Oct. 7, 1998; WO98/27114 published Jun. 25, 1998; WO99/12964 published Mar. 18, 1999; WO99/33980 published Jul. 8, 1999; Moore et al., Science, 285:260-263 (1999); Schneider et al., J. Exp. Med., 189:1747-1756 (1999); Mukhopadhyay et al., J. Biol. Chem., 274:15978-15981 (1999).

The term “BAFF antagonist” as used herein is used in the broadest sense, and includes any molecule that (1) binds a native sequence BAFF polypeptide or binds a native sequence BR3 polypeptide to partially or fully block BR3 interaction with BAFF polypeptide, and (2) partially or fully blocks, inhibits, or neutralizes native sequence BAFF signaling. Native sequence BAFF polypeptide signaling promotes, among other things, B cell survival and B cell maturation. The inhibition, blockage or neutralization of BAFF signaling results in, among other things, a reduction in the number of B cells. A BAFF antagonist according to this invention will partially or fully block, inhibit, or neutralize one or more biological activities of a BAFF polypeptide, in vitro or in vivo. In one embodiment, a biologically active BAFF potentiates any one or combination of the following events in vitro or in vivo: an increased survival of B cells, an increased level of IgG and/or IgM, an increased numbers of plasma cells, and processing of NF-κb2/100 to p52 NF-κb in splenic B cells (e.g., Batten, M et al., (2000) J. Exp. Med. 192:1453-1465; Moore, et al., (1999) Science 285:260-263; Kayagaki, et al., (2002) 10:515-524).

The terms “BR3”, “BR3 polypeptide” or “BR3 receptor” when used herein encompass “native sequence BR3 polypeptides” and “BR3 variants” (which are further defined herein). “BR3” is a designation given to those polypeptides comprising any one of the following polynucleotide sequences and homologs thereof:

| (a) human BR3 sequence (SEQ ID NO: 47) | |
|---|---|
| 1 | MRRGPRSLRG RDAPAPTPCV PAECFDLLVR HCVACGLLRT PRPKPAGASS PAPRTALQPQ |
| 61 | ESVGAGAGEA ALPLPGLLFG APALLGLALV LALVLVGLVS WRRRQRRLRG ASSAEAPDGD |
| 121 | KDAPEPLDKV IILSPGISDA TAPAWPPPGE DPGTTPPGHS VPVPATELGS TELVTTKTAG |
| 181 | PEQQ |
| (b) alternative human BR3 sequence (SEQ ID NO: 48) | |
| 1 | MRRGPRSLRG RDAPAPTPCV PAECFDLLVR HCVACGLLRT PRPKPAGAAS SPAPRTALQP |
| 61 | QESVGAGAGE AALPLPGLLF GAPALLGLAL VLALVLVGLV SWRRRQRRLR GASSAEAPDG |
| 121 | DKDAPEPLDK VIILSPGISD ATAPAWPPPG EDPGTTPPGH SVPVPATELG STELVTTKTA |
| 181 | GPEQQ |

In some embodiments, a BAFF antagonist according to this invention includes anti-BAFF antibodies, BAFF-binding polypeptides (including immunoadhesins and peptides), and BAFF-binding small molecules. BAFF antagonists include the BAFF binding antibodies described in WO 02/02641 (e.g., antibodies comprising the amino acid sequence of any of SEQ ID NOs. 1-46, 321-329, 834-872, 1563-1595, 1881-1905 of Table 1). In a further embodiment, the immunoadhesin comprises a BAFF binding region of a BAFF receptor (e.g., an extracellular domain of BR3, BCMA or TACI). In a still further embodiment, the immunoadhesin is BR3-Fc. Other examples of BAFF binding Fc proteins can be found in WO 02/66516, WO 00/40716, WO 01/87979, WO 03/024991, WO 02/16412, WO 02/38766, WO 02/092620, WO 01/12812. Methods of making BAFF antagonists are described in US 2005/0095243A1, supra and US 2005/0163775).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Vector” includes shuttle and expression vectors. Typically, the plasmid construct will also include an origin of replication (e.g., the ColE1 origin of replication) and a selectable marker (e.g., ampicillin or tetracycline resistance), for replication and selection, respectively, of the plasmids in bacteria. An “expression vector” refers to a vector that contains the necessary control sequences or regulatory elements for expression of the antibodies including antibody fragment of the invention, in bacterial or eukaryotic cells. Suitable vectors are disclosed below.

The cell that produces a humanized CD20 binding antibody such as humanized 2H7 antibody of the invention will include the bacterial and eukaryotic host cells into which nucleic acid encoding the antibodies have been introduced. Suitable host cells are disclosed below.

The word “label” when used herein refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable.

Compositions and Methods of the Invention

The invention provides humanized 2H7 antibodies that are variants of 2H7.v16. In a specific embodiment, the variant is hu2H7.v511.

In a full length antibody, the humanized CD20 binding antibody of the invention will comprise a humanized V domain joined to a C domain of a human immunoglobulin. In a preferred embodiment, the H chain C region is from human IgG, preferably IgG1 or IgG3. The L chain C domain is preferably from human κ chain.

For the purposes herein, “humanized 2H7” refers to an intact antibody or antibody fragment comprising the variable light (V_{L}) sequence:

| | |
|---|---|
| DIQMTQSPSSLSASVGDRVTITCRASSSVSYMHWYQ | (SEQ ID NO: 2) |
| QKPGKAPKPLIYAPSNLASGVPSRFSGSGSGTDFTL | |
| TISSLQPEDFATYYCQQWSFNPPTFGQGTKVEIKR; | |
| and | |

variable heavy (V_{H}) sequence:

Where the humanized 2H7 antibody is an intact antibody, preferably it comprises the v16 light chain amino acid sequence:

| | |
|---|---|
| DIQMTQSPSSLSASVGDRVTITCRASSSVSYMHW | (SEQ ID NO: 13) |
| YQQKPGKAPKPLIYAPSNLASGVPSRFSGSGSGT | |
| DFTLTISSLQPEDFATYYCQQWSFNPPTFGQGTK | |
| VEIKRTVAAPSVFIFPPSDEQLKSGTASVVCLLN | |
| NFYPREAKVQWKVDNALQSGNSQESVTEQDSKDS | |
| TYSLSSTLTLSKADYEKHKVYACEVTHQGLSSPV | |
| TKSFNRGEC; | |
| and | |

heavy chain amino acid sequence:

| | |
|---|---|
| EVQLVESGGGLVQPGGSLRLSCAASGYTFTSYNM | (SEQ ID NO: 14) |
| HWVRQAPGKGLEWVGAIYPGNGDTSYNQKFKGRF | |
| TISVDKSKNTLYLQMNSLRAEDTAVYYCARVVYY | |
| SNSYWYFDVWGQGTLVTVSSASTKGPSVFPLAPS | |
| SKSTSGGTAALGCLVKDYFPEPVTVSWNSGALTS | |
| GVHTFPAVLQSSGLYSLSSVVTVPSSSLGTQTYI | |
| CNVNHKPSNTKVDKKVEPKSCDKTHTCPPCPAPE | |
| LLGGPSVFLFPPKPKDTLMISRTPEVTCVVVDVS | |
| HEDPEVKFNWYVDGVEVHNAKTKPREEQYNSTYR | |
| VVSVLTVLHQDWLNGKEYKCKVSNKALPAPIEKT | |
| ISKAKGQPREPQVYTLPPSREEMTKNQVSLTCLV | |
| KGFYPSDIAVEWESNGQPENNYKTTPPVLDSDGS | |
| FFLYSKLTVDKSRWQQGNVFSCSVMHEALHNHYT | |
| QKSLSLSPGK. | |

A variant of the preceding humanized 2H7 mAb is 2H7v.31 having the same L chain sequence as SEQ ID NO: 13 above, with the H chain amino acid sequence:

| | |
|---|---|
| EVQLVESGGGLVQPGGSLRLSCAASGYTFTSYNM | (SEQ ID NO: 15) |
| HWVRQAPGKGLEWVGAIYPGNGDTSYNQKFKGRF | |
| TISVDKSKNTLYLQMNSLRAEDTAVYYCARVVYY | |
| SNSYWYFDVWGQGTLVTVSSASTKGPSVFPLAPS | |
| SKSTSGGTAALGCLVKDYFPEPVTVSWNSGALTS | |
| GVHTFPAVLQSSGLYSLSSVVTVPSSSLGTQTYI | |
| CNVNHKPSNTKVDKKVEPKSCDKTHTCPPCPAPE | |
| LLGGPSVFLFPPKPKDTLMISRTPEVTCVVVDVS | |
| HEDPEVKFNWYVDGVEVHNAKTPREEQYNATYRV | |
| VSVLTVLHQDWLNGKEYKCKVSNKALPAPIAATI | |
| SKAKGQPREPQVYTLPPSREEMTKNQVSLTCLVK | |
| GFYPSDIAVEWESNGQPENNYKTTPPVLDSDGSF | |
| FLYSKLTVDKSRWQQGNVFSCSVMHEALHNHYTQ | |
| KSLSLSPGK. | |

Another variant is 2H7.v138 having the H chain amino acid sequence of SEQ ID NO. 26
Another variant of the preceding humanized 2H7 antibody is one that comprises the V_{L} of SEQ ID NO. 25 and V_{H} of SEQ ID NO. 33 of 2H7.v511 [see Table 2]

hu2H7.v511 is a full length IgG1. In one embodiment the antibody comprises the 2H7.v511 Light Chain (SEQ ID NO.26)

DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

And the 2H7.v511 Heavy Chain (SEQ ID NO. 34)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSYRYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHY
TQKSLSLSPGK

The V region of all other variants based on version 16 will have the amino acid sequences of v16 except at the positions of amino acid substitutions which are indicated in Table 1 below. Unless otherwise indicated, the 2H7 variants will have the same L chain as that of v16. Humanized antibody 2H7v. 16 is also referred to as rhuMAb2H7, PRO70769, or Ocrelizumab.

TABLE 1

| 2H7 | Light chain | Heavy chain | |
|---|---|---|---|
| version | (V_{L}) changes | (V_{H}) changes | Fc changes |
| 16 for | | | — |
| reference | | | |
| 31 | — | — | S298A, E333A, K334A |
| 73 | M32L | N100A | |
| 75 | M32L | N100A | S298A, E333A, K334A |
| 96 | S92A | D56A, N100A | |
| 114 | M32L, S92A | D56A, N100A | S298A, E333A, K334A |
| 115 | M32L, S92A | D56A, N100A | S298A, E333A, K334A, |
| | | | E356D, M358L |
| 116 | M32L, S92A | D56A, N100A | S298A, K334A, K322A |
| 138 | M32L, S92A | D56A, N100A | S298A, E333A, K334A, |
| | | | K326A |
| 477 | M32L, S92A | D56A, N100A | S298A, E333A, K334A, |
| | | | K326A, N434W |
| 375 | — | — | K334L |
| 511 | 32L, S92A | D56A, N100Y, | S298A, E333A, |
| | | S100aR | K334A, K326A, |

TABLE 2

| | V_{L} | V_{H} | Full L chain | Full H chain |
|---|---|---|---|---|
| 2H7 version | SEQ ID NO. | SEQ ID NO. | SEQ ID NO. | SEQ ID NO. |
| 16 | 2 | 8 | 13 | 14 |
| 31 | 2 | 8 | 13 | 15 |
| 73 | 16 | 17 | 18 | 19 |
| 75 | 16 | 17 | 18 | 20 |
| 96 | 21 | 22 | 23 | 24 |
| 114 | 25 | 22 | 26 | 27 |
| 115 | 25 | 22 | 26 | 28 |
| 116 | 25 | 22 | 26 | 29 |
| 138 | 25 | 22 | 26 | 30 |
| 477 | 25 | 22 | 26 | 31 |
| 375 | 2 | 8 | 13 | 32 |
| 511 | 25 | 33 | 26 | 34 |

Residue numbering is according to Kabat et al., Sequences of Immunological Interest. 5th Ed. Public Health Service, National Institutes of Health, Bethesda, Md. (1991), with insertions shown as a, b, c, d, and e, and gaps shown as dashes in the sequence figures. In the CD20 binding antibodies that comprise Fc region, the C-terminals lysine (residue 447 according to the EU numbering system) of the Fc region may be removed, for example, during purification of the Ab or by recombinant engineering the nucleic acid encoding the antibody polypeptide. Accordingly, a humanized 2H7 antibody composition of this invention can comprise antibody with K447, with all K447 removed, or a mixture of antibody with and without the K447 residue.

The N-glycosylation site in IgG is at Asn297 in the CH2 domain. Humanized 2H7 antibody compositions of the present invention include compositions of any of the preceding humanized 2H7 antibodies having a Fc region, wherein about 80-100% (and preferably about 90-99%) of the antibody in the composition comprises a mature core carbohydrate structure which lacks fucose, attached to the Fc region of the glycoprotein. Such compositions were demonstrated herein to exhibit a surprising improvement in binding to FcγRIIIA(F158), which is not as effective as FcγRIIIA (V158) in interacting with human IgG. FcγRIIIA (F158) is more common than FcγRIIIA (V158) in normal, healthy African Americans and Caucasians. See Lehrnbecher et al. Blood 94:4220 (1999). Historically, antibodies produced in Chinese Hamster Ovary Cells (CHO), one of the most commonly used industrial hosts, contain about 2 to 6% in the population that are nonfucosylated. YB2/0 and Lec13, however, can produce antibodies with 78 to 98% nonfucosylated species. Shinkawa et al. J. Bio. Chem. 278 (5), 3466-347 (2003), reported that antibodies produced in YB2/0 and Lec13 cells, which have less FUT8 activity, show significantly increased ADCC activity in vitro. The production of antibodies with reduced fucose content are also described in e.g., Li et al. (GlycoFi) “Optimization of humanized IgGs in glycoengineered Pichia pastoris” in Nature Biology online publication 22 Jan. 2006; Niwa R. et al. Cancer Res. 64(6):2127-2133 (2004); US 2003/0157108 (Presta); U.S. Pat. No. 6,602,684 and US 2003/0175884 (Glycart Biotechnology); US 2004/0093621, US 2004/0110704, US 2004/0132140 (all of Kyowa Hakko Kogyo).

A bispecific humanized 2H7 antibody encompasses an antibody wherein one arm of the antibody has at least the antigen binding region of the H and/or L chain of a humanized 2H7 antibody of the invention, and the other arm has V region binding specificity for a second antigen. In specific embodiments, the second antigen is selected from the group consisting of CD3, CD64, CD32A, CD16, NKG2D or other NK activating ligands.

In certain embodiments, the humanized 2H7 antibody of the invention further comprises amino acid alterations in the IgG Fc and exhibits increased binding affinity for human FcRn over an antibody having wild-type IgG Fc, by at least 60 fold, at least 70 fold, at least 80 fold, more preferably at least 100 fold, preferably at least 125 fold, even more preferably at least 150 fold to about 170 fold.

Antibody Production

Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g., by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plüickthun, Immunol. Revs., 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (C_{H} and C_{L}) sequences for the homologous murine sequences (U.S. Pat. No. 4,816,567; and Morrison, et al., Proc. Natl. Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol, 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

The humanized antibody may be an antibody fragment, such as a Fab, which is optionally conjugated with one or more cytotoxic agent(s) in order to generate an immunoconjugate. Alternatively, the humanized antibody may be an full length antibody, such as an full length IgG1 antibody.

Human Antibodies and Phage Display Methodology

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); U.S. Pat. No. 5,545,807; and WO 97/17852.

Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al. J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

Antibody Fragments

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

Bispecific Antibodies

Bispecific antibodies are antibodies that have binding specificities for at least two different epitopes. Exemplary bispecific antibodies may bind to two different epitopes of the CD20 protein. Other such antibodies may combine a CD20 binding site with a binding site for another protein. Alternatively, an anti-CD20 arm may be combined with an arm which binds to a triggering molecule on a leukocyte such as a T-cell receptor molecule (e.g. CD3), or Fc receptors for IgG (FcγR), such as FcγRI (CD64), FcγRII (CD32) and FcγIII (CD16), or NKG2D or other NK cell activating ligand, so as to focus and localize cellular defense mechanisms to the CD20-expressing cell. Bispecific antibodies may also be used to localize cytotoxic agents to cells which express CD20. These antibodies possess a CD20-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)₂ bispecific antibodies).

WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fcα antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210(1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al. J. Immunol. 147: 60 (1991).

Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

Other Amino Acid Sequence Modifications

Amino acid sequence modification(s) of the CD20 binding antibodies described herein are contemplated. For example, it may be desirable to improve the binding affinity and/or other biological properties of the antibody. Amino acid sequence variants of the anti-CD20 antibody are prepared by introducing appropriate nucleotide changes into the anti-CD20 antibody nucleic acid, or by peptide synthesis. Such modifications include, for example, deletions from, and/or insertions into and/or substitutions of, residues within the amino acid sequences of the anti-CD20 antibody. Any combination of deletion, insertion, and substitution is made to arrive at the final construct, provided that the final construct possesses the desired characteristics. The amino acid changes also may alter post-translational processes of the anti-CD20 antibody, such as changing the number or position of glycosylation sites.

A useful method for identification of certain residues or regions of the anti-CD20 antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with CD20 antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed anti-CD20 antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an anti-CD20 antibody with an N-terminal methionyl residue or the antibody fused to a cytotoxic polypeptide. Other insertional variants of the anti-CD20 antibody molecule include the fusion to the N- or C-terminus of the anti-CD20 antibody to an enzyme (e.g. for ADEPT) or a polypeptide which increases the serum half-life of the antibody.

Another type of variant is an amino acid substitution variant. These variants have at least one amino acid residue in the anti-CD20 antibody molecule replaced by a different residue. The sites of greatest interest for substitutional mutagenesis include the hypervariable regions, but FR alterations are also contemplated. Conservative substitutions are shown in the Table below under the heading of “preferred substitutions”. If such substitutions result in a change in biological activity, then more substantial changes, denominated “exemplary substitutions” in the Table, or as further described below in reference to amino acid classes, may be introduced and the products screened.

TABLE of Amino Acid Substitutions

| Original | Exemplary | Preferred |
|---|---|---|
| Residue | Substitutions | Substitutions |
| Ala (A) | val; leu; ile | Val |
| Arg (R) | lys; gln; asn | Lys |
| Asn (N) | gln; his; asp, lys; arg | Gln |
| Asp (D) | glu; asn | Glu |
| Cys (C) | ser; ala | Ser |
| Gln (Q) | asn; glu | Asn |
| Glu (E) | asp; gln | Asp |
| Gly (G) | ala | Ala |
| His (H) | asn; gln; lys; arg | Arg |
| Ile (I) | leu; val; met; ala; phe; norleucine | Leu |
| Leu (L) | norleucine; ile; val; met; ala; phe | Ile |
| Lys (K) | arg; gln; asn | Arg |
| Met (M) | leu; phe; ile | Leu |
| Phe (F) | leu; val; ile; ala; tyr | Tyr |
| Pro (P) | ala | Ala |
| Ser (S) | thr | Thr |
| Thr (T) | ser | Ser |
| Trp (W) | tyr; phe | Tyr |
| Tyr (Y) | trp; phe; thr; ser | Phe |
| Val (V) | ile; leu; met; phe; ala; norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class.

Any cysteine residue not involved in maintaining the proper conformation of the anti-CD20 antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and human CD20. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-CD20 antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-CD20 antibody.

It may be desirable to modify the antibody of the invention with respect to effector function, e.g. so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement mediated lysis and ADCC capabilities. See Stevenson et al. Anti-Cancer Drug Design 3:219-230 (1989).

To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

Other Antibody Modifications

Other modifications of the antibody are contemplated herein. For example, the antibody may be linked to one of a variety of nonproteinaceous polymers, e.g., polyethylene glycol, polypropylene glycol, polyoxyalkylenes, or copolymers of polyethylene glycol and polypropylene glycol. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

Screening for Antibodies with the Desired Properties

Antibodies with certain biological characteristics may be selected as described in the Experimental Examples.

The growth inhibitory effects of an anti-CD20 antibody of the invention may be assessed by methods known in the art, e.g., using cells which express CD20 either endogenously or following transfection with the CD20 gene. For example, tumor cell lines and CD20-transfected cells may treated with an anti-CD20 monoclonal antibody of the invention at various concentrations for a few days (e.g., 2-7) days and stained with crystal violet or MTT or analyzed by some other colorimetric assay. Another method of measuring proliferation would be by comparing ³H-thymidine uptake by the cells treated in the presence or absence an anti-CD20 antibody of the invention. After antibody treatment, the cells are harvested and the amount of radioactivity incorporated into the DNA quantitated in a scintillation counter. Appropriate positive controls include treatment of a selected cell line with a growth inhibitory antibody known to inhibit growth of that cell line.

To select for antibodies which induce cell death, loss of membrane integrity as indicated by, e.g., propidium iodide (PI), trypan blue or 7AAD uptake may be assessed relative to control. A PI uptake assay can be performed in the absence of complement and immune effector cells. CD20-expressing tumor cells are incubated with medium alone or medium containing of the appropriate monoclonal antibody at e.g., about 10 μg/ml. The cells are incubated for a 3 day time period. Following each treatment, cells are washed and aliquoted into 35 mm strainer-capped 12×75 tubes (1 ml per tube, 3 tubes per treatment group) for removal of cell clumps. Tubes then receive PI (10 μg/ml). Samples may be analyzed using a FACSCAN™ flow cytometer and FACSCONVER™ CellQuest software (Becton Dickinson). Those antibodies which induce statistically significant levels of cell death as determined by PI uptake may be selected as cell death-inducing antibodies.

To screen for antibodies which bind to an epitope on CD20 bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test antibody binds the same site or epitope as an anti-CD20 antibody of the invention. Alternatively, or additionally, epitope mapping can be performed by methods known in the art. For example, the antibody sequence can be mutagenized such as by alanine scanning, to identify contact residues. The mutant antibody is initially tested for binding with polyclonal antibody to ensure proper folding. In a different method, peptides corresponding to different regions of CD20 can be used in competition assays with the test antibodies or with a test antibody and an antibody with a characterized or known epitope.

Vectors, Host Cells and Recombinant Methods

The invention also provides an isolated nucleic acid encoding a humanized 2H7 variant antibody, vectors and host cells comprising the nucleic acid, and recombinant techniques for the production of the antibody.

For recombinant production of the antibody, the nucleic acid encoding it is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

(i) Signal Sequence Component

The humanized 2H7 antibody of this invention may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. For prokaryotic host cells that do not recognize and process the native CD20 binding antibody signal sequence, the signal sequence is substituted by a prokaryotic signal sequence selected, for example, from the group of the alkaline phosphatase, penicillinase, 1 pp, or heat-stable enterotoxin II leaders. For yeast secretion the native signal sequence may be substituted by, e.g., the yeast invertase leader, α factor leader (including Saccharomyces and Kluyveromyces α-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available.

The DNA for such precursor region is ligated in reading frame to DNA encoding the humanized 2H7 antibody.

(ii) Origin of Replication

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells. Generally, the origin of replication component is not needed for mammalian expression vectors (the SV40 origin may typically be used only because it contains the early promoter).

(iii) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the nucleic acid encoding the humanized 2H7 antibody, such as DHFR, thymidine kinase, metallothionein-I and -III, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding the humanized 2H7 antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp1 gene present in the yeast plasmid YRp7 (Stinchcomb et al., Nature, 282:39 (1979)). The trp1 gene provides a selection marker for a mutant strain of yeast lacking the ability to grow in tryptophan, for example, ATCC No. 44076 or PEP4-1. Jones, Genetics, 85:12 (1977). The presence of the trp1 lesion in the yeast host cell genome then provides an effective environment for detecting transformation by growth in the absence of tryptophan. Similarly, Leu2-deficient yeast strains (ATCC 20,622 or 38,626) are complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. Van den Berg, Bio/Technology, 8:135 (1990). Stable multi-copy expression vectors for secretion of mature recombinant human serum albumin by industrial strains of Kluyveromyces have also been disclosed. Fleer et al., Bio/Technology, 9:968-975 (1991).

(iv) Promoter Component

Expression and cloning vectors usually contain a promoter that is recognized by the host organism and is operably linked to the nucleic acid encoding the humanized 2H7 antibody. Promoters suitable for use with prokaryotic hosts include the phoA promoter, β-lactamase and lactose promoter systems, alkaline phosphatase promoter, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the CD20 binding antibody.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoter sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Humanized 2H7 antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the Rous Sarcoma Virus long terminal repeat can be used as the promoter.

(v) Enhancer Element Component

Transcription of a DNA encoding the humanized 2H7 antibody of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the CD20 binding antibody-encoding sequence, but is preferably located at a site 5′ from the promoter.

(vi) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding CD20 binding antibody. One useful transcription termination component is the bovine growth hormone polyadenylation region. See WO94/11026 and the expression vector disclosed therein.

(vii) Selection and Transformation of Host Cells

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

Full length antibody, antibody fragments, and antibody fusion proteins can be produced in bacteria, in particular when glycosylation and Fc effector function are not needed, such as when the therapeutic antibody is conjugated to a cytotoxic agent (e.g., a toxin) and the immunoconjugate by itself shows effectiveness in tumor cell destruction. Full length antibodies have greater half life in circulation. Production in E. coli is faster and more cost efficient. For expression of antibody fragments and polypeptides in bacteria, see, e.g., U.S. Pat. No. 5,648,237 (Carter et. al.), U.S. Pat. No. 5,789,199 (Joly et al.), and U.S. Pat. No. 5,840,523 (Simmons et al.) which describes translation initiation region (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed e.g., in CHO cells.

In addition to prokaryotes, eukaryotic microbes such as filamentous fungi or yeast are suitable cloning or expression hosts for CD20 binding antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated humanized 2H7 antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR(CHO, Urlaub et al., Proc. Natl. Acad. Sci. USA 77:4216 (1980)) or CHO-DP-12 line; mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for CD20 binding antibody production and cultured in conventional nutrient media modified as appropriate for inducing promoters, selecting transformants, or amplifying the genes encoding the desired sequences.

(viii) Culturing the Host Cells

The host cells used to produce the CD20 binding antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

(ix) Purification of Antibody

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10: 163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

Antibody Conjugates

The antibody may be conjugated to a cytotoxic agent such as a toxin or a radioactive isotope. In certain embodiments, the toxin is calicheamicin, a maytansinoid, a dolastatin, auristatin E and analogs or derivatives thereof, are preferable.

Preferred drugs/toxins include DNA damaging agents, inhibitors of microtubule polymerization or depolymerization and antimetabolites. Preferred classes of cytotoxic agents include, for example, the enzyme inhibitors such as dihydrofolate reductase inhibitors, and thymidylate synthase inhibitors, DNA intercalators, DNA cleavers, topoisomerase inhibitors, the anthracycline family of drugs, the vinca drugs, the mitomycins, the bleomycins, the cytotoxic nucleosides, the pteridine family of drugs, diynenes, the podophyllotoxins and differentiation inducers. Particularly useful members of those classes include, for example, methotrexate, methopterin, dichloromethotrexate, 5-fluorouracil, 6-mercaptopurine, cytosine arabinoside, melphalan, leurosine, leurosideine, actinomycin, daunorubicin, doxorubicin, N-(5,5-diacetoxypentyl)doxorubicin, morpholino-doxorubicin, 1-(2-choroehthyl)-1,2-dimethanesulfonyl hydrazide, N⁸-acetyl spermidine, aminopterin methopterin, esperamicin, mitomycin C, mitomycin A, actinomycin, bleomycin, carminomycin, aminopterin, tallysomycin, podophyllotoxin and podophyllotoxin derivatives such as etoposide or etoposide phosphate, vinblastine, vincristine, vindesine, taxol, taxotere, retinoic acid, butyric acid, N⁸-acetyl spermidine, camptothecin, calicheamicin, bryostatins, cephalostatins, ansamitocin, actosin, maytansinoids such as DM-1, maytansine, maytansinol, N-desmethyl-4,5-desepoxymaytansinol, C-19-dechloromaytansinol, C-20-hydroxymaytansinol, C-20-demethoxymaytansinol, C-9-SH maytansinol, C-14-alkoxymethylmaytansinol, C-14-hydroxy or acetyloxymethlmaytansinol, C-15-hydroxy/acetyloxymaytansinol, C-15-methoxymaytansinol, C-18-N-demethylmaytansinol and 4,5-deoxymaytansinol, auristatins such as auristatin E, M, PHE and PE; dolostatins such as dolostatin A, dolostatin B, dolostatin C, dolostatin D, dolostatin E (20-epi and 11-epi), dolostatin G, dolostatin H, dolostatin I, dolostatin 1, dolostatin 2, dolostatin 3, dolostatin 4, dolostatin 5, dolostatin 6, dolostatin 7, dolostatin 8, dolostatin 9, dolostatin 10, deo-dolostatin 10, dolostatin 11, dolostatin 12, dolostatin 13, dolostatin 14, dolostatin 15, dolostatin 16, dolostatin 17, and dolostatin 18; cephalostatins such as cephalostatin 1, cephalostatin 2, cephalostatin 3, cephalostatin 4, cephalostatin 5, cephalostatin 6, cephalostatin 7, 25′-epi-cephalostatin 7,20-epi-cephalostatin 7, cephalostatin 8, cephalostatin 9, cephalostatin 10, cephalostatin 11, cephalostatin 12, cephalostatin 13, cephalostatin 14, cephalostatin 15, cephalostatin 16, cephalostatin 17, cephalostatin 18, and cephalostatin 19.

Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, the disclosures of which are hereby expressly incorporated by reference.

Maytansine and maytansinoids have been conjugated to antibodies specifically binding to tumor cell antigens. Immunoconjugates containing maytansinoids and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al. Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene.

There are many linking groups known in the art for making antibody-maytansinoid conjugates, including, for example, those disclosed in U.S. Pat. No. 5,208,020 or EP Patent 0 425 235 B 1, and Chari et al. Cancer Research 52: 127-131 (1992). The linking groups include disulfide groups, thioether groups, acid labile groups, photolabile groups, peptidase labile groups, or esterase labile groups, as disclosed in the above-identified patents, disulfide and thioether groups being preferred.

Conjugates of the antibody and maytansinoid may be made using a variety of bifunctional protein coupling agents such as N-succinimidyl-3-(2-pyridyldithio)propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio)propionate (SPDP) (Carlsson et al., Biochem. J. 173:723-737 [1978]) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hyrdoxymethyl, the C-15 position modified with a hydroxyl group, and the C-20 position having a hydroxyl group. In a preferred embodiment, the linkage is formed at the C-3 position of maytansinol or a maytansinol analogue.

Another immunoconjugate of interest comprises an CD20 binding antibody conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₂^{I}, α₃^{I}, N-acetyl-γ₁^{I}, PSAG and θ^{I}₁ (Hinman et al. Cancer Research 53: 3336-3342 (1993), Lode et al. Cancer Research 58: 2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

Radioactive Isotopes

For selective destruction of the tumor, the antibody may comprise a highly radioactive atom. A variety of radioactive isotopes are available for the production of radioconjugated anti-CD20 antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for diagnosis, it may comprise a radioactive atom for scintigraphic studies, for example tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, Re¹⁸⁶ Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio)propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al. Cancer Research 52: 127-131 (1992); U.S. Pat. No. 5,208,020) may be used.

Therapeutic Uses

The humanized 2H7CD20 binding antibodies of the invention are useful to treat a number of malignant and non-malignant diseases including CD20 positive cancers such as B cell lymphomas and leukemia, and autoimmune diseases. Stem cells (B-cell progenitors) in bone marrow lack the CD20 antigen, allowing healthy B-cells to regenerate after treatment and return to normal levels within several months. hu2H7.v511 is the preferred antibody to be used in the treatment methods herein.

CD20 positive cancers are those comprising abnormal proliferation of cells that express CD20 on the cell surface. The CD20 positive B cell neoplasms include CD20-positive Hodgkin's disease including lymphocyte predominant Hodgkin's disease (LPHD); non-Hodgkin's lymphoma (NHL); follicular center cell (FCC) lymphomas; acute lymphocytic leukemia (ALL); chronic lymphocytic leukemia (CLL); Hairy cell leukemia.

The term “non-Hodgkin's lymphoma” or “NHL”, as used herein, refers to a cancer of the lymphatic system other than Hodgkin's lymphomas. Hodgkin's lymphomas can generally be distinguished from non-Hodgkin's lymphomas by the presence of Reed-Sternberg cells in Hodgkin's lymphomas and the absence of said cells in non-Hodgkin's lymphomas. Examples of non-Hodgkin's lymphomas encompassed by the term as used herein include any that would be identified as such by one skilled in the art (e.g., an oncologist or pathologist) in accordance with classification schemes known in the art, such as the Revised European-American Lymphoma (REAL) scheme as described in Color Atlas of Clinical Hematology (3rd edition), A. Victor Hoffbrand and John E. Pettit (eds.) (Harcourt Publishers Ltd., 2000). See, in particular, the lists in FIGS. 11, 57, 11.58 and 11.59. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

In specific embodiments, the humanized CD20 binding antibodies and functional fragments thereof are used to treat non-Hodgkin's lymphoma (NHL), lymphocyte predominant Hodgkin's disease (LPHD), small lymphocytic lymphoma (SLL), and chronic lymphocytic leukemia (CLL), including relapses of these conditions.

Indolent lymphoma is a slow-growing, incurable disease in which the average patient survives between six and 10 years following numerous periods of remission and relapse. In one embodiment, the humanized CD20 binding antibodies or functional fragments thereof are used to treat indolent NHL including relapsed indolent NHL and rituximab-refractory indolent NHL. The relapsed indolent NHL patients can be Rituximab responders who have previously received one course of Rituximab and have responded for >6 months.

The present humanized 2H7 antibodies or functional fragments thereof are useful as a single-agent treatment (monotherapy) in, e.g., for relapsed or refractory low-grade or follicular, CD20-positive, B-cell NHL, or can be administered to patients in conjunction with other drugs in a multi-drug regimen.

The humanized 2H7 antibodies or functional fragments of the invention can be used as front-line therapy. The invention also contemplates the use of these antibodies for the treatment of patients with CD20 positive B cell neoplasms that are nonresponsive or have an inadequate response to treatment with any one of the following drugs: rituximab (Genentech); ocrelizumab (Genentech, Inc.); ibritumomab tiuxetan (Zevalin™, Biogen Idec); tositumomab (Bexxar™, GlaxoSmithKline); HuMAX-CD20™ (GenMab); IMMU-106 (which is a humanized anti-CD20 a.k.a. hA20 or 90Y-hLL2, Immunomedics); AME-133 (Applied Molecular Evolution/Eli Lilly); gentuzumab ozogamicin (Mylotarg™, a humanized anti-CD33 antibody, Wyeth/PDL); alemtuzumab (Campath™, an anti-CD52 antibody, Schering Plough/Genzyme); epratuzumab (IMMU-103™, a humanized anti-CD22 antibody, Immunomedics), or have relapsed after treatment with these drugs.

The invention further provides a method of treating CLL patients including those who have failed fludarabine therapy with the humanized 2H7 antibodies of the invention and in specific embodiments, with 2H7.v511 and 2H7.v114.

An “autoimmune disease” herein is a disease or disorder arising from and directed against an individual's own tissues or a co-segregate or manifestation thereof or resulting condition therefrom. Examples of autoimmune diseases or disorders include, but are not limited to arthritis (rheumatoid arthritis such as acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, and ataxic sclerosis, inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), pyoderma gangrenosum, erythema nodosum, primary sclerosing cholangitis, episcleritis), respiratory distress syndrome, including adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, allergic conditions and responses, allergic reaction, eczema including allergic or atopic eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis (including large vessel vasculitis (including polymyalgia rheumatica and giant cell (Takayasu's) arteritis), medium vessel vasculitis (including Kawasaki's disease and polyarteritis nodosa/periarteritis nodosa), microscopic polyarteritis, CNS vasculitis, necrotizing, cutaneous, or hypersensitivity vasculitis, systemic necrotizing vasculitis, and ANCA-associated vasculitis, such as Churg-Strauss vasculitis or syndrome (CSS)), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, immune complex nephritis, antibody-mediated nephritis, neuromyelitis optica, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP), post-transfusion purpura (PTP), heparin-induced thrombocytopenia, and autoimmune or immune-mediated thrombocytopenia such as idiopathic thrombocytopenic purpura (ITP) including chronic or acute ITP, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis (e.g., benign monoclonal gammopathy and monoclonal garnmopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, diabetic nephropathy, Dressler's syndrome, alopecia greata, CREST syndrome (calcinosis, Raynaud's phenomenon, esophageal dysmotility, sclerodactyly, and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, infertility due to antispermatozoan antibodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, lymphadenitis, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

In specific embodiments, the humanized 2H7 antibodies and functional fragments thereof are used to treat rheumatoid arthritis and juvenile rheumatoid arthritis, systemic lupus erythematosus (SLE) including lupus nephritis, Wegener's disease, inflammatory bowel disease, ulcerative colitis, idiopathic thrombocytopenic purpura (ITP), thrombotic throbocytopenic purpura (TTP), autoimmune thrombocytopenia, multiple sclerosis including relapsed remitting MS, psoriasis, IgA nephropathy, IgM polyneuropathies, myasthenia gravis, ANCA associated vasculitis, diabetes mellitus, Reynaud's syndrome, Sjogren's syndrome, Neuromyelitis Optica (NMO) and glomerulonephritis.

“Treating” or “treatment” or “alleviation” refers to therapeutic treatment wherein the object is to slow down (lessen) if not cure the targeted pathologic condition or disorder or prevent recurrence of the condition. A subject is successfully “treated” for an autoimmune disease or a CD20 positive B cell malignancy if, after receiving a therapeutic amount of a humanized CD20 binding antibody of the invention according to the methods of the present invention, the subject shows observable and/or measurable reduction in or absence of one or more signs and symptoms of the particular disease. For example, for cancer, significant reduction in the number of cancer cells or absence of the cancer cells; reduction in the tumor size; inhibition (i.e., slow to some extent and preferably stop) of tumor metastasis; inhibition, to some extent, of tumor growth; increase in length of remission, and/or relief to some extent, one or more of the symptoms associated with the specific cancer; reduced morbidity and mortality, and improvement in quality of life issues. Reduction of the signs or symptoms of a disease may also be felt by the patient. Treatment can achieve a complete response, defined as disappearance of all signs of cancer, or a partial response, wherein the size of the tumor is decreased, preferably by more than 50 percent, more preferably by 75%. A patient is also considered treated if the patient experiences stable disease. In one criterion, the h2H7 antibodies of the invention achieve >95% peripheral blood B cell depletion and the B cells return to 25% of baseline. In preferred embodiments, treatment with the antibodies of the invention is effective to result in the cancer patients being progression-free in the cancer 4 months after treatment, preferably 6 months, more preferably one year, even more preferably 2 or more years post treatment. These parameters for assessing successful treatment and improvement in the disease are readily measurable by routine procedures familiar to a physician of appropriate skill in the art.

A “therapeutically effective amount” refers to an amount of an antibody or a drug effective to “treat” a disease or disorder in a subject. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See preceding definition of “treating”. In the case of an autoimmune disease, the therapeutically effective amount of the antibody or other drug is effective to reduce the signs and symptoms of the disease.

The parameters for assessing efficacy or success of treatment of the neoplasm will be known to the physician of skill in the appropriate disease. Generally, the physician of skill will look for reduction in the signs and symptoms of the specific disease. Parameters can include median time to disease progression, time in remission, stable disease.

The following references describe lymphomas and CLL, their diagnoses, treatment and standard medical procedures for measuring treatment efficacy. Canellos G P, Lister, T A, Sklar J L: The Lymphomas. W.B. Saunders Company, Philadelphia, 1998; van Besien K and Cabanillas, F: Clinical Manifestations, Staging and Treatment of Non-Hodgkin's Lymphoma, Chap. 70, pp 1293-1338, in: Hematology, Basic Principles and Practice, 3rd ed. Hoffman et al. (editors). Churchill Livingstone, Philadelphia, 2000; and Rai, K and Patel, D:Chronic Lymphocytic Leukemia, Chap. 72, pp 1350-1362, in: Hematology, Basic Principles and Practice, 3rd ed. Hoffman et al. (editors). Churchill Livingstone, Philadelphia, 2000.

The parameters for assessing efficacy or success of treatment of an autoimmune or autoimmune related disease will be known to the physician of skill in the appropriate disease. Generally, the physician of skill will look for reduction in the signs and symptoms of the specific disease. The following are by way of examples.

In one embodiment, the humanized 2H7 antibodies and specifically hu2H7.v511 and functional fragments thereof are used to treat rheumatoid arthritis.

RA is a debilitating autoimmune disease that affects more than two million Americans and hinders the daily activities of sufferers. RA occurs when the body's own immune system inappropriately attacks joint tissue and causes chronic inflammation that destroys healthy tissue and damage within the joints. Symptoms include inflammation of the joints, swelling, stiffness, and pain. Additionally, since RA is a systemic disease, it can have effects in other tissues such as the lungs, eyes and bone marrow. There is no known cure. Treatments include a variety of steroidal and non-steroidal anti-inflammatory drugs, immunosuppressive agents, disease-modifying anti-rheumatic drugs (DMARDs), and biologics. However, many patients continue to have an inadequate response to treatment.

The antibodies can be used as first-line therapy in patients with early RA (i.e., methotrexate (MTX) naive) and as monotherapy, or in combination with or following, e.g., MTX or cyclophosphamide. Or, the antibodies can be used in treatment as second-line therapy for patients who were DMARD and/or MTX refractory, and as monotherapy or in combination with, e.g., MTX. The humanized CD20 binding antibodies are useful to prevent and control joint damage, delay structural damage, decrease pain associated with inflammation in RA, and generally reduce the signs and symptoms in moderate to severe RA. The RA patient can be treated with the humanized CD20 antibody prior to, after or together with treatment with other drugs used in treating RA (see combination therapy below). In one embodiment, patients who had previously failed disease-modifying antirheumatic drugs and/or had an inadequate response to methotrexate alone are treated with a humanized CD20 binding antibody of the invention. In one embodiment of this treatment, the patients are in a 17-day treatment regimen receiving humanized CD20 binding antibody alone (1 g i.v. infusions on days 1 and 15); CD20 binding antibody plus cyclophosphamide (750 mg i.v. infusion days 3 and 17); or CD20 binding antibody plus methotrexate.

Because the body produces tumor necrosis factor alpha (TNFα) during RA, TNFα inhibitors have been used for therapy of that disease. However, TNFα inhibitors such as Etanercept (ENBREL®), Infliximab (REMICADE®) and Adalimumab (HUMIRA™) can produce negative side effects such as infection, heart failure and demyelination. Therefore, in one embodiment, the humanized CD20 binding antibodies or biologically functional fragments thereof are useful, for example as first-line therapy, to treat RA patients to reduce the risk of these negative side effects experienced with TNFα inhibitor drugs or to treat patients considered to be prone to experience a toxicity, e.g. cardiac toxicity. The humanized CD20 binding antibodies or biologically functional fragments thereof are also useful in a method of treating a subject suffering from RA who has been treated with a TNFα-inhibitor but is nonresponsive, has an inadequate response to the TNFα-inhibitor (TNF-IR patients), or has a relapse of disease after some time of response, or determined to be one who is unlikely to respond to therapy with a TNFα-inhibitor. In one embodiment the TNF-IR are treated with a low dose such as below 100 mg, prior to treatment with a TNFα inhibitor.

One method of evaluating treatment efficacy in RA is based on American College of Rheumatology (ACR) criteria, which measures the percentage of improvement in tender and swollen joints, among other things. The RA patient can be scored at for example, ACR 20 (20 percent improvement) compared with no antibody treatment (e.g., baseline before treatment) or treatment with placebo. Other ways of evaluating the efficacy of antibody treatment include X-ray scoring such as the Sharp X-ray score used to score structural damage such as bone erosion and joint space narrowing. Patients can also be evaluated for the prevention of or improvement in disability based on Health Assessment Questionnaire [HAQ] score, AIMS score, SF-36 at time periods during or after treatment. The ACR 20 criteria may include 20% improvement in both tender (painful) joint count and swollen joint count plus a 20% improvement in at least 3 of 5 additional measures:
1. patient's pain assessment by visual analog scale (VAS),
2. patient's global assessment of disease activity (VAS),
3. physician's global assessment of disease activity (VAS),
4. patient's self-assessed disability measured by the Health Assessment Questionnaire, and
5. acute phase reactants, CRP or ESR.
   The ACR 50 and 70 are defined analogously. Preferably, the patient is administered an amount of a CD20 binding antibody of the invention effective to achieve at least a score of ACR 20, preferably at least ACR 30, more preferably at least ACR50, even more preferably at least ACR70, most preferably at least ACR 75 and higher.

Psoriatic arthritis has unique and distinct radiographic features. For psoriatic arthritis, joint erosion and joint space narrowing can be evaluated by the Sharp score as well. The humanized CD20 binding antibodies of the invention can be used to prevent the joint damage as well as reduce disease signs and symptoms of the disorder.

Yet another aspect of the invention is a method of treating SLE or lupus nephritis by administering to a subject suffering from the disorder, a therapeutically effective amount of a humanized CD20 binding antibody of the invention. SLEDAI scores provide a numerical quantitation of disease activity. The SLEDAI is a weighted index of 24 clinical and laboratory parameters known to correlate with disease activity, with a numerical range of 0-103. see Bryan Gescuk & John Davis, “Novel therapeutic agent for systemic lupus erythematosus” in Current Opinion in Rheumatology 2002, 14:515-521. Other scoring methods include BILAG scoring. Antibodies to double-stranded DNA are believed to cause renal flares and other manifestations of lupus. Patients undergoing antibody treatment can be monitored for time to renal flare, which is defined as a significant, reproducible increase in serum creatinine, urine protein or blood in the urine. Alternatively or in addition, patients can be monitored for levels of antinuclear antibodies and antibodies to double-stranded DNA. Treatments for SLE include high-dose corticosteroids and/or cyclophosphamide (HDCC). Herein, a successful treatment of lupus would reduce flare i.e., reduce the severity and/or time to the next flare.

Spondyloarthropathies are a group of disorders of the joints, including ankylosing spondylitis, psoriatic arthritis and Crohn's disease. Treatment success can be determined by validated patient and physician global assessment measuring tools.

With regard to vasculitis, approximately 75% of the patients with systemic vasculitides have anti-neutrophil cytoplasmic antibody and cluster into one of three conditions affecting small/medium sized vessels: Wegener's granulomatosus (WG), microscopic polyangiitis (MPA) and Churg Strauss syndrome (CSS), collectively known as ANCA associated vasculitis (AAV).

Treatment efficacy for psoriasis is assessed by monitoring changes in clinical signs and symptoms of the disease including Physician's Global Assessment (PGA) changes and Psoriasis Area and Severity Index (PASI) scores, Psoriasis Symptom Assessment (PSA), compared with the baseline condition. The psoriasis patient treated with a humanized CD20 binding antibody of the invention such as hu2H7.v511 can be measured periodically throughout treatment on the Visual analog scale used to indicate the degree of itching experienced at specific time points.

Patients may experience an infusion reaction or infusion-related symptoms with their first infusion of a therapeutic antibody. These symptoms vary in severity and generally are reversible with medical intervention. These symptoms include but are not limited to, flu-like fever, chills/rigors, nausea, urticaria, headache, bronchospasm, angioedema. It would be desirable for the disease treatment methods of the present invention to minimize infusion reactions. To alleviate or minimize such adverse events, the patient may receive an initial conditioning or tolerizing dose(s) of the antibody followed by a therapeutically effective dose. The conditioning dose(s) will be lower than the therapeutically effective dose to condition the patient to tolerate higher dosages.

Dosing

Depending on the indication to be treated and factors relevant to the dosing that a physician of skill in the field would be familiar with, the antibodies of the invention will be administered at a dosage that is efficacious for the treatment of that indication while minimizing toxicity and side effects. The desired dosage may depend on the disease and disease severity, stage of the disease, level of B cell modulation desired, and other factors familiar to the physician of skill in the art.

For treatment of an autoimmune disease, it may be desirable to modulate the extent of B cell depletion depending on the disease and/or the severity of the condition in the individual patient, by adjusting the dosage of humanized 2H7 antibody. B cell depletion can but does not have to be complete. Or, total B cell depletion may be desired in initial treatment but in subsequent treatments, the dosage may be adjusted to achieve only partial depletion. In one embodiment, the B cell depletion is at least 20%, i.e., 80% or less of CD20 positive B cells remain as compared to the baseline level before treatment. In other embodiments, B cell depletion is 25%, 30%, 40%, 50%, 60%, 70% or greater. Preferably, the B cell depletion is sufficient to halt progression of the disease, more preferably to alleviate the signs and symptoms of the particular disease under treatment, even more preferably to cure the disease.

The Genentech and Biogen Idec clinical investigations have evaluated the therapeutic effectiveness of treatment of autoimmune diseases using doses of anti-CD20 (hu2H7.v16 and Rituximab) ranging from as low as 10 mg up to a dose of 1 g (see under Background section for Rituximab studies; and WO 04/056312, Example 16). In general, the antibodies were administered in these clinical investigations in two doses, spaced about two weeks apart. Examples of regimens studied in the clinical investigations include, for humanized CD20 antibody 2H7.v16 in rheumatoid arthritis at 2×10 mg (means 2 doses at 10 mg per dose; total dose of ˜10.1 mg/m² for a 70 kg, 67 inch tall patient), 2×50 mg (total dose of 55 mg/m² for a 70 kg, 67 in tall patient), 2×200 mg (total dose of 220 mg/m² for a 70 kg, 67 in tall patient), 2×500 mg (total dose of ˜550 mg/m2 for a 70 kg, 67 in tall patient) and 2×1000 mg (total dose of ˜1100 mg/m2 for a 70 kg, 67 in tall patient); and for Rituxan, 2×500 mg (total dose of ˜550 mg/m2 for a 70 kg, 67 in tall patient), 2×1000 mg (total dose of ˜1100 mg/m2 for a 70 kg, 67 in tall patient). At each of these doses, substantial depletion of circulating B-lymphocytes was observed following the administration of the first dose of the antibody. At present, a dose range from 10 mg to 2000 mg either as single or dual intravenous infusions have also been explored.

In the present methods of treating autoimmune diseases and of depleting B cells in a patient having an autoimmune disease, in one embodiment, the patient is administered humanized 2H7.v511 antibody at a flat dose in the range of 0.1 mg to 1000 mg. We have found that at flat doses of less than 300 mg, even at 10 mg, substantial B cell depletion is achieved. Thus, in the present B cell depletion and treatment methods in different embodiments, hu2H7.v511 antibody is administered at dosages of 0.1, 0.5, 1, 5, 10, 15, 20 25, 30, 40, 50, 75, 100, 125, 150, 200, or 250 mg. Lower doses e.g., at 20 mg, 10 mg or lower can be used if partial or short term B cell depletion is the objective. In one embodiment, the present dosages and dosing regimen are used in treating rheumatoid arthritis (RA).

For the treatment of a CD20 positive cancer, it may be desirable to maximize the depletion of the B cells which are the target of the anti-CD20 antibodies of the invention. Thus, for the treatment of a CD20 positive B cell neoplasm, it is desirable that the B cell depletion be sufficient to at least prevent progression of the disease which can be assessed by the physician of skill in the art, e.g., by monitoring tumor growth (size), proliferation of the cancerous cell type, metastasis, other signs and symptoms of the particular cancer. Preferably, the B cell depletion is sufficient to prevent progression of disease for at least 2 months, more preferably 3 months, even more preferably 4 months, more preferably 5 months, even more preferably 6 or more months. In even more preferred embodiments, the B cell depletion is sufficient to increase the time in remission by at least 6 months, more preferably 9 months, more preferably one year, more preferably 2 years, more preferably 3 years, even more preferably 5 or more years. In a most preferred embodiment, the B cell depletion is sufficient to cure the disease. In preferred embodiments, the B cell depletion in a cancer patient is at least about 75% and more preferably, 80%, 85%, 90%, 95%, 99% and even 100% of the baseline level before treatment.

Examples of dosing regimens and dosages of hu2H7 antibodies including v16 and v511 for clinical trials in the treatment of NHL are described under Experimental Examples 18-20 below.

Doses at mg/dose of 50, 75, 100, 125, 150, 200, 250, 300, 350 mg/dose can also be used in maintenance therapy for B cell malignancies such as NHL.

The frequency of dosing can vary depending on several factors. Generally, the patient will generally be administered at least 2 doses of the humanized 2H7CD20 binding antibody, and in different embodiments may receive 2-4, 2-8 doses, 2-10 doses. Typically, the 2 doses are administered within a month, generally 1, 2 or 3 weeks apart. In one treatment regimen for oncology, 8 i.v. infusions at doses between 200 mg/m² and 750 mg/m² is contemplated. In one dosing regimen for RA, patients are administered a humanized antibody such as v16, v114 or v511 at 500 mg×2 every 6 months. Another dosing regimen for RA is 1000 mg×2 every 9 months. Yet a third dosing regimen for RA with v16, v114 or v511 is 10 mg×2 every 6 months. Depending on the level of improvement in the disease or recurrence, further doses can be administered over the course of the disease or as disease maintenance therapy.

Patients having an autoimmune disease or a B cell malignancy for whom one or more current therapies were ineffective, poorly tolerated, or contraindicated can be treated using any of the dosing regimens of the present invention. For example, the invention contemplates the present treatment methods for RA patients who have had an inadequate response to tumor necrosis factor (TNF) inhibitor therapies or to disease-modifying anti-rheumatic drugs (DMARD) therapy.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Routes of Administration

The humanized 2H7 antibodies are administered to a human patient in accord with known methods, such as by intravenous administration, e.g., as a bolus or by continuous infusion over a period of time, by subcutaneous, intramuscular, intraperitoneal, intracerobrospinal, intra-articular, intrasynovial, intrathecal, or inhalation routes, generally by intravenous or subcutaneous administration.

Generally, for oncology indications, the humanized 2H7 antibody formulation is administered by intravenous route such as by infusion. In on embodiment, the humanized 2H7 antibody is administered by intravenous infusion with 0.9% sodium chloride solution as an infusion vehicle. The antibody formulation is administered via intravenous infusion in the Phase I/II clinical trial in rheumatoid arthritis (see Example 20).

In another embodiment, the humanized 2H7 antibody is administered by subcutaneous injection, in particular.

Combination Therapy

In treating the B cell neoplasms described above, the patient can be treated with the humanized 2H7 antibodies of the present invention in conjunction with one or more therapeutic agents such as a chemotherapeutic agent in a multidrug regimen. The humanized 2H7 antibody can be administered concurrently, sequentially, or alternating with the chemotherapeutic agent, or after non-responsiveness with other therapy. Standard chemotherapy for lymphoma treatment may include cyclophosphamide, cytarabine, melphalan and mitoxantrone plus melphalan. CHOP is one of the most common chemotherapy regimens for treating Non-Hodgkin's lymphoma. The following are the drugs used in the CHOP regimen: cyclophosphamide (brand names cytoxan, neosar); adriamycin (doxorubicin/hydroxydoxorubicin); vincristine (Oncovin); and prednisolone (sometimes called Deltasone or Orasone). In particular embodiments, the CD20 binding antibody is administered to a patient in need thereof in combination with one or more of the following chemotherapeutic agents of doxorubicin, cyclophosphamide, vincristine and prednisolone. In a specific embodiment, a patient suffering from a lymphoma (such as a non-Hodgkin's lymphoma) is treated with a humanized 2H7 antibody of the present invention in conjunction with CHOP (cyclophosphamide, doxorubicin, vincristine and prednisone) therapy. In another embodiment, the cancer patient can be treated with a humanized 2H7CD20 binding antibody of the invention in combination with CVP (cyclophosphamide, vincristine, and prednisone) chemotherapy. In a specific embodiment, the patient suffering from CD20-positive NHL is administered humanized 2H7.v511 or v114 in conjunction with CVP, for example, every 3 weeks for 8 cycles. In a specific embodiment of the treatment of CLL, the hu2H7.v511 antibody is administered in conjunction with chemotherapy with one or both of fludarabine and cytoxan.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; TLK 286 (TELCYTA™); acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; bisphosphonates, such as clodronate; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)) and anthracyclines such as annamycin, AD 32, alcarubicin, daunorubicin, dexrazoxane, DX-52-1, epirubicin, GPX-100, idarubicin, KRN5500, menogaril, dynemicin, including dynemicin A, an esperamicin, neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin, liposomal doxorubicin, and deoxydoxorubicin), esorubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; folic acid analogues such as denopterin, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenisher such as folinic acid (leucovorin); aceglatone; anti-folate anti-neoplastic agents such as ALIMTA®, LY231514 pemetrexed, dihydrofolate reductase inhibitors such as methotrexate, anti-metabolites such as 5-fluorouracil (5-FU) and its prodrugs such as UFT, S-1 and capecitabine, and thymidylate synthase inhibitors and glycinamide ribonucleotide formyltransferase inhibitors such as raltitrexed (TOMUDEX^{RM}, TDX); inhibitors of dihydropyrimidine dehydrogenase such as eniluracil; aldophosphamide glycoside; aminolevulinic acid; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); cyclophosphamide; thiotepa; taxoids and taxanes, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; gemcitabine (GEMZAR®); 6-thioguanine; mercaptopurine; platinum; platinum analogs or platinum-based analogs such as cisplatin, oxaliplatin and carboplatin; vinblastine (VELBAN®); etoposide (VP-16); ifosfamide; mitoxantrone; vincristine (ONCOVIN®); vinca alkaloid; vinorelbine (NAVELBINE®); novantrone; edatrexate; daunomycin; aminopterin; xeloda; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

Additionally, the hu2H7 antibodies and functional fragments thereof can be used to treat a CD20 expressing B cell neoplasm (e.g, NHL) in conjunction with an anti-tumor angiogenesis agent such as a Vascular Endothelial Growth Factor (VEGF) antagonist. An “anti-angiogenesis agent” or “angiogenesis inhibitor” refers to a small molecular weight substance, a polynucleotide, a polypeptide, an isolated protein, a recombinant protein, an antibody, or conjugates or fusion proteins thereof, that inhibits angiogenesis, vasculogenesis, or undesirable vascular permeability, either directly or indirectly. For example, an anti-angiogenesis agent is an antibody or other antagonist to an angiogenic agent as defined above, e.g., antibodies to VEGF, antibodies to VEGF receptors, small molecules that block VEGF receptor signaling (e.g., PTK787/ZK2284, SU6668). A “VEGF antagonist” refers to a molecule capable of neutralizing, blocking, inhibiting, abrogating, reducing or interfering with VEGF activities including its binding to one or more VEGF receptors. In one embodiment, a patient suffering from such a B cell neoplasm is treated with 2H7.v511 or 2H7.v114 in conjuction with Avastin® (bevacizumab; Genentech). The anti-VEGF antibody “bevacizumab (BV)”, also known as “rhuMAb VEGF” or “Avastin®”, is a recombinant humanized anti-VEGF monoclonal antibody generated according to Presta et al. Cancer Res. 57:4593-4599 (1997).

The hu2H7 antibodies and functional fragments thereof, and in specific embodiments, 2H7.v511 and 2H7.v114, are also useful in a method of treating a CD20 expressing B cell neoplasm in conjunction with a member of the TNF family of cytokines such as Apo-2 ligand (Apo2L) also referred to as TRAIL. The full length native sequence human Apo-2 ligand is a 281 amino acid long, Type II transmembrane protein of the tumor necrosis factor family of cytokines. Soluble forms of the Apo-2 ligand, such as those comprising an extracellular domain (ECD) or portions thereof, have been found to have various activities, including apoptotic activity in mammalian cancer cells. Apo2L/TRAIL (described in WO 97/01633 and WO 97/25428) is a soluble human protein which is a fragment of the ECD, comprising amino acid 114-281 of the full length Apo-2L protein.

In treating the autoimmune diseases or autoimmune related conditions described above, the patient can be treated with one or more hu2H7 antibodies such as hu2H7.v511, in conjunction with a second therapeutic agent, such as an immunosuppressive agent, such as in a multi drug regimen. The hu2H7 antibody can be administered concurrently, sequentially or alternating with the immunosuppressive agent or upon non-responsiveness with other therapy. The immunosuppressive agent can be administered at the same or lesser dosages than as set forth in the art. The preferred adjunct immunosuppressive agent will depend on many factors, including the type of disorder being treated as well as the patient's history.

“Immunosuppressive agent” as used herein for adjunct therapy refers to substances that act to suppress or mask the immune system of a patient. Such agents would include substances that suppress cytokine production, down regulate or suppress self-antigen expression, or mask the MHC antigens. Examples of such agents include steroids such as glucocorticosteroids, e.g., prednisone, methylprednisolone, and dexamethasone; 2-amino-6-aryl-5-substituted pyrimidines (see U.S. Pat. No. 4,665,077), azathioprine (or cyclophosphamide, if there is an adverse reaction to azathioprine); bromocryptine; glutaraldehyde (which masks the MHC antigens, as described in U.S. Pat. No. 4,120,649); anti-idiotypic antibodies for MHC antigens and MHC fragments; cyclosporin A; cytokine or cytokine receptor antagonists including anti-interferon-γ, -β, or -α antibodies; anti-tumor necrosis factor-α antibodies; anti-tumor necrosis factor-β antibodies; anti-interleukin-2 antibodies and anti-IL-2 receptor antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; TGF-β; streptodornase; RNA or DNA from the host; FK506; RS-61443; deoxyspergualin; rapamycin; T-cell receptor (U.S. Pat. No. 5,114,721); T-cell receptor fragments (Offner et al., Science 251:430-432 (1991); WO 90/11294; and WO 91/01133); and T cell receptor antibodies (EP 340,109) such as T10B9.

For the treatment of rheumatoid arthritis, the patient can be treated with a CD20 binding antibody (such as rituximab or ocrelizumab or variant thereof) in conjunction with any one or more of the following drugs: DMARDS (disease-modifying anti-rheumatic drugs (e.g., methotrexate), NSAI or NSAID (non-steroidal anti-inflammatory drugs), immunosuppressants (e.g., azathioprine; mycophenolate mofetil (CellCept®; Roche)), analgesics, glucocorticosteroids, cyclophosphamide, HUMIRA™ (adalimumab; Abbott Laboratories), ARAVA® (leflunomide), REMICADE® (infliximab; Centocor Inc., of Malvern, Pa.), ENBREL (etanercept; Immunex, Wash.), ACTEMRA (tocilizumab; Roche, Switzerland), COX-2 inhibitors. DMARDs commonly used in RA are hydroxycloroquine, sulfasalazine, methotrexate, leflunomide, etanercept, infliximab, azathioprine, D-penicillamine, Gold (oral), Gold (intramuscular), minocycline, cyclosporine, Staphylococcal protein A immunoadsorption.

Adalimumab is a human monoclonal antibody that binds to TNFα. Infliximab is a chimeric mouse-human monoclonal antibody that binds to TNFα. It is an immune-suppressing drug prescribed to treat RA and Crohn's disease. Infliximab has been linked to a fatal reactions such as heart failure and infections including tuberculosis as well as demyelination resulting in MS. Actemra (tocilizumab) is a humanized anti-human interleukin-6 (IL-6) receptor.

Etanercept is an “immunoadhesin” fusion protein consisting of the extracellular ligand binding portion of the human 75 kD (p75) tumor necrosis factor receptor (TNFR) linked to the Fc portion of a human IgG1. Etanercept (ENBREL®) is an injectable drug approved in the US for therapy of active RA. Etanercept binds to TNFα and serves to remove most TNFα from joints and blood, thereby preventing TNFα from promoting inflammation and other symptoms of rheumatoid arthritis. The drug has been associated with negative side effects including serious infections and sepsis, nervous system disorders such as multiple sclerosis (MS). See, e.g., www.remicade-infliximab.com/pages/enbrel_embrel.html

For conventional treatment of RA, see, e.g., “Guidelines for the management of rheumatoid arthritis” Arthritis & Rheumatism 46(2): 328-346 (February, 2002). In a specific embodiment, the RA patient is treated with a hu2H7CD20 antibody of the invention in conjunction with methotrexate (MTX). An exemplary dosage of MTX is about 7.5-25 mg/kg/wk. MTX can be administered orally and subcutaneously.

In one example, patients also receive concomitant MTX (10-25 mg/week per oral (p.o.) or parenteral), together with a corticosteroid regimen consisting of methylprednisolone 100 mg i.v. 30 minutes prior to infusions of the CD20 antibody and prednisone 60 mg p.o. on Days 2-7, 30 mg p.o. Days 8-14, returning to baseline dose by Day 16. Patients may also receive folate (5 mg/week) given as either a single dose or as divided daily doses. Patients optionally continue to receive any background corticosteroid (10 mg/d prednisone or equivalent) throughout the treatment period.

For the treatment of ankylosing spondylitis, psoriatic arthritis and Crohn's disease, the patient can be treated with a CD20 binding antibody of the invention in conjunction with, for example, Remicade® (infliximab; from Centocor Inc., of Malvern, Pa.), ENBREL (etanercept; Immunex, Wash.).

Treatments for SLE include combination of the CD20 antibody with high-dose corticosteroids and/or cyclophosphamide (HDCC). Patients suffering from SLE, AAV and NMO can be treated with a 2H7 antibody of the invention in combination with any of the following: corticosteroids, NSAIDs, analgesics, COX-2 inhibitors, glucocorticosteriods, conventional DMARDS (e.g. methotexate, sulphasalazine, hydroxychloroquine, leflunomide), biologic DMARDs such as anti-Blys (e.g., belimumab), anti-IL6R e.g., tocilizumab; CTLA4-Ig (abatacept), (anti-CD22 e.g., epratuzumab), immunosuppressants (e.g., azathioprine; mycophenolate mofetil (CellCept®; Roche)), and cytotoxic agents (e.g., cyclophosphamide).

For the treatment of psoriasis, patients can be administered a humanized 2H7 antibody in conjunction with topical treatments, such as topical steroids, anthralin, calcipotriene, clobetasol, and tazarotene, or with methotrexate, retinoids, cyclosporine, PUVA and UVB therapies. In one embodiment, the psoriasis patient is treated with a humanized 2H7 antibody sequentially or concurrently with cyclosporine.

The hu2H7 antibodies of the invention and in particular, hu2H7.v511 and hu2H7.v114 are useful to treat an autoimmune disease or a B cell neoplasm in conjuction with a BAFF antagonist, either concurrently, sequentially or in alternating regimen. BAFF and BAFF antagonists are defined above. In one embodiment, the BAFF antagonist is an antibody that binds human BR3, which antibody is preferably humanized, human or chimeric. In another embodiment, the BAFF antagonist is a BR3-Fc fusion protein. The synergistic effects of anti-CD20 antibody with BAFF antagonist in depleting B cells have been reported (see, e.g., WO 05/000351 supra; Gong et al., (2005), J. Immunol. 174: 817-826).

To minimize toxicity, the traditional systemic therapies can be administered in rotational, sequential, combinatorial, or intermittent treatment regimens, or lower dosage combination regimens with the hu2H7CD20 binding antibody compositions at the present dosages.

Pharmaceutical Formulations

Therapeutic formulations of the hu2H7CD20-binding antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Exemplary hu2H7 antibody formulations are described in WO98/56418, expressly incorporated herein by reference. Another formulation is a liquid multidose formulation comprising the hu2H7 antibody at 40 mg/mL, 25 mM acetate, 150 mM trehalose, 0.9% benzyl alcohol, 0.02% polysorbate 20 at pH 5.0 that has a minimum shelf life of two years storage at 2-8° C. Another anti-CD20 antibody formulation of interest comprises 10 mg/mL antibody in 9.0 mg/mL sodium chloride, 7.35 mg/mL sodium citrate dihydrate, 0.7 mg/mL polysorbate 80, and Sterile Water for Injection, pH 6.5. Yet another aqueous pharmaceutical formulation comprises 10-30 mM sodium acetate from about pH 4.8 to about pH 5.5, preferably at pH5.5, polysorbate as a surfactant in a an amount of about 0.01-0.1% v/v, trehalose at an amount of about 2-10% w/v, and benzyl alcohol as a preservative (U.S. Pat. No. 6,171,586). Lyophilized formulations adapted for subcutaneous administration are described in WO97/04801. Such lyophilized formulations may be reconstituted with a suitable diluent to a high protein concentration and the reconstituted formulation may be administered subcutaneously to the mammal to be treated herein.

In a specific embodiment, one IV formulation of humanized 2H7 v16 is: 30 mg/ml antibody in 20 mM sodium acetate, 4% trehalose dihydrate, 0.02% polysorbate 20 (Tween 20™), pH 5.3. This IV formulation can be administered for oncology indications such as NHL, as well as for rheumatoid arthritis. One formulation for the humanized 2H7.v511 variant is 15-30 mg/ml antibody, preferably 20 mg/mL antibody, in 10 mM histidine sulfate, 60 mg/ml sucrose (6%), 0.2 mg/ml polysorbate 20 (0.02%), and Sterile Water for Injection, at pH5.8. In yet another embodiment, the formulation for 2H7 variants and in particular 2H7.v511 is 20 mg/ml 2H7, 20 mM sodium acetate, 4% trehalose dihydrate, 0.02% polysorbate 20, pH 5.5, for intravenous administration. This formulation can be used for single i.v. infusions. One formulation for 2H7.v 114 is antibody at 15-25 mg/ml, preferably 20 mg/ml, in 20 mM Sodium Acetate, 240 mM (8%) trehalose dihydrate, 0.02% Polysorbate 20, pH 5.3.

The formulation herein may also contain more than one active compound as necessary for the particular indication being treated, preferably those with complementary activities that do not adversely affect each other. For example, it may be desirable to further provide a cytotoxic agent, chemotherapeutic agent, cytokine or immunosuppressive agent (e.g. one which acts on T cells, such as cyclosporin or an antibody that binds T cells, e.g. one which binds LFA-1). The effective amount of such other agents depends on the amount of antibody present in the formulation, the type of disease or disorder or treatment, and other factors discussed above. These are generally used in the same dosages and with administration routes as described herein or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

Articles of Manufacture and Kits

Another embodiment of the invention is an article of manufacture containing materials useful for the treatment of autoimmune diseases and related conditions and CD20 positive cancers such as non-Hodgkin's lymphoma. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). At least one active agent in the composition is a hu2H7 antibody, e.g., hu2H7.v511 of the invention. The label or package insert indicates that the composition is used for treating the particular condition. The label or package insert will further comprise instructions for administering the antibody composition to the patient.

Package insert refers to instructions customarily included in commercial packages of therapeutic products, that contain information about the indications, usage, dosage, administration, contraindications and/or warnings concerning the use of such therapeutic products. In one embodiment, the package insert indicates that the composition is used for treating non-Hodgkins' lymphoma.

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for B-cell killing assays, as a positive control for apoptosis assays, for purification or immunoprecipitation of CD20 from cells. For isolation and purification of CD20, the kit can contain a hu2H7.v511 antibody coupled to beads (e.g., sepharose beads). Kits can be provided which contain the antibodies for detection and quantitation of CD20 in vitro, e.g. in an ELISA or a Western blot. As with the article of manufacture, the kit comprises a container and a label or package insert on or associated with the container. The container holds a composition comprising at least one anti-CD20 antibody of the invention. Additional containers may be included that contain, e.g., diluents and buffers, control antibodies. The label or package insert may provide a description of the composition as well as instructions for the intended in vitro or diagnostic use.

### EXPERIMENTAL EXAMPLES

Materials

Mice: Generation of human CD20 (hCD20)⁺ mice was accomplished through the use of bacterial artificial chromosomes (BAC) incorporating the hCD20 locus. Positive BAC clones were injected into blastocytes derived from FVB mice to generate transgenic (Tg) founder lines that expressed hCD20. See detailed description of generation and characterization of hCD20 Tg⁺ mice in Gong et al. (2005) J. Immunol. 174:817-826. HCD20Tg⁺ mice were subsequently bred with hCD16Tg⁺ mCD16^{-/-} to generate hCD20Tg⁺ mCD16^{-/-}hCD16Tg⁺ mice, which were used in the studies described below.

Antibodies: all antibodies used in FACS analysis were purchased from BD PharMingen.

Equipments/software: FACS analysis was performed using FACScan or FACSCalibur machines, and using CellQuest software purchased from Becton Dickinson.

General Methods

Preparation of single cell suspensions: 50 μl of mouse blood was collected orbitally using EDTA-containing tubes, followed by a red cell lysis using ACK Lysis Buffer (purchased from Biosource). Following their removals, mouse spleens were granulized into single cell suspensions using frosted glass slides. Cell pellet was then washed, resuspended and filtered. 5 ml of cold PBS was used to wash peritoneal cavity of each mouse. Lavage fluid was recovered using 5-ml syringes followed by a centrifugation. Leukocytes from mouse blood, spleen and peritoneum were then washed, and counted before staining for FACS analysis.

FACS staining: approximately 1×10⁶ were stained for the various cell surface makers (see definitions in FACS analysis) in 100 μl of phosphate-buffered-saline plus 1% albumin (Sigma). Following a 30-minute-incubation on ice, stained cells were then washed and resuspended before being subjected for FACS analysis.

FACS analysis: FACS analysis was performed using FACScan or FACSCalibur with CellQuest software. B cells in blood were defined as CD21⁺CD23⁺; peritoneal B cells were defined as CD19⁺; splenic B cells were defined as B220⁺; MZ (marginal zone) B cells were defined as CD21^{high} CD23^{low}; and FO (follicular) B cells were defined as CD21⁺CD23^{high}.

Statistics: data were expressed as the mean±standard error (n=5). Statistical analysis was performed using two-tail unpaired T test.

### Example 1

### Humanization of 2H7 Anti-CD20 Murine Monoclonal Antibody

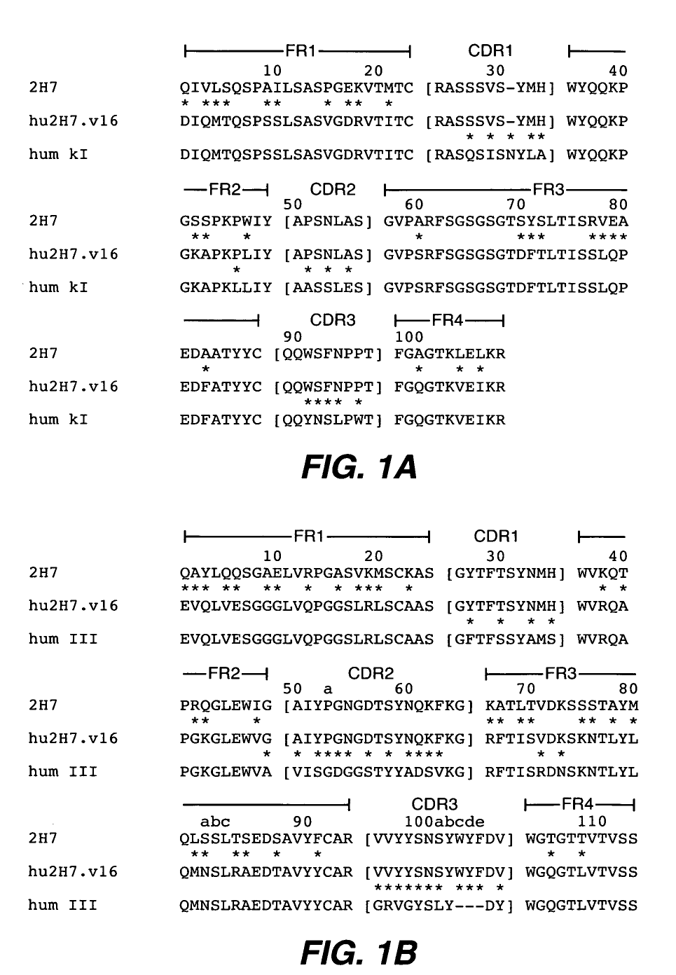

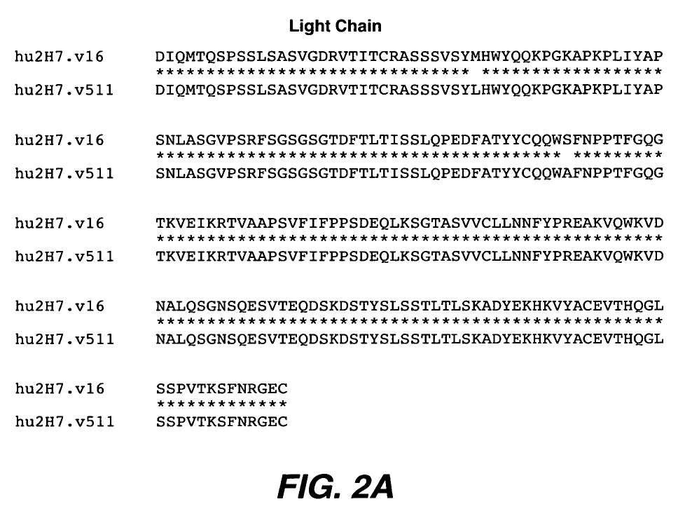

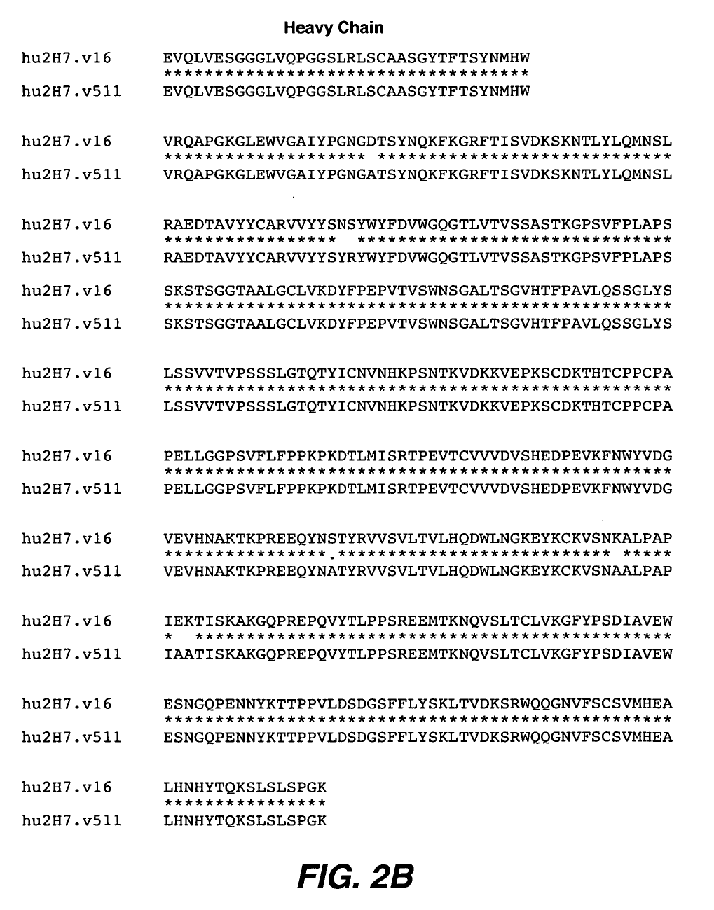

Humanization of the murine anti-human CD20 antibody, 2H7 (also referred to herein as m2H7, m for murine), was carried out in a series of site-directed mutagenesis steps. The murine 2H7 antibody variable region sequences and the chimeric 2H7 with the mouse V and human C have been described, see, e.g., U.S. Pat. Nos. 5,846,818 and 6,204,023. The CDR residues of 2H7 were identified by comparing the amino acid sequence of the murine 2H7 variable domains (disclosed in U.S. Pat. No. 5,846,818) with the sequences of known antibodies (Kabat et al., Sequences of proteins of immunological interest, Ed. 5. Public Health Service, National Institutes of Health, Bethesda, Md. (1991)). The CDR regions for the light and heavy chains were defined based on sequence hypervariability (Kabat et al., supra) and are shown in FIG. 1A and FIG. 1B, respectively. Using synthetic oligonucleotides (Table 1), site-directed mutagenesis (Kunkel, Proc. Natl. Acad. Sci. 82:488-492 (1985)) was used to introduce all six of the murine 2H7CDR regions into a complete human Fab framework corresponding to a consensus sequence V_{κ}I,V_{H}III (V_{L} kappa subgroup I, V_{H} subgroup III) contained on plasmid pVX4 (see FIG. 2 in WO 04/056342 which PCT publication is incorporated herein in its entirety).

The phagemid pVX4 was used for mutagenesis as well as for expression of F(ab)s in E. coli. Based on the phagemid pb0720, a derivative of pB0475 (Cunningham et al., Science 243: 1330-1336 (1989)), pVX4 contains a DNA fragment encoding a humanized consensus κ-subgroup I light chain (V_{L}κI-C_{L}) and a humanized consensus subgroup III heavy chain (V_{H}III-C_{H}1) anti-IFN-α (interferon α) antibody. pVX4 also has an alkaline phosphatase promotor and Shine-Dalgamo sequence both derived from another previously described pUC119-based plasmid, pAK2 (Carter et al., Proc. Natl. Acad. Sci. USA 89: 4285 (1992)). A unique Spe1 restriction site was introduced between the DNA encoding for the F(ab) light and heavy chains. The first 23 amino acids in both anti-IFN-α heavy and light chains are the StII secretion signal sequence (Chang et al., Gene 55: 189-196 (1987)).

To construct the CDR-swap version of 2H7 (2H7.v2), site-directed mutagenesis was performed on a deoxyuridine-containing template of pVX4; all six CDRs of anti-IFN-α were changed to the murine 2H7CDRs. The resulting molecule is referred to as humanized 2H7 version 2 (2H7.v2), or the “CDR-swap version” of 2H7; it has the m2H7CDR residues with the consensus human FR residues shown in FIGS. 1A and 1B. Humanized 2H7.v2 was used for further humanization.

Table 3 shows the oligonucleotide sequence used to create each of the murine 2H7 (m2H7) CDRs in the H and L chain. For example, the CDR-H1 oligonucleotide was used to recreate the m2H7H chain CDR1. CDR-H1, CDR-H2 and CDR-H3 refers to the H chain CDR1, CDR2 and CDR3, respectively; similarly, CDR-L1, CDR-L2 and CDR-L3 refers to each of the L chain CDRs. The substitutions in CDR-H2 were done in two steps with two oligonucleotides, CDR-H2A and CDR-H₂B.

TABLE 3

Oligonucleotide sequences used for construction
of the CDR-swap of murine 2H7 CDRs into a human
framework in pVX4. Residues changed by each ol-
igonucleotide are underlined.

| | |
|---|---|
| Substitution | Oligonucleotide sequence |

| | |
|---|---|
| CDR-H1 | C TAC ACC TTC ACG AGC TAT AAC ATG CAC |
| | TGG GTC CG |
| | (SEQ ID NO. 35) |
| CDR-H2A | G ATT AAT CCT GAC AAC GGC GAC ACG AGC |
| | TAT AAC CAG AAG TTC AAG GGC CG |
| | (SEQ ID NO. 36) |
| CDR-H2B | GAA TGG GTT GCA GCG ATC TAT CCT GGC |
| | AAC GGC GAC AC |
| | (SEQ ID NO. 37) |
| CDR-H3 | AT TAT TGT GCT CGA GTG GTC TAC TAT |
| | AGC AAC AGC TAC TGG TAC TTC GAC GTC |
| | TGG GGT CAA GGA |
| | (SEQ ID NO. 38) |
| CDR-L1 | C TGC ACA GCC AGC TCT TCT GTC AGC TAT |
| | ATG CAT TG |
| | (SEQ ID NO. 39) |
| CDR-L2 | AA CTA CTG ATT TAC GCT CCA TCG AAC |
| | CTC GCG TCT GGA GTC C |
| | (SEQ ID NO. 40) |
| CDR-L3 | TAT TAC TGT CAA CAG TGG AGC TTC AAT |
| | CCG CCC ACA TTT GGA CAG |
| | (SEQ ID NO. 41) |

For comparison with humanized constructs, a plasmid expressing a chimeric 2H7Fab (containing murine V_{L} and V_{H} domains, and human C_{L} and CH₁ domains) was constructed by site-directed mutagenesis (Kunkel, supra) using synthetic oligonucleotides to introduce the murine framework residues into 2H7.v2. The resulting plasmid construct for expression of the chimeric Fab known as 2H7.v6.8, is shown in FIG. 3 in WO 04/056342. Each encoded chain of the Fab has a 23 amino acid StII secretion signal sequence as described for pVX4 above.

Based on a sequence comparison of the murine 2H7 framework residues with the human V_{κ},I,V_{H}III consensus framework (FIGS. 1A and 1B) and previously humanized antibodies (Carter et al., Proc. Natl. Acad. Sci. USA 89:4285-4289 (1992)), several framework mutations were introduced into the 2H7.v2 Fab construct by site-directed mutagenesis. These mutations result in a change of certain human consensus framework residues to those found in the murine 2H7 framework, at sites that might affect CDR conformations or antigen contacts. Version 3 contained V_{H}(R71V, N73K), version 4 contained V_{H}(R71V), version 5 contained V_{H}(R71V, N73K) and V_{L}(L46P), and version 6 contained V_{H}(R71V, N73K) and V_{L}(L46P, L47W).

Humanized and chimeric Fab versions of m2H7 antibody were expressed in E. coli and purified as follows. Plasmids were transformed into E. coli strain XL-1 Blue (Stratagene, San Diego, Calif.) for preparation of double-and single-stranded DNA. For each variant, both light and heavy chains were completely sequenced using the dideoxynucleotide method (Sequenase, U.S. Biochemical Corp.). Plasmids were transformed into E. coli strain 16C9, a derivative of MM294, plated onto LB plates containing 5 μg/ml carbenicillin, and a single colony selected for protein expression. The single colony was grown in 5 ml LB-100 μg/ml carbenicillin for 5-8 h at 37° C. The 5 ml culture was added to 500 ml AP5-100 μg/ml carbenicillin and allowed to grow for 16 h in a 4 L baffled shake flask at 37° C. AP5 media consists of: 1.5 g glucose, 11.0 Hycase SF, 0.6 g yeast extract (certified), 0.19 g anhydrous MgSO₄, 1.07 g NH₄Cl, 3.73 g KCl, 1.2 g NaCl, 120 ml 1 M triethanolamine, pH 7.4, to 1 L water and then sterile filtered through 0.1 μm Sealkeen filter.

Cells were harvested by centrifugation in a 1 L centrifuge bottle (Nalgene) at 3000×g and the supernatant removed. After freezing for 1 h, the pellet was resuspended in 25 ml cold 10 mM MES-10 mM EDTA, pH 5.0 (buffer A). 250 μl of 0.1M PMSF (Sigma) was added to inhibit proteolysis and 3.5 ml of stock 10 mg/ml hen egg white lysozyme (Sigma) was added to aid lysis of the bacterial cell wall. After gentle shaking on ice for 1 h, the sample was centrifuged at 40,000×g for 15 min. The supernatant was brought to 50 ml with buffer A and loaded onto a 2 ml DEAE column equilibrated with buffer A. The flow-through was then applied to a protein G-Sepharose CL-4B (Pharmacia) column (0.5 ml bed volume) equilibrated with buffer A. The column was washed with 10 ml buffer A and eluted with 3 ml 0.3 M glycine, pH 3.0, into 1.25 ml 1 M Tris, pH 8.0. The F(ab) was then buffer exchanged into PBS using a Centricon-30 (Amicon) and concentrated to a final volume of 0.5 ml. SDS-PAGE gels of all F(ab)s were run to ascertain purity and the molecular weight of each variant was verified by electrospray mass spectrometry.

In cell-based ELISA binding assays (described below), the binding of Fabs, including chimeric 2H7Fab, to CD20 was difficult to detect. Therefore, the 2H7Fab versions were reformatted as full-length IgG1 antibodies for assays and further mutagenesis.

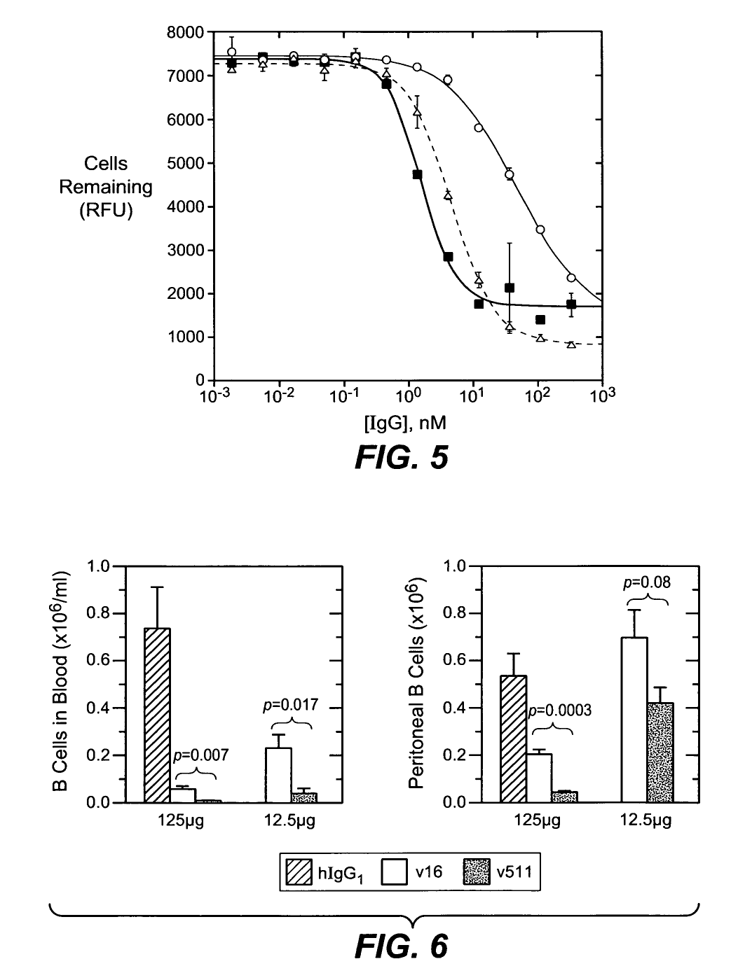

Plasmids for expression of full-length IgG's were constructed by subcloning the V_{L} and V_{H} domains of chimeric 2H7 (v6.8) Fab as well as humanized Fab versions 2 to 6 into previously described pRK vectors for mammalian cell expression (Gorman et al., DNA Prot. Eng. Tech. 2:3-10 (1990)). Briefly, each Fab construct was digested with EcoRV and BlpI to excise a V_{L} fragment, which was cloned into the EcoRV/BlpI sites of plasmid pDR1 (FIG. 4 of WO 04/056312) for expression of the complete light chain (V_{L}-C_{L} domains). Additionally, each Fab construct was digested with PvuII and ApaI to excise a V_{H} fragment, which was cloned into the PvuII/ApaI sites of plasmid pDR2 (FIG. 5 of WO 04/056312) for expression of the complete heavy chain (V_{H}-CH₁-hinge-CH₂—CH₃ domains). For each IgG variant, transient transfections were performed by cotransfecting a light-chain expressing plasmid and a heavy-chain expressing plasmid into an adenovirus-transformed human embryonic kidney cell line, 293 (Graham et al., J. Gen. Virol., 36:59-74, (1977)). Briefly, 293 cells were split on the day prior to transfection, and plated in serum-containing medium. On the following day, double-stranded DNA prepared as a calcium phosphate precipitate was added, followed by pAdVAntage™ DNA (Promega, Madison, Wis.), and cells were incubated overnight at 37° C. Cells were cultured in serum-free medium and harvested after 4 days. Antibodies were purified from culture supernatants using protein A-Sepharose CL-4B, then buffer exchanged into 10 mM sodium succinate, 140 mM NaCl, pH 6.0, and concentrated using a Centricon-10 (Amicon). Protein concentrations were determined by quantitative amino acid analysis.

To measure relative binding affinities to the CD20 antigen, a cell-based ELISA assay was developed. Human B-lymphoblastoid WIL2-S cells (ATCC CRL 8885, American Type Culture Collection, Rockville, Md.) were grown in RPMI 1640 supplemented with 2 mM L-glutamine, 20 mM HEPES, pH 7.2 and 10% heat-inactivated fetal bovine serum in a humidified 5% CO₂ incubator. The cells were washed with PBS containing 1% FBS (assay buffer) and seeded at 250-300,000 cell/well in 96-well round bottom plates (Nunc, Roskilde, Denmark). Two-fold serially diluted standard (15.6-1000 ng/ml of 2H7 v6.8 chimeric IgG) and threefold serially diluted samples (2.7-2000 ng/ml) in assay buffer were added to the plates. The plates were buried in ice and incubated for 45 min. To remove the unbound antibody, 0.1 mL assay buffer were added to the wells. Plates were centrifuged and supernatants were removed. Cells were washed two more times with 0.2 mL assay buffer. Antibody bound to the plates was detected by adding peroxidase conjugated goat anti-human Fc antibody (Jackson ImmunoResearch, West Grove, Pa.) to the plates. After a 45 min incubation, cells were washed as described before. TMB substrate (3,3′,5,5′-tetramethyl benzidine; Kirkegaard & Perry Laboratories, Gaithersburg, Md.) was added to the plates. The reaction was stopped by adding 1 M phosphoric acid. Titration curves were fit with a four-parameter nonlinear regression curve-fitting program (KaleidaGraph, Synergy software, Reading, Pa.). The absorbance at the midpoint of the titration curve (mid-OD) and its corresponding concentration of the standard were determined. Then the concentration of each variant at this mid-OD was determined, and the concentration of the standard was divided by that of each variant. Hence the values are a ratio of the binding of each variant relative to the standard. Standard deviations in relative affinity (equivalent concentration) were generally +/−10% between experiments.

As shown in Table 4, CD20 binding of the CDR-swap variant (v.2) was extremely reduced compared to chimeric 2H7 (v.6.8). However, versions 3 to 6 showed improved binding. To determine the minimum number of mutations that might be required to restore binding affinity to that of chimeric 2H7, additional mutations and combinations of mutations were constructed by site-direct mutagenesis to produce variants 7 to 17 as indicated in Table 5. In particular, these included V_{H} mutations A49G, F67A, I69L, N73K, and L78A; and V_{L} mutations M4L, M331, and F71Y. Versions 16 and 17 showed the best relative binding affinities, within 2-fold of that of the chimeric version, with no significant difference (s.d. =+/−10%) between the two. To minimize the number of mutations, version 16, having only 4 mutations of human framework residues to murine framework residues (Table 5), was therefore chosen as the humanized form for additional characterization.

TABLE 4
Relative binding affinity of humanized 2H7 IgG variants to CD20
compared to chimeric 2H7 using cell-based ELISA. The relative
binding is expressed as the concentration of the chimeric 2H7 over
the concentration of the variant required for equivalent binding; hence
a ratio < 1 indicates weaker affinity for the variant. Standard
deviation in relative affinity determination averaged +/−10%.
Framework substitutions in the variable domains are relative to the
CDR-swap version according to the numbering system
of Kabat (Kabat et al., supra).

| 2H7 | Heavy chain (V_{H}) | Light Chain (V_{L}) | Relative |
|---|---|---|---|
| version | substitutions | substitutions | binding |
| 6.8 | (Chimera) | (Chimera) | -1- |
| 2 | (CDR swap) | (CDR swap) | 0.01 |
| 3 | R71V, N73K | (CDR swap) | 0.21 |
| 4 | R71V | (CDR swap) | 0.21 |
| 5 | R71V, N73K | L46P | 0.50 |
| 6 | R71V, N73K | L46P, L47W | 0.58 |
| 7 | R71V | L46P | 0.33 |
| 8 | R71V, L78A | L46P | 0.19 |
| 9 | R71V, F67A | L46P | 0.07 |
| 10 | R71V, F67A, I69L | L46P | 0.12 |
| 11 | R71V, F67A, L78A | L46P | 0.19 |
| 12 | R71V | L46P, M4L | 0.32 |
| 13 | R71V | L46P, M33I | 0.31 |
| 14 | R71V | L46P, F71Y | 0.25 |
| 15 | R71V | L46P, M4L, M33I | 0.26 |
| 16 | R71V, N73K, A49G | L46P | 0.65 |
| 17 | R71V, N73K, A49G | L46P, L47W | 0.67 |

TABLE 5

Oligonucleotide sequences used for construction of
mutations VH(A49G, R71V, N73K) and VL(L46P) in
humanized 2H7 version 16 (2H7.v16). Underlined
codons encode the indicated amino acid
substitutions. For V_{H} (R71V, N73K) and V_{L} (L46P),
the oligos are shown as the sense strand since
these were used for mutagenesis on the Fab
template, while for V_{H} (A49G), the oligo is shown
as the anti-sense strand, since this was used with
the pRK (IgG heavy chain) template. The protein
sequence of version 16 is provided above
under Compositions.

| | |
|---|---|
| Substitution | Oligonucleotide sequence |

| | |
|---|---|
| V_{H} (R71V, N73K) | GT TTC ACT ATA AGT GTC GAC AAG TCC |
| | AAA AAC ACA TT |
| | (SEQ ID NO. 42) |
| V_{H} (A49G) | GCCAGGATAGATGGCGCCAACCCATTCCAGGCC |
| | (SEQ ID NO. 43) |
| V_{L} (L46P) | AAGCTCCGAAACCACTGATTTACGCT |
| | (SEQ ID NO. 44) |

### Example 2

### Additional Mutations within 2H7CDR Regions

Substitutions of additional residues and combinations of substitutions at CDR positions that were identified as important by Ala-scanning were also tested. Several combination variants, particularly v.96 appeared to bind more tightly than v.16.

TABLE 6
Effects of combinations of mutations and non-alanine
substitutions in the CDR regions of humanized 2H7.v16 measured
using cell-based ELISA (WIL2-S cells). The relative binding to
CD20 is expressed as the concentration of the 2H7.v16 parent
over the concentration of the variant required for equivalent binding;
hence a ratio < 1 indicates weaker affinity for the variant; a
ratio > 1 indicates higher affinity for the variant. Standard
deviation in relative affinity determination averaged +/−10%.
Framework substitutions in the variable domains are relative to 2H7.v16
according to the numbering system of Kabat (Kabat et al., supra).

| 2H7 | Heavy chain | Light chain | Relative |
|---|---|---|---|
| Version | substitutions | substitutions | binding |
| 16 | — | — | -1- |
| 96 | D56A, N100A | S92A | 3.5 |
| 97 | S99T, N100G, Y100bI | — | 0.99 |
| 98 | S99G, N100S, Y100bI | — | 1.6 |
| 99 | N100G, Y100bI | — | 0.80 |
| 101 | N54S, D56A | — | 1.7 |
| 102 | N54K, D56A | — | 0.48 |
| 103 | D56A, N100A | — | 2.1 |
| 104 | S99T, N100G | — | 0.81 |
| 105 | S99G, N100S | — | 1.1 |
| 106 | N100G | — | ˜1 |
| 167 | S100aG, Y100bS | — | |
| 136 | D56A, N100A | S56A, S92A | 2.6 |
| 137 | D56A, N100A | A55G, S92A | 2.1 |
| 156 | D56A, N100A | S26A, S56A, S92A | 2.1 |
| 107 | D56A, N100A, Y100bI | S92A | not expressed |
| 182 | Y27W | — | |
| 183 | Y27F | — | |
| 184 | F29Y | — | |
| 185 | F29W | — | |
| 186 | Y32F | — | |
| 187 | Y32W | — | |
| 188 | N33Q | — | |
| 189 | N33D | — | |
| 190 | N33Y | — | |
| 191 | N33S | — | |
| 208 | H35S | — | |
| 209 | A50S | — | |
| 210 | A50R | — | |
| 211 | A50V | — | |
| 212 | A50L | — | |
| 168 | Y52W | — | |
| 169 | Y52F | — | 0.75 |
| 170 | N54D | — | 0.25 |
| 171 | N54S | — | 1.2 |
| 172 | D56K | — | 1 |
| 173 | D56R | — | |
| 174 | D56H | — | 1.5 |
| 175 | D56E | — | 1.2 |
| 213 | D56S | — | |
| 214 | D56G | — | |
| 215 | D56N | — | |
| 216 | D56Y | — | |
| 176 | Y59W | — | |
| 177 | Y59F | — | |
| 180 | K62R | — | |
| 181 | K62D | — | |
| 178 | F63W | — | |
| 179 | F63Y | — | |
| 157 | Y97W | — | 0.64 |
| 158 | Y97F | — | 1.2 |
| 159 | Y98W | — | 0.64 |
| 160 | Y98F | — | 0.88 |
| 106 | N100G | — | |
| 161 | W100cY | — | 0.05 |
| 162 | W100cF | — | 0.27 |
| 163 | F100eY | — | 0.59 |
| 164 | F100eW | — | 0.71 |
| 165 | D101N | — | 0.64 |
| 166 | S99G, N100G, S100aD, | — | 0.99 |
| | Y100b deleted | | |
| 217 | V102Y | — | 1.0 |
| 207 | — | H34Y | |
| 192 | — | Q89E | |
| 193 | — | Q89N | |
| 194 | — | Q90E | |
| 195 | — | Q90N | |
| 196 | — | W91Y | |
| 197 | — | W91F | |
| 205 | — | S92N | |
| 206 | — | S92G | |
| 198 | — | F93Y | |
| 199 | — | F93W | |
| 204 | — | F93S, N94Y | |
| 200 | — | P96L | |
| 201 | — | P96Y | |
| 202 | — | P96W | |
| 203 | — | P96R | |

### Example 3

### Humanized 2H7 Variants with Enhanced Effector Functions

Because 2H7 can mediate lysis of B-cells through both complement-dependent cytotoxicity (CDC) and antibody-dependent cellular cytotoxicity (ADCC), we sought to produce variants of humanized 2H7.v16 with improved CDC and ADCC activity. Mutations of certain residues within the Fc regions of other antibodies have been described (Idusogie et al., J. Immunol. 166:2571-2575 (2001)) for improving CDC through enhanced binding to the complement component C1q. Mutations have also been described (Shields et al., J. Biol. Chem. 276:6591-6604 (2001); Presta et al., Biochem. Soc. Trans. 30:487-490 (2002)) for improving ADCC through enhanced IgG binding to activating Fcγ receptors and reduced IgG binding to inhibitory Fcγ receptors. In particular, three mutations have been identified for improving CDC and ADCC activity: S298A/E333A/K334A (also referred to herein as a triple Ala mutant or variant; numbering in the Fc region is according to the EU numbering system; Kabat et al., supra) as described (Idusogie et al., supra (2001); Shields et al., supra).

In order to enhance CDC and ADCC activity of 2H7, a triple Ala mutant of the 2H7Fc was constructed. A humanized variant of the anti-HER2 antibody 4d5 has been produced with mutations S298A/E333A/K334A and is known as 4D5Fc110 (i.e., anti-p¹⁸⁵HER2 IgG1 (S298A/E333A/K334A); Shields et al., supra). A plasmid, p4D5Fc110 encoding antibody 4D5Fc110 (Shields et al., supra) was digested with ApaI and HindIII, and the Fc-fragment (containing mutations S298A/E333A/K334A) was ligated into the ApaI/HindIII sites of the 2H7 heavy-chain vector pDR2-v 16, to produce pDR2-v31. The amino acid sequence of the version 31 complete H chain is provided above under Compositions. The L chain is the same as that of v16.

Although the constant domains of the Fc region of IgG1 antibodies are relatively conserved within a given species, allelic variations exist (reviewed by Lefranc and Lefranc, in The human IgG subclasses: molecular analysis of structure, function, and regulation, pp. 43-78, F. Shakib (ed.), Pergammon Press, Oxford (1990)).

TABLE 7
Effects of substitutions in the Fc region on CD20 binding.
Relative binding to CD20 was measured in a cell-based (WIL2-S)
assay of framework substitutions. Fc mutations (*) are indicated
by EU numbering (Kabat, supra) and are relative to the 2H7.v16
parent. The combination of three Ala changes in the Fc region of
v.31 is described as “Fc110.” IgG variants are shown with
mutations with respect to the 2H7.v16 background. The relative
binding is expressed as the concentration of the 2H7.v6.8 chimera
over the concentration of the variant required for equivalent binding;
hence a ratio < 1 indicates weaker affinity for the variant. Standard
deviation in relative affinity determination averaged +/−10%.

| 2H7 | Fc | Relative |
|---|---|---|
| version | Substitutions* | binding |
| 6.8 | — | -1- |
| 16 | — | 0.65 |
| 31 | S298A, E333A, K334A | 0.62 |

### Example 4

### Humanized 2H7 Variants with Enhanced Stability

For development as therapeutic proteins, it is desirable to choose variants that remain stable with respect to oxidation, deamidation, or other processes that may affect product quality, in a suitable formulation buffer. In 2H7.v16, several residues were identified as possible sources of instability: VL (M32) and VH (M34, N100). Therefore, mutations were introduced at these sites for comparison with v16.

TABLE 8
Relative binding of 2H7 variants designed for enhanced stability
and/or effector function, to CD20 in a cell-based (WIL2-S) assay.
IgG variants are shown with mutations with respect to the 2H7.v16
background. The relative binding is expressed as the concentration
of the 2H7.v6.8 chimera over the concentration of the variant required
for equivalent binding; hence a ratio < 1 indicates weaker affinity for
the variant. Standard deviation in relative affinity determination
averaged +/−10%. Framework substitutions in the variable
domains are relative to 2H7.v16 according to the numbering system
of Kabat and Fc mutations (*) are indicated by EU numbering (Kabat
et al., supra). (**) Variants that were measured with 2H7.v16 as the
standard comparator; relative values are normalized to that of the chimera.
Additional Fc mutations were combined with stability or affinity-
enhancing mutations to alter or enhance effector functions based on
previously reported mutations (Idusogie et al. (2000); Idusogie et al.
(2001); Shields et al. (2001)). These changes include S298, E333A,
K334A as described above; K322A to reduced CDC activity; D265A
to reduce ADCC activity; K326A or K326W to enhance CDC
activity; and E356D/M358L to test the effects of allotypic changes
in the Fc region. None of these mutations caused significant
differences in CD20 binding affinity.

| 2H7 | | | | |
|---|---|---|---|---|
| Ver- | Heavy chain | Light chain | | Relative |
| sion | (V_{H}) changes | (V_{L}) changes | Fc changes* | binding |
| 6.8 | (chimera) | (chimera) | — | -1- |
| 16 | — | — | — | 0.65 |
| 62 | — | M32I | — | 0.46 |
| 63 | M34I | — | — | 0.49 |
| 64 | N100A | — | — | |
| 65 | N100A | L47W | — | 0.74 |
| 66 | S99A | L47W | — | 0.62 |
| 67 | N54A | — | — | |
| 68 | — | M32I | — | 0.48 |
| 69 | — | M32L | — | 0.52 |
| 70 | N100A | — | S298A, E333A, K334A | 0.80 |
| 71 | N100D | — | 5298A, E333A, K334A | 0.44 |
| 72 | N100A | M32I | — | 0.58 |
| 73 | N100A | M32L | — | 0.53 |
| 74 | N100A | M32I | S298A, E333A, K334A | 0.61 |
| 75 | N100A | M32L | S298A, E333A, K334A | 0.60 |
| 113 | — | — | E356D, M358L | 0.60** |
| 114 | D56A, N100A | M32L, S92A | S298A, E333A, K334A | 1.2** |
| 115 | D56A, N100A | M32L, S92A | S298A, E333A, K334A, | 1.4** |
| | | | E356D, M358L | |
| 116 | D56A, N100A | M32L, S92A | S298A, K334A, K322A | 1.2** |
| 134 | D56A, N100A | M32L, S92A | E356D, M358L, D265A | 1.5** |
| 135 | D56A, N100A | M32L, S92A | E356D, M358L, D265A, | 0.95** |
| | | | K326W | |
| 138 | D56A, N100A | M32L, S92A | S298A, E333A, K334A, | 1.2** |
| | | | K326A | |
| 139 | D56A, N100A | M32L, S92A | S298A, E333A, K334A, | 1.1** |
| | | | K326A, E356N, M358L | |
| 154 | — | — | D265A | 0.70** |
| 155 | — | — | S298A, K322A, K334A | 0.70** |

**Variants that were measured with 2H7.v16 as comparator;
relative binding values are normalized to that of the chimera.

To test the effects of stability mutations on the rate of protein degradation, 2H7.v16 and 2H7.v73 were formulated at 12-14 mg/mL in 10 mM histidine, 6% sucrose, 0.02% polysorbate 20, pH 5.8 and incubated at 40° C. for 16 days. The incubated samples were then assayed for changes in charge variants by ion exchange chromatography, aggregation and fragmentation by size exclusion chromatography, and relative binding by testing in a cell-based (WIL2-S) assay.

The results showed that 2H7 v.73 has greater stability compared to 2H7 v.16 with respect to losses in the fraction of main peak by ion exchange chromatography under accelerated stability conditions. No significant differences were seen with respect to aggregation, fragmentation, or binding affinity.

### Example 5

### Complement Dependent Cytotoxicity (CDC) Assays

2H7 IgG variants were assayed for their ability to mediate complement-dependent lysis of WIL2-S cells, a CD20 expressing lymphoblastoid B-cell line, essentially as described (Idusogie et al., J. Immunol. 164:4178-4184 (2000); Idusogie et al., J. Immunol. 166:2571-2575 (2001)). Antibodies were serially diluted 1:3 from a 0.1 mg/mL stock solution. A 0.05 mL aliquot of each dilution was added to a 96-well tissue culture plate that contained 0.05 mL of a solution of normal human complement (Quidel, San Diego, Calif.) To this mixture, 50,000 WIL2-S cells were added in a 0.05 mL volume. After incubation for 2 h at 37° C., 0.05 mL of a solution of Alamar blue (Accumed International, Westlake, Ohio) was added, and incubation was continued for an additional 18 h at 37° C. Covers were then removed from the plates, and they were shaken for 15 min at room temperature on an orbital shaker. Relative fluorescent units (RFU) were read using a 530 nm excitation filter and a 590 nm emission filter. An EC₅₀ was calculated by fitting RFU as a function of concentration for each antibody using KaleidaGraph software.

The results (Table 9) show surprising improvement in CDC by humanized 2H7 antibodies, with relative potency similar to Rituxan® for v.73, 3-fold more potent than Rituxan® for v.75, and 3-fold weaker than Rituxan® for v.16.

TABLE 9
CDC activity of 2H7 antibodies compared to Rituxan. Numbers > 1
indicate less potent CDC activity than Rituxan ®
and numbers < 1 indicate more potent activity than
Rituxan ®. Antibodies were produced from stable CHO lines,
except that those indicated by (*) were produced transiently.

| Antibody variant | n | EC₅₀(variant)/EC₅₀(Rituxan) |
|---|---|---|
| Rituxan ® | 4 | -1- |
| 2H7.v16 | 4 | 3.72; 4.08 |
| 2H7.v31* | 4 | 2.21 |
| 2H7.v73 | 4 | 1.05 |
| 2H7.v75 | 4 | 0.33 |
| 2H7.v96* | 4 | 0.956 |
| 2H7.v114* | 4 | 0.378 |
| 2H7.v115* | 4 | 0.475 |
| 2H7.v116* | 1 | >100 |
| 2H7.v135* | 2 | 0.42 |

### Example 6

### Antibody Dependent Cellular Cytotoxicity (ADCC) Assays

2H7 IgG variants were assayed for their ability to mediate Natural-Killer cell (NK cell) lysis of WIL2-S cells, a CD20 expressing lymphoblastoid B-cell line, essentially as described (Shields et al., J. Biol. Chem. 276:6591-6604 (2001)) using a lactate dehydrogenase (LDH) readout. NK cells were prepared from 100 mL of heparinized blood, diluted with 100 mL of PBS (phosphate buffered saline), obtained from normal human donors who had been isotyped for FcγRIII, also known as CD16 (Koene et al., Blood 90:1109-1114(1997)). In this experiment, the NK cells were from human donors heterozygous for CD16 (F1581V158). The diluted blood was layered over 15 mL of lymphocyte separation medium (ICN Biochemical, Aurora, Ohio) and centrifuged for 20 min at 2000 RPM. White cells at the interface between layers were dispensed to 4 clean 50-mL tubes, which were filled with RPMI medium containing 15% fetal calf serum. Tubes were centrifuged for 5 min at 1400 RPM and the supernatant discarded. Pellets were resuspended in MACS buffer (0.5% BSA, 2 mM EDTA), and NK cells were purified using beads (NK Cell Isolation Kit, 130-046-502) according to the manufacturer's protocol (Miltenyi Biotech,). NK cells were diluted in MACS buffer to 2×10⁶ cells/mL.

Serial dilutions of antibody (0.05 mL) in assay medium (F12/DMEM 50:50 without glycine, 1 mM HEPES buffer pH 7.2, Pennicillin/Streptomycin (100 units/mL; Gibco), glutamine, and 1% heat-inactivated fetal bovine serum) were added to a 96-well round-bottom tissue culture plate. WIL2-S cells were diluted in assay buffer to a concentration of 4×10⁵/mL. WIL2-S cells (0.05 mL per well) were mixed with diluted antibody in the 96-well plate and incubated for 30 min at room temperature to allow binding of antibody to CD20 (opsonization).

The ADCC reaction was initiated by adding 0.1 mL of NK cells to each well. In control wells, 2% Triton X-100 was added. The plate was then incubated for 4 h at 37° C. Levels of LDH released were measured using a cytotoxicity (LDH) detection kit (Kit#1644793, Roche Diagnostics, Indianapolis, Ind.) following the manufacturers instructions. 0.1 mL of LDH developer was added to each well, followed by mixing for 10s. The plate was then covered with aluminum foil and incubated in the dark at room temperature for 15 min. Optical density at 490 nm was then read and use to calculate % lysis by dividing by the total LDH measured in control wells. Lysis was plotted as a function of antibody concentration, and a 4-parameter curve fit (KaleidaGraph) was used to determine EC₅₀ concentrations.

The results showed that humanized 2H7 antibodies were active in ADCC, with relative potency 20-fold higher than Rituxan® for v.31 and v.75, 5-fold more potent than Rituxan® for v.16, and almost 4-fold higher than Rituxan® for v.73.

TABLE 10
ADCC activity of 2H7 antibodies on WIL2-S cells compared to
2H7.v16, based on n experiments. (Values > 1 indicate lower
potency than 2H7.v16, and values < 1 indicate greater potency.)

| Antibody variant | n | EC₅₀(variant)/EC₅₀(2H7.v16) |
|---|---|---|
| Rituxan ® | 4 | 5.3 |
| 2H7.v16 | 5 | 1 |
| 2H7.v31 | 1 | 0.24 |
| 2H7.v73 | 5 | 1.4 |
| 2H7.v75 | 4 | 0.25 |

Additional ADCC assays were carried out to compare combination-variants of 2H7 with Rituxan®. The results of these assays indicated that 2H7.v114 and 2H7.v115 have >10-fold improved ADCC potency as compared to Rituxan® (Table 11).

TABLE 11
ADCC activity of 2H7 antibodies on WIL2-S cells compared
to Rituxan ®, based on n experiments (Values > 1
indicate lower potency than Rituxan ®, and
values < 1 indicate greater potency).

| Antibody | | |
|---|---|---|
| variant | | EC50(variant)/EC50(Rituxan) |
| Rituxan ® | 2 | -1- |
| 2H7 v.16 | 2 | 0.52 |
| 2H7 v.96 | 2 | 0.58 |
| 2H7.v114 | 2 | 0.093 |
| 2H7.v115 | 2 | 0.083 |
| 2H7.v116 | 2 | 0.30 |

### Example 7

### Assays for FcRn Binding

Soluble Fc variants with amino acid alterations in the Fc (e.g., N434W, N434Y, N434F, N434A N434H, N434A+E380A+T307A) were expressed, purified, and assayed in a BIAcore binding assay for their affinity for human, cynomolgus monkey, rat, and murine FcRn. The Fc variants were also analyzed by size exclusion chromatography to determine their aggregation tendencies.

Variant Fc fragments were expressed by transforming 34B8 E. coli cells with the mutant pW0437 phagemids, growing for 24 hours at 30° C. in phosphate-free media to induce expression of the Fc genes, and harvesting the cells. Cell paste was frozen overnight, and lysed by osmotic shock in 10 mM Tris, 1 mM EDTA. Lysate was cleared by centrifugation and applied to a Protein A column. The column was washed with PBS, soluble Fc eluted with Protein A Citrate Elution Buffer (0.1 M citrate, pH 3.0), and neutralized with Tris pH 7.5. Soluble Fc was concentrated in an Amicon Centriprep.

FcRn from human, cynomolgus monkey, rat, or mouse was immobilized by NHS chemistry on Biacore CM5 chips at varying densities (100-3000 RU). Fc variants were serially diluted from 10 μM to 1 nM in PBS at pH 6.0, and binding was monitored over time. For the parental hingeless Fc (i.e., wild-type), equilibrium binding was reached almost immediately for huFcRn, indicating that it has very fast on- and off-rates, and an approximate Kd of 700 nM as determined by equilibrium analysis. For the N434W variant, the on-rate is noticeably slower, and the off-rate is extremely slow. By injecting N434W at a slower flow rate and for a longer period of time, equilibrium analysis was possible, and the Kd is approximately 4 nM. The variants N434W, N434F, N434A, and the triple mutant N434A+E380A+T307A had apparent increases in affinity over wild-type Fc of ˜170-fold, ˜9-fold, ˜2.7-fold, and ˜14-fold, respectively, at pH 6.0. In contrast, at pH 7.4, affinity of the N434 variants for huFcRn is effectively too low to be measured in this assay. The improvement of N434W relative to wild-type is not as significant for binding to cyno FcRn, showing only about 10-fold better for binding to rat FcRn, and virtually the same as WT for binding to murine FcRn. Thus the improvement achieved by this mutation is a specific for human FcRn.

Human IgG1 variants of 2H7.v138 were analyzed for pH-dependent binding to human FcRn in an ELISA using biotinylated FcRn. MaxiSorp 96-well microwell plates (Nunc, Roskilde, Denmark) were coated with 2 μg/ml NeutrAvidin (Pierce, Rockford, Ill.), at 100 μl/well in 50 mM carbonate buffer, pH 9.6, at 4° C. overnight. Plates were washed with PBS containing 0.05% polysorbate (wash buffer), pH 7.4, and blocked with PBS containing 0.5% BSA, pH 7.4, at 150 μm/well. After a one-hour incubation at room temperature, plates were washed with wash buffer, pH 7.4. Human FcRn was biotinylated using biotin-X-NHS (Research Organics, Cleveland, Ohio). Biotinylated FcRn was added to the plates at 2 μg/ml, 100 μl/well, in PBS containing 0.5% BSA, 0.05% polysorbate 20 (sample buffer), pH 7.4. The plates were incubated for one hour and washed with wash buffer, pH 6.0. Seven twofold serial dilutions of IgG antibodies (3.1-200 ng/ml) in sample buffer, pH 6.0, were added to the plates. After a two-hour incubation, plates were washed with wash buffer, pH 6.0. Bound IgG was detected by adding peroxidase labeled goat F(ab′)₂ anti-human IgG F(ab′)₂ (Jackson ImmunoResearch, West Grove, Pa.) at 100 μl/well in sample buffer, pH 6.0. After a one-hour incubation, plates were washed with wash buffer, pH 6.0, and the substrate 3,3′,5,5′-tetramethyl benzidine (TMB) (Kirkegaard & Perry Laboratories) was added at 100 μl/well. The reaction was stopped by adding 1 M phosphoric acid at 100 μl/well. Absorbance was read at 450 nm on a multiskan Ascent reader (Thermo Labsystems, Helsinki, Finland). The absorbance at the midpoint of the standard curve (mid-OD) was calculated. The corresponding concentrations of standard and samples at this mid-OD were determined from the titration curves using a four-parameter nonlinear regression curve-fitting program (KaleidaGraph, Synergy software, Reading, Pa.). The relative activity was calculated by dividing the mid-OD concentration of standard by that of sample.

For evaluation of dissociation of bound IgG from FcRn at pH 6.0 or pH 7.4, the assay was carried out similarly except that after the sample incubation step and when the plates were washed, sample buffer at pH 6.0 or 7.4 was added at 100 μl/well. Plates were incubated for 45 min and washed. The assay was then continued as described above.

### Example 8

### Characterization of Humanized Anti-CD20 IgG1 Variants with FcRn Mutations

Mutations identified by phage display of human Fc were also tested for their effects in the background of an intact antibody, 2H7.v138. 2H7.v138 is a humanized anti-CD20 antibody in which the Fc has been modified for increased ADCC and CDC activities through the following mutations: S298A, K326A, E333A, K334A. Mutations at position N434 were introduced into this background, and IgG (Table 12) prepared by transient transfection of 293 cells as previously described. In each case, purified IgG variants were shown to have low levels of protein aggregation by size-exclusion chromatography as described above.

TABLE 12
Variants of humanized anti-CD20 antibody 2H7.v138

| 2H7 variant | FcRn mutation |
|---|---|
| 138 | — |
| 364 | N434A |
| 477 | N434W |
| 478 | N434F |
| 479 | N434Y |

Human IgG1 variants of 2H7.v138 were analyzed for pH-dependent binding to human FcRn in an ELISA using biotinylated FcRn. MaxiSorp 96-well microwell plates (Nunc, Roskilde, Denmark) were coated with 21 g/ml NeutrAvidin (Pierce, Rockford, Ill.), at 100 μl/well in 50 mM carbonate buffer, pH 9.6, at 4° C. overnight. Plates were washed with PBS containing 0.05% polysorbate (wash buffer), pH 7.4, and blocked with PBS containing 0.5% BSA, pH 7.4, at 150 μl/well. After a one-hour incubation at room temperature, plates were washed with wash buffer, pH 7.4. Human FcRn was biotinylated using biotin-X-NHS (Research Organics, Cleveland, Ohio). Biotinylated FcRn was added to the plates at 2 μg/ml, 100 μl/well, in PBS containing 0.5% BSA, 0.05% polysorbate 20 (sample buffer), pH 7.4. The plates were incubated for one hour and washed with wash buffer, pH 6.0. Seven twofold serial dilutions of IgG antibodies (3.1-200 ng/ml) in sample buffer, pH 6.0, were added to the plates. After a two-hour incubation, plates were washed with wash buffer, pH 6.0. Bound IgG was detected by adding peroxidase labeled goat F(ab′)₂ anti-human IgG F(ab′)₂ (Jackson ImmunoResearch, West Grove, Pa.) at 100 μl/well in sample buffer, pH 6.0. After a one-hour incubation, plates were washed with wash buffer, pH 6.0, and the substrate 3,3′,5,5′-tetramethyl benzidine (TMB) (Kirkegaard & Perry Laboratories) was added at 100 μl/well. The reaction was stopped by adding 1 M phosphoric acid at 100 μl/well. Absorbance was read at 450 nm on a multiskan Ascent reader (Thermo Labsystems, Helsinki, Finland). The absorbance at the midpoint of the standard curve (mid-OD) was calculated. The corresponding concentrations of standard and samples at this mid-OD were determined from the titration curves using a four-parameter nonlinear regression curve-fitting program (KaleidaGraph, Synergy software, Reading, Pa.). The relative activity was calculated by dividing the mid-OD concentration of standard by that of sample.

For evaluation of dissociation of bound IgG from FcRn at pH 6.0 or pH 7.4, the assay was carried out similarly except that after the sample incubation step and when the plates were washed, sample buffer at pH 6.0 or 7.4 was added at 100 μl/well. Plates were incubated for 45 min and washed. The assay was then continued as described above.

The results indicated relative binding affinities similar to those observed for the Fc variants. At pH 6.0, the relative binding affinities are v477>v478=v479>v364>v138. At pH 7.4, the relative binding affinities are consistently weaker than at pH 6.0, with the same relative binding: v477>v478=v479>v364>v138.

These Fc mutations are broadly applicable to human IgG antibodies.

### Example 9

### In vivo Effects of 2H7 Variants in a Pilot Study in Cynomolgus Monkeys

2H7 variants, produced by transient transfection of CHO cells, were tested in normal male cynomolgus (Macaca fascicularis) monkeys in order to evaluate their in vivo activities. Other anti-CD20 antibodies, such as C2B8 (Rituxan®) have demonstrated an ability to deplete B-cells in normal primates (Reff et al., Blood 83: 435-445 (1994)).

In one study, humanized 2H7 variants were compared. In a parallel study, Rituxan® was also tested in cynomolgus monkeys. Four monkeys were used in each of five dose groups: (1) vehicle, (2) 0.05 mg/kg hu2H7.v16, (3) 10 mg/kg hu2H7.v16, (4) 0.05 mg/kg hu2H7.v31, and (5) 10 mg/kg hu2H7.v31. Antibodies were administered intravenously at a concentration of 0, 0.2, or 20 mg/mL, for a total of two doses, one on day 1 of the study, and another on day 8. The first day of dosing is designated day 1 and the previous day is designated day-1; the first day of recovery (for 2 animals in each group) is designated as day 11. Blood samples were collected on days-19, -12, 1 (prior to dosing), and at 6 h, 24 h, and 72 h following the first dose. Additional samples were taken on day 8 (prior to dosing), day 10 (prior to sacrifice of 2 animals/group), and on days 36 and 67 (for recovery animals).

Peripheral B-cell concentrations were determined by a FACS method that counted CD3−/CD40+ cells. The percent of CD3-CD40+B cells of total lymphocytes in monkey samples were obtained by the following gating strategy. The lymphocyte population was marked on the forward scatter/side scatter scattergram to define Region 1 (R1). Using events in R1, fluorescence intensity dot plots were displayed for CD40 and CD3 markers. Fluorescently labeled isotype controls were used to determine respective cutoff points for CD40 and CD3 positivity.

The results indicated that both 2H7.v16 and 2H7.v31 were capable of producing full peripheral B-cell depletion at the 10 mg/kg dose and partial peripheral B-cell depletion at the 0.05 mg/kg dose. The time course and extent of B-cell depletion measured during the first 72 h of dosing were similar for the two antibodies. Subsequent analysis of the recovery animals indicated that animals treated with 2H7.v31 showed a prolonged depletion of B-cells as compared to those dosed with 2H7.v16. In particular, recovery animals treated with 10 mg/kg 2H7.v16, B-cells showed substantial B-cell recovery at some time between sampling on Day 10 and on Day 36. However, for recovery animals treated with 10 mg/kg 2H7.v31, B-cells did not show recovery until some time between Day 36 and Day 67. This suggests a greater duration of full depletion by about one month for 2H7.v31 compared to 2H7.v16.

No toxicity was observed in the monkey study at low or high dose and the gross pathology was normal. In other studies, v16 was well tolerated up to the highest dose evaluated of (100 mg/kg×2=1200 mg/m²×2) following i.v. administration of 2 doses given 2 weeks apart in these monkeys.

Data in Cynomolgus monkeys with 2H7.v16 versus Rituxan® suggests that a 5-fold reduction in CDC activity does not adversely affect potency. An antibody with potent ADCC activity but reduced CDC activity may have more favorable safety profile with regard to first infusion reactions than one with greater CDC activity.

### Example 10

### In Vivo Suppression of Tumor Growth

The ability of rhuMAb 2H7.v 16 to inhibit the growth of the Raji human B-cells, a lymphoma cell line (ATCC CCL 86), was evaluated in Balb/c nude (athymic) mice. The Raji cells express CD20 and have been reported to grow in nude mice, producing metastatic disease; tumor growth is inhibited by Rituxan® (Clynes et al., Nature Medicine 6, 443-446 (2000)). Fifty-six 8-10 week old, Balb/c nude mice were divided into 7 groups (A-G) with each group consisting of 8 mice. On day 0, each mouse received a subcutaneous injection of 5×10⁶ Raji B-lymphoma cells in the flank. Beginning at day 0, each mouse received either 100 uL of the negative-control solution (PBS; phosphate-buffered saline), Rituxan® or 2H7.v16. Dosage was dependent on weight and drug delivery was intravenously via the tail vein. Group A mice received PBS. Groups B-D received Rituxan® at 5.0, mg/kg, 0.5 mg/kg, and 0.05 mg/kg respectively. Groups E-G mice received 2H7 v.16 at 5.0 mg/kg, 0.5 mg/kg, and 0.05 mg/kg respectively. The injections were repeated every week for 6 weeks. At weekly intervals during treatment, each mouse was inspected for the presence of palpable tumors at the site of injection, and the volume of the tumors if present were measured and recorded. A final inspection was made at week 8 (after a two-week interval of no treatments).

The results of this study showed that both rhuMAb 2H7.v 16 and Rituxan® and were effective at inhibiting subcutaneous Raji-cell tumor growth in nude mice. Tumor growth was observed in the PBS control group beginning at 4 weeks. However, no tumor growth was observed in groups treated with Rituxan® or 2H7.v16 at 5 mg/kg or 0.5 mg/kg for the 8-week duration of the study. In the low-dose 0.05 mg/kg treatment groups, tumors were observed in one animal in the 2H7 group and in one animal in the Rituxan® group.

### Example 11

### Phase I/II Study of rhuMAb 2H7 (2H7.v16) in Moderate to Severe Rheumatoid Arthritis Objectives

The primary objective of this study was to evaluate the safety and tolerability of escalating intravenous (IV) doses of rhuMAb 2H7 in subjects with moderate to sever rheumatoid arthritis (RA).

Study Design

This was a randomized, placebo-controlled, multicenter, blinded Phase I/II, investigator- and subject-blinded study of the safety of escalating doses of rhuMAb 2H7 (PRO70769) in combination with MTX in subjects with moderate to severe RA. The study consisted of a dose escalation phase and a second phase with enrollment of a larger number of subjects. The Sponsor will remain unblinded to treatment assignment.

Subjects with moderate to severe RA who have failed one to five disease-modifying antirheumatic drugs or biologics who currently have unsatisfactory clinical responses to treatment with MTX were enrolled.

Subjects were required to receive MTX in the range of 10-25 mg weekly for at least 12 weeks prior to study entry and to be on a stable dose for at least 4 weeks before receiving their initial dose of study drug (PRO70769 or placebo). Subjects may also receive stable doses of oral corticosteroids (up to 10 mg daily or prednisone equivalent) and stable doses of nonsteroidal anti-inflammatory drugs (NSAIDs). Subjects received two IV infusions of PRO70769 or placebo equivalent at the indicated dose on Days 1 and 15 according to the following dose escalation plan.

Dose escalation occurred according to specific criteria and after review of safety data by an internal safety data review committee and assessment of acute toxicity 72 hours following the second infusion in the last subject treated in each cohort. After the dose escalation phase, 40 additional subjects (32 active and 8 placebo) will be randomized to each of the following dose levels: 2×50 mg, 2×200 mg, 2×500 mg, and 2×1000 mg, if the dose levels have been demonstrated to be tolerable during the dose escalation phase. Approximately 205 subjects will be enrolled in the study.

B-cell counts will be obtained and recorded. B-cell counts will be evaluated using flow cytometry in a 48-week follow-up period beyond the 6-month efficacy evaluation. B-cell depletion will not be considered a dose-limiting toxicity (DLC), but rather the expected pharmacodynamic outcome of PRO70769 treatment.

In a substudy, blood for serum and RNA analyses, as well as urine samples will be obtained from subjects at various timepoints. These samples may be used to identify biomarkers that may be predictive of response to PRO70769 treatment in subjects with moderate to severe RA.

Study Treatment

Cohorts of subjects received two IV infusions of PRO70769 or placebo equivalent at the indicated dose on Days 1 and 15 according to the following escalation plan:

10 mg PRO70769 or placebo equivalent: 4 subjects active drug, 1 control

50 mg PRO70769 or placebo equivalent: 8 subjects active drug, 2 control

200 mg PRO70769 or placebo equivalent: 8 subjects active drug, 2 control

500 mg PRO70769 or placebo equivalent: 8 subjects active drug, 2 control

1000 mg PRO70769 or placebo equivalent: 8 subjects active drug, 2 control

Efficacy

The efficacy of PRO70769 will be measured by ACR responses. The percentage of subjects who achieve an ACR20, ACR50, and ACR70 response will be summarized by treatment group and 95% confidence intervals will be generated for each group. The components of these response and their change from baseline will be summarized by treatment and visit.

### Example 12

From inspection of the crystal structure of 2H7.v 16 combined with alanine scanning data, it appeared that the region of CDR-H3 including N100 (V_{H}) and neighboring residues might be involved in antigen binding. We therefore focused on N100 (V_{H}), making a number of amino acid substitutions in the 2H7.v16 background. The results (Table 13) indicated that either Tyr or Trp substitutions improved binding relative to v16.

TABLE 13
Relative binding of 2H7 variants designed for enhanced binding
affinity to CD20, as measured in a cell-based (WIL2-S) assay with IgG
variants produced in transfected 293 cells. IgG variants are indicated
with mutations (Kabat numbering) with respect to the 2H7.v16
background. Relative binding is expressed as the concentration of
2H7.v16 over the concentration of the variant required for equivalent
binding; hence, a ratio > 1 indicates improved binding affinity of the
variant for CD20 compared to version 16.

| 2H7 | Heavy Chain | Relative |
|---|---|---|
| version | (V_{H}) changes | binding |
| 16 | — | -1- |
| 460 | N100R | 1.44 |
| 461 | N100Q | 1.15 |
| 462 | N100E | 0.8 |
| 463 | N100G | 0.76 |
| 464 | N100H | 1.17 |
| 465 | N100I | 0.86 |
| 466 | N100L | 0.81 |
| 467 | N100K | 0.74 |
| 468 | N100F | 1.21 |
| 469 | N100P | 0.9 |
| 470 | N100S | 1.08 |
| 471 | N100T | 1 |
| 472 | N100Y | 1.53 |
| 473 | N100W | 1.91 |
| 474 | N100V | 0.79 |
| 475 | N100M | 0.75 |

To determine whether additional affinity improvements could be obtained by substitutions at S100a (VH), a number of mutations at this position were made, in this case in the background of 2H7.v472 (Table 13). The results of cell-based binding comparisons (Table 14) indicated that 2H7.v511 had >3 fold improved binding to CD20 on cells.

The gene for the 2H7.v511 heavy chain was constructed by site-directed mutagenesis using ssDNA of a plasmid encoding the heavy chain of 2H7.v 138 as template and a 5′-phosphorylated deoxyoligonucleotide designated as CA1568: 5′-CCA GAC GTC GAA GTA CCA GTA GCG GTA GCT ATAGTA TAC GAC GCG-3′ (SEQ ID NO. 45). The underlined nucleotides correspond to codons of the antisense DNA strand, encoding the CDR-H3 changes N100Y, S100aR. The light chain of 2H7.v511 is identical to that of 2H7.v138, and the same plasmid was used for expression of the light chain.

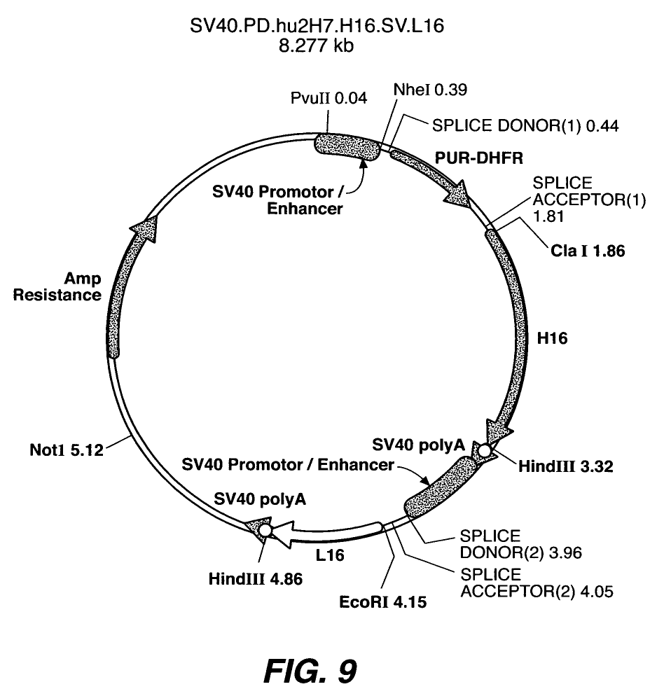

Hu2H7 variants such as v138 and v511 can be expressed in CHO cells using the vector shown in FIG. 9 used for expression of 2H7.v16.

TABLE 14
Relative binding of 2H7 variants designed for enhanced binding
affinity to CD20, as measured in a cell-based (WIL2-S) assay
with IgG variants produced in transfected 293 cells. IgG variants
are indicated with mutations (Kabat numbering) at CDR sites
as compared to the 2H7.v16 background. In addition, all variants
contain Fc changes S298A, K326A, E333A, and K334A (EU
numbering), as compared to 2H7.v16. Relative binding is
expressed as the concentration of 2H7.v16 over the concentration
of the variant required for equivalent binding; hence, a ratio > 1
indicates improved binding affinity of the variant for CD20
compared to version 16.

| 2H7 | VL | VL | VH | VH | VH | Relative |
|---|---|---|---|---|---|---|
| version | 32 | 92 | 56 | 100 | 100a | Binding |
| 16 | M | S | D | N | S | -1- |
| 590 | L | A | A | W | R | |
| 511 | L | A | A | Y | R | 3.55 |
| 512 | L | A | A | Y | N | 2.12 |
| 513 | L | A | A | Y | D | 1.65 |
| 515 | L | A | A | Y | E | 1.92 |
| 516 | L | A | A | Y | G | 2.34 |
| 517 | L | A | A | Y | H | 2.2 |
| 518 | L | A | A | Y | I | 2.0 |
| 519 | L | A | A | Y | L | 1.7 |
| 520 | L | A | A | Y | K | 1.55 |
| 521 | L | A | A | Y | F | 2.21 |
| 522 | L | A | A | Y | P | 1.57 |
| 523 | L | A | A | Y | T | 2.41 |
| 524 | L | A | A | Y | V | 1.54 |
| 525 | L | A | A | Y | M | 2.31 |

Because these variants also included Fc changes, we additionally measured binding in a competitive Scatchard assay to determine the apparent equilibrium dissociation constant (K_{d}).

Equilibrium dissociation constants (K_{d}) were determined for humanized anti-CD20 monoclonal antibodies binding to WIL2s-S cells using radiolabeled antibodies 2H7.v511 and 2H7.v138 IgG. Anti-CD20 monoclonal antibodies were produced in CHO cells. All dilutions were performed in binding assay buffer (DMEM media containing 0.5% bovine serum albumin, 25 mM HEPES pH 7.2, and 0.01% sodium azide). Aliquots (0.05 mL) of ¹²⁵I-2H7.v511, and ¹²⁵I-2H7.v138 (iodinated with lactoperoxidase) at a concentration of 0.1 nM were dispensed into wells of a V-bottom 96-well microassay plate. Serial dilutions (0.05 mL) of unlabelled antibody were added and mixed. 30,000 WIL2-S cells in a volume of 0.05 mL was added to each well. The plates were sealed and incubated at room temperature for 24 h, then centrifuged for 15 min at 2,500 RPM. The supernatant was then aspirated and the cell pellets were washed and centrifuged. The supernatant was again aspirated, and the pellets were dissolved in 1N NaOH and transferred to tubes for gamma counting. The data were used for Scatchard analysis (Munson and Rodbard, Anal. Biochem. 107:220-239 (1980)) using the program Ligand (McPherson, Comput. Programs Biomed. 17: 107-114 (1983)). The results, shown in Table 15 below, indicate that anti-CD20 monoclonal antibodies 2H7.v511 and 2H7.v138 bind with high affinity to CD20 expressed on WIL2-S cells and that 2H7.v511 binds with 2.2-fold to 7.3-fold higher affinity than 2H7.v116. The difference in values obtained with labeled ¹²⁵I-2H7.v511, and ¹²⁵I-2H7.v 138 may reflect perturbation of antigen binding resulting from iodination of the new Tyr in CDR-H3 of 2H7.v511. It therefore seems likely that the ¹²⁵I-2H7.v138 results more accurately reflect the relative binding affinities.

TABLE 15
Equilibrium binding affinity of anti-CD20 humanized monoclonal
antibodies from Scatchard analysis (+/− standard error of fit).

| Competitor | Labeled | Apparent | Relative |
|---|---|---|---|
| Antibody | Antibody | K_{d} (nM) | affinity |
| 2H7.v16 | ¹²⁵I-2H7.v138 | 0.80 ± 0.07 | -1- |
| 2H7.v511 | ¹²⁵I-2H7.v138 | 0.11 ± 0.05 | 7.3 |
| 2H7.v16 | ¹²⁵I-2H7.v511 | 0.98 ± 0.12 | -1- |
| 2H7.v511 | ¹²⁵I-2H7.v511 | 0.45 ± 0.05 | 2.2 |

As an additional comparison of the binding activity of 2H7 variants, an ELISA was performed with solubilized CD20. For comparison, another variant, 2H7.v588, was constructed with CDR regions identical to 2H7.v16 and Fc regions identical to 2H7.v511.

NUNC Maxisorp™ plates were coated with 2.5 μg/ml of soluble CD20 (Genentech; prepared as previously described) in PBS, overnight at 4° C., and then blocked with 0.5% BSA at room temperature for 1 h. Serial dilutions of samples in 0.5% BSA were incubated on the plates for 2 h. After washing, bound antibodies were detected with HRP-conjugated anti-human Fab antibody (Goat anti-human Ig, Fab′2-HRP conjugate, from NB) using 3,3′,5,5′-tetramethyl benzidine (Kirkegaard and Perry Laboratories, Gaithersburg, Md.) as substrate. Absorbance was read at 450 nm. Titration curves were fit with a four-parameter nonlinear regression curve-fitting program (KaleidaGraph; Synergy Software, Reading, Pa.). Concentrations of antibody variants corresponding to the midpoint absorbance of the titration curve of the standard were calculated. The results (FIG. 3) showed similar binding affinity for 2H7.v588 and 2H7.v16; however, 2H7.v511 appeared to have approximately 90-fold enhanced binding to soluble CD20 in this assay.

### Example 13

### ADCC Activity

ADCC activity was evaluated as previously described using WIL2-S cells and purified NK cells from normal human donors with an effector:target ratio of about 4 for FIG. 4 and ratio of 5:1 for the FIG. 11 assay. The results, shown in FIG. 4 and FIG. 11 indicated increased ADCC potency and maximal activity for 2H7.v511 as compared to 2H7.v16 and other 2H7 variants. 2H7.v588 showed similar potency and maximal activity to 2H7.v511 (FIG. 4), indicating that further CD20 affinity improvements may not significantly enhance ADCC activity.

### Example 14

### CDC Activity

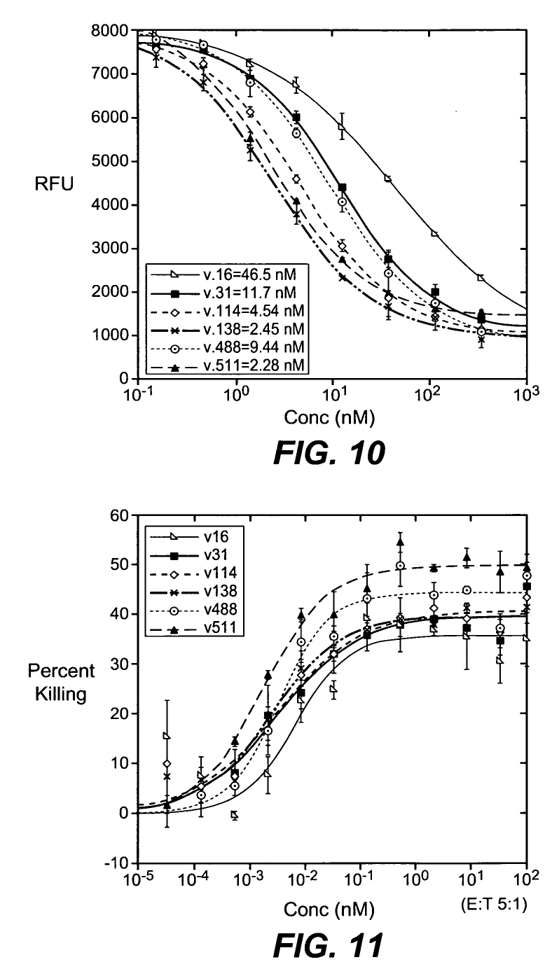

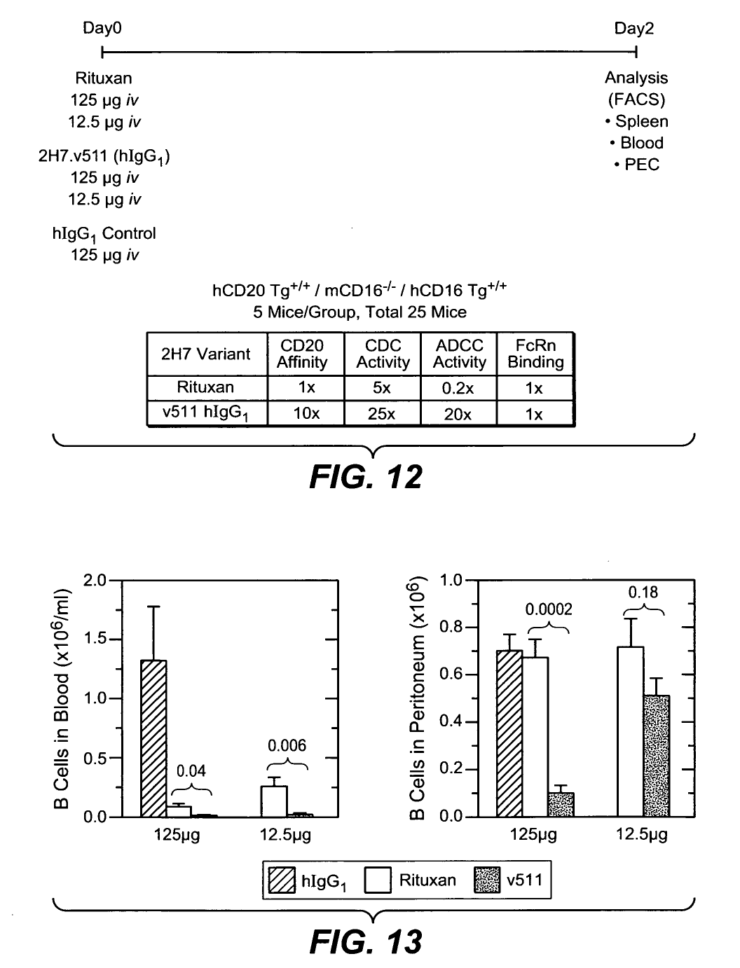

CDC activity was also evaluated as described above, using WIL2-S cells and human complement. The results, shown in FIG. 5, indicated increased CDC potency for 2H7.v51 as compared to 2H7.v588, and greatly increased potency compared to 2H7.v16. As observed with previously described variants, enhanced CD20 affinity appears to enhance CDC activity in vitro. In a separate CDC assay comparing 2H7.v511 with other 2H7 variants, v511 also exhibited increased CDC activity over v31, v488 and v114, as shown in FIG. 10. The CDC assay in FIG. 10 was performed in a 96 microwell plate format essentially as described above, with modifications as follows. 50 ul of serially diluted 2H7.v511 (and Rituxan in experiments summarized in FIG. 12 in example 15), starting at 300-1000 nM, was incubated with 50 ul of normal B cells (1×10⁶/ml, 50,000 cells per well) or 50 μL of WIL2-S cells (30,000) along with 50 μL of a 1:4 dilution of normal human serum complement (Quidel). Normal B cells were isolated from whole blood drawn in heparin, using negative selection (Rosettesep, StemCell Technologies), and Ficoll-Paque Plus (Amersham Biosciences) gradient separation. After a 2 hour incubation at 37° C., 50 uL of Alamar Blue (Biosource International) was added and incubated for an additional 18 hours at 37° C. The plates were briefly shaken for 15 minutes and then read on a florescent plate reader (Ext. 535, Emt 595) to determine the relative florescent units (RFU). The RFU value observed was plotted relative to concentration of mAb in KaleidaGraph and curves were drawn using a 4 parameter fit.

### Example 15

### B Cell Depletion In Vivo

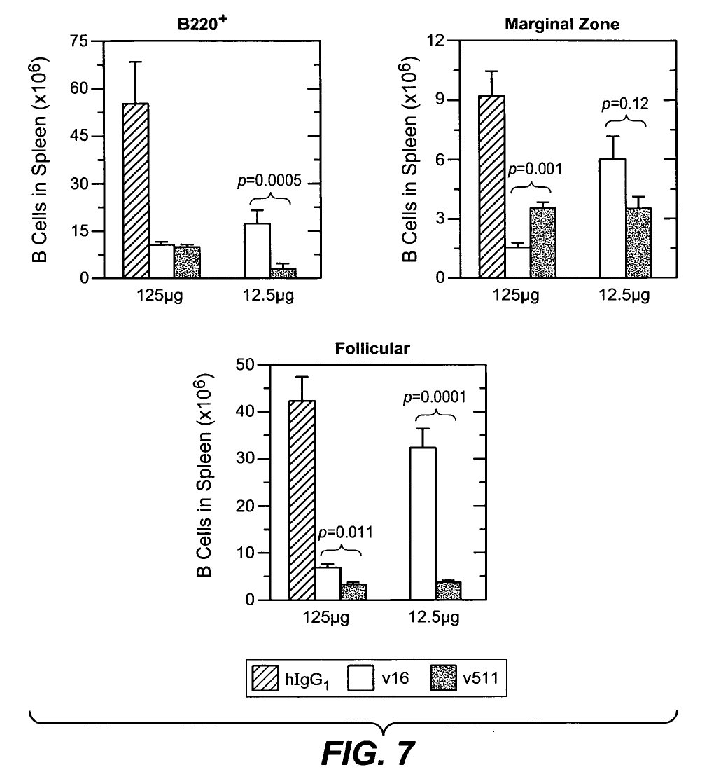

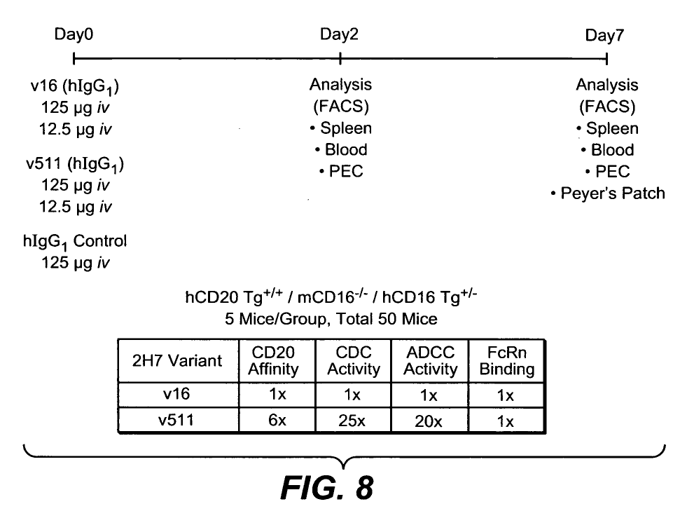

The in vivo activity of 2H7.v511 was compared with that of 2H7.v16 in a transgenic mouse model of B-cell depletion. Transgenic mice (hCD20 Tg^{+/-} mCD16^{-/-} hCD16 Tg^{+/+}), n=5/group, were injected intravenously with 125 ug or 12.5 ug (equivalent to 5 and 0.5 mg/kg) of antibody per mouse. hIgG1 (open bar) in each of FIGS. 6, 7, 13, and 14 is an isotype control antibody. At day two, the number of B-cells in the blood and selected tissue compartments was counted by FACS analysis. The results (FIG. 6) showed that 2H7.v511 more effectively depleted B-cells in the blood and peritoneal cavity than 2H7.v 16 at both low and high doses. In the spleen (FIG. 7), 2H7.v511 showed slightly to significantly greater depletion at the low dose, but this difference was not seen at the high dose. FIG. 8 shows a schematic of the experiment and summarizes the levels of CD20 binding and Fc function between v16 and v511.

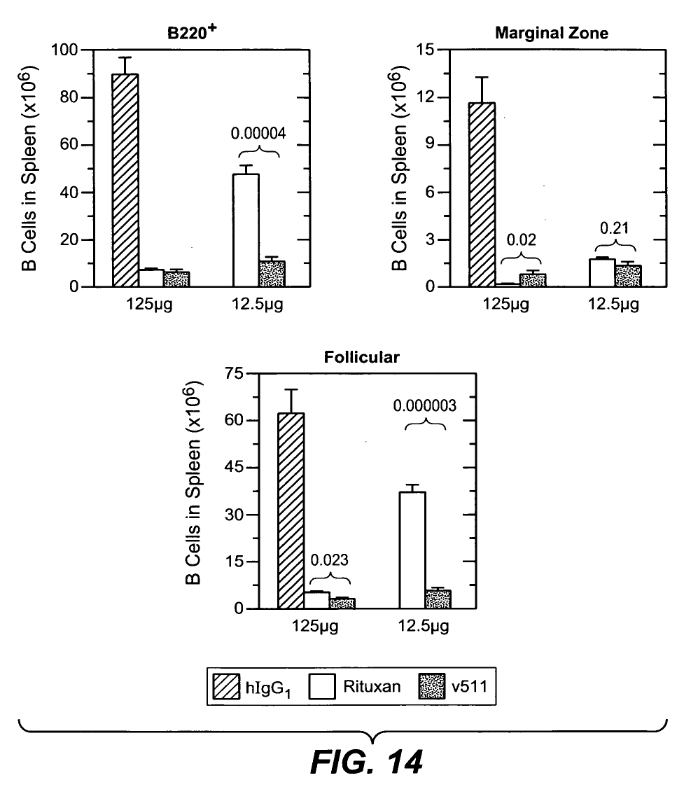

2H7.v511 B cell depletion was also compared to rituximab (Rituxan®) in the mouse model; the experiment is outlined in FIG. 12. As shown in FIG. 13, 2H7.v511 showed greater B-cell depletion in the blood and peritoneal cavity than rituximab at both low and high doses. FIG. 14 shows the B cell depletion in the spleen. At the low dose of 0.5 mg/kg (12.5 ug in the figures), total and follicular splenic B-cell depletion was more effective with v511 than with rituximab; however, v511 appeared similar to or slightly less effective than rituximab in mediating marginal zone B-cell depletion.

In vivo effects of 2H7.v511 are also studied in cynomolgus monkeys essentially as described in Example 9 above.

### Example 16

### In Vivo Suppression of Tumor Growth

The ability of 2H7.v511 to inhibit the growth of the Raji human B-cells, a lymphoma cell line (ATCC CCL 86), is evaluated in Balb/c nude (athymic) mice following the procedure as described in Example 10 above.

### Example 17

### In Vivo Studies of FcRn Binding Effects on Pharmacokinetics

To determine the effects of improved FcRn binding on the pharmacokinetics of these Fc variant antibodies in vivo, cynomolgus (Macaca fascicularis) monkeys or other primate species are injected intravenously with each antibody variant, and blood samples collected over time to monitor the clearance of the antibody. Several animals are injected at one or more dose levels. In one experiment, a single i.v. dose of 1-20 mg/kg is injected at time 0 on day 1. Blood (serum) samples are collected from each animal prior to dosing and at 6 h, 24 h, and 72 h after dosing. Additional samples are collected on day 8, day 10, day 30, and day 60. The concentrations of antibody in the serum samples are determined using an ELISA. The time-dependent decrease in antibody concentration in the serum is modeled using standard pharmacology techniques (Shargel and Yu, Applied Pharmaceutics and Pharmacokinetics, Fourth edition, pp. 67-98, Appleton and Lange, Stamford, Conn. (1999)). A two-compartment model is used to account for the initial distribution of antibody to the tissues (alpha phase), followed by a terminal or elimination phase (beta phase). The elimination half-life (t_{1/2}^{β}) so calculated reveals effects of improved FcRn binding because FcRn functions to maintain IgG in the circulation.

### Example 18

### ADCC, CDC Activity with Cells from CLL Patients

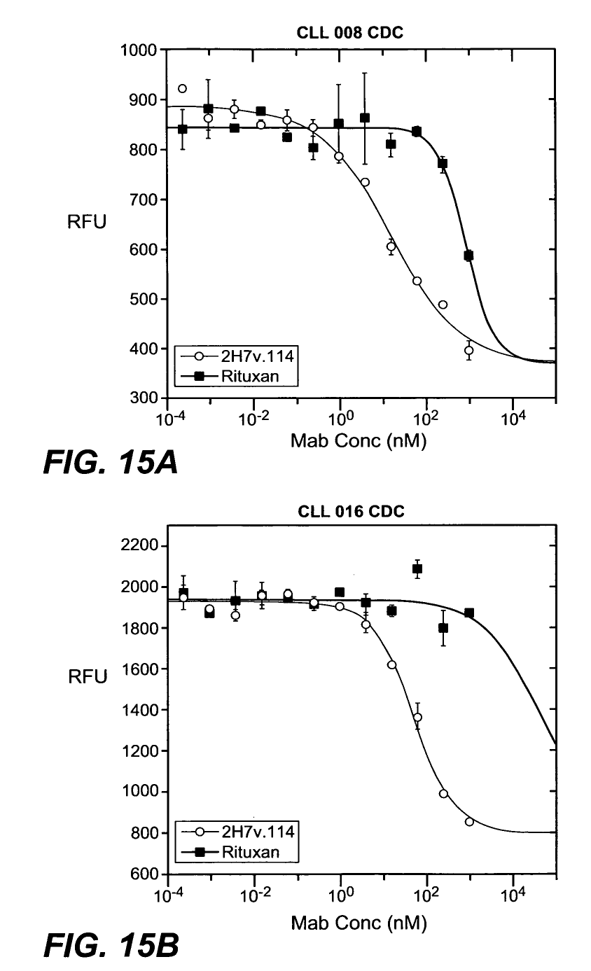

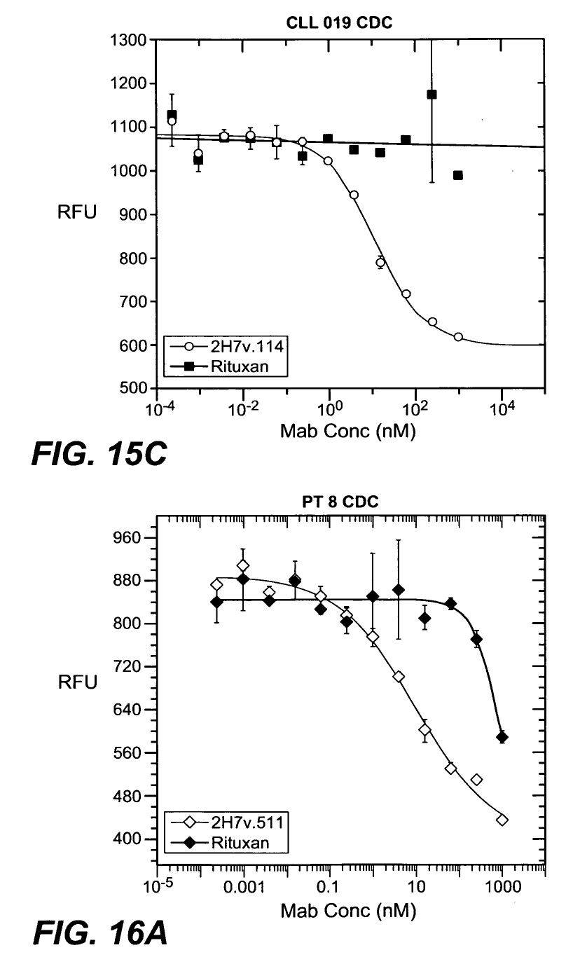

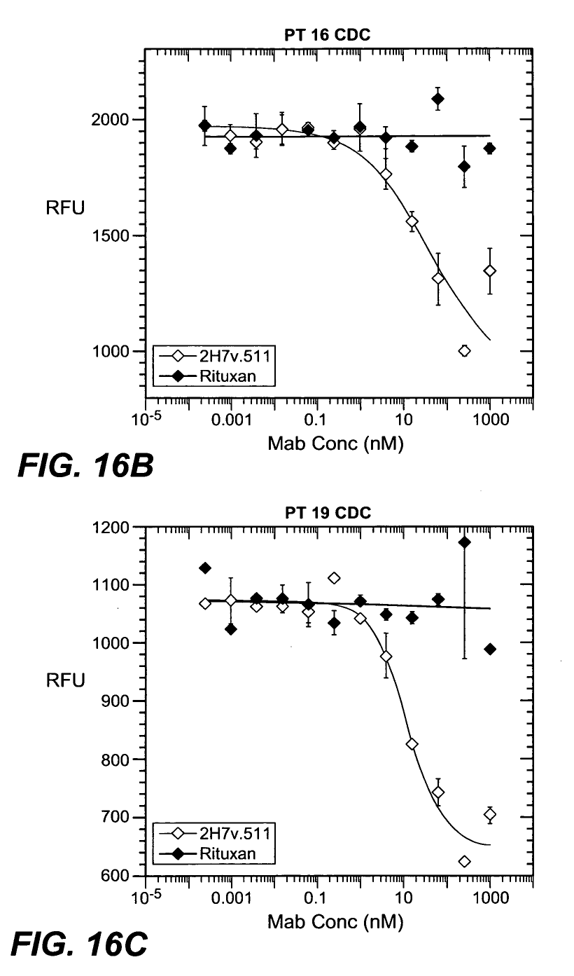

In this study, the complement activities of antibody 2H7.v511 and v114 on B cells from CLL patients were tested. PBMCs were isolated from three CD20 positive, CLL patients (in CLL patients, most of cells found in the PBMC fraction are B cells). In a 96 well microwell format, 50,000 PBMCs from each patient were mixed with 50 ul titrated mAb (v114, v511, or Rituxan™) starting from 1000 nM and doing 1:3 fold serial dilutions of the antibody. Each well received 50 ul of normal human serum complement which had been diluted 1:4. The mixture was incubated for 2 hours at 37° C., then 50 ul of metabolic dye indicator Alamar Blue (indicator of lysis) was added and incubation continued at 37° C. overnight (˜16 hrs). The 96-well plate was shaken at room temperature for 1 minute and read on flourescent plate at 535 nm/590 nm. Relative fluorescent units versus antibody concentration was plotted to obtain EC50. From the results shown in FIG. 15 comparing v114 and Rituxan in CDC acitivity, and FIG. 16 comparing v511 and Rituxan, both v511 and v114 for all 3 patients are about equivalent in potency, but significantly better than Rituxan and v.16.

In a separate study, the ADCC potency of 2H7v.511 and Rituxan with the MEC1 B-CLL cell line (Stacchini, A et al. (1999) Leukemia Res. 23, 127-136) and B cells from B-CLL (B cell related CLL) patients in vitro assays was compared. The experiments were performed as follows. NK cells are isolated from 100 mL of normal human whole blood using negative selection following the manufacture's (RosetteSep, StemCell Technologies) recommended protocol. Whole blood donors vary in the phenotypic expression of C16 on NK cells. The allelic differences occur at position 158 and include valine-valine, valine-phenyalanine, and phenyalanine-phenyalanine phenotypes. Typically, assays were performed with the valine-phenyalanine phenotype. Assays were done in round bottom 96 microwell plates as follows. 50 uL of serially diluted amounts of mAbs, starting at 10-100 nm, was incubated with 50 uL of target cells. Target cells include the B-CLL MEC1 cell line (10,000 per 50 uL) and also PBMCs (30,000 per 50 uL) from B-CLL patients. PBMC were isolated from the whole blood of CLL donors using standard Ficoll-Paque Plus gradient separation. After a 30 minute room temperature incubation with the serially diluted antibodies, 50 uL of NK cells (50,000) were added and incubated for an additional 4 hours at 37° C. The plate were centrifuged at 1500 rpm for 10 minutes and 100 uL of the cell media was removed. The level of cell lysis was determined by measuring the amount of lactate dehyrogenase (LDH kit Roche) released from lysed cells. The percent lysis relative to mAb concentration was determined and plotted in KaleidaGraph using a 4-parameter curve fit. IC₅₀ values were reported for the MEC1 cell line. Point to point line graphs were used to plot CLL PBMC data and the percent killing at 30, 5, and 0.8 nM was reported

The results showed that the ADCC activity of 2H7v.511 was higher than Rituxan in all three patient samples. At the 30 nM, 5 nM and 0.8 nM of antibody, the improvement of 2H7v.511 over Rituxan was 2.8-fold, 6.2-fold and 5.4-fold respectively. NK cells were of the VF phenotype.

The ADCC activity of 2H7v.511 was also higher than Rituxan mediating cell lysis of MEC1 cells. 2H7v.511 had greater than 13 fold higher activity with the IC₅₀ of 2H7v.511 at 0.95 pM and Rituxan at 0.012 nM. NK cells were of the VV phenotype.

### CONCLUSION

In summary, 2H7.v511 and v114 show increased CD20 binding affinity, increased CDC activity, increased ADCC activity, and increased in vivo potency for B-cell depletion in vivo as compared with 2H7.v16.

### Example 19

A study was performed in cynomolgus monkeys with 2H7.v511 and v 114 according to the study design shown in the table below to evaluate safety, PK in primates, and in vivo normal B cell depletion. The animals received 4 weekly doses at each dose level. Analysis of the animals at either the 2.5 or 50 mg/kg×4 dosing showed peripheral B cell depletion with both v511 and v114. In the pilot study with v511, at 50 mg/kg the peripheral B cells were rapidly and completely depleted. Based on necropsy findings, tissue B cells were also significantly depleted at this dose.

| | | | |
|---|---|---|---|
| | | Dose | Number |
| Group | Number of | Level | Euthanizaed: |

| No. | Males/Females | (mg/kg) | Day 29 | Day 254 |
|---|---|---|---|---|
| 1 | 4/4 | 0 (control) | 2/2 | 2/2 |
| 2 | 4/4 | 2.5 | 2/2 | 2/2 |
| 3 | 4/4 | 5 | 2/2 | 2/2 |
| 4 | 4/4 | 10 | 2/2 | 2/2 |
| 5 | 4/4 | 25 | 2/2 | 2/2 |
| 6 | 4/4 | 50 | 2/2 | 2/2 |

### Example 20

### Phase I/II Clinical Study for NHL

The table below shows one Phase I/II clinical study design for treating Non-Hodgkin's Lymphoma (NHL) using antibody hu2H7.v16 in one study and hu2H7.v511 in a parallel study. The antibody is administered via intravenous infusion. This dosing regimen can also be used with other hu2H7 variants of the invention described above.

2H7 Phase I/II Study for Non-Hodgkin's Lymphoma - Dose/Infusion Rate

| Cohort | Week 1 | Week 2 | Week 3 | Week 4 | Total Dose | |
|---|---|---|---|---|---|---|
| A | 100 mg/m2 | 100 mg/m2 | 100 mg/m2 | 100 mg/m2 | 400 | mg/m2 |
| | Rituxan-like | Rituxan-like | Rituxan-like | Rituxan-like | | |
| | infusion | infusion | infusion | infusion | | |
| B | 250 mg/m2 | 250 mg/m2 | 250 mg/m2 | 250 mg/m2 | 1000 | mg/m2 |
| | Rituxan-like | Rituxan-like | Rituxan-like | Rituxan-like | | |
| | infusion | infusion | infusion | infusion | | |
| C | 375 mg/m2 | 375 mg/m2 | 375 mg/m2 | 375 mg/m2 | 1500 | mg/m2 |
| | Rituxan-like | Rituxan-like | Rituxan-like | Rituxan-like | | |
| | infusion | infusion | infusion | infusion | | |
| D | 375 mg/m2 | 375 mg/m2 | 375 mg/m2 | 375 mg/m2 | 1500 | mg/m2 |
| | Rituxan-like | Accelerated | Accelerated | Accelerated | | |
| | infusion | infusion | infusion | infusion | | |
| E | 100 or 250 mg/m2 | 375 mg/m2 | 375 mg/m2 | 375 mg/m2 | 1225 or 1375 | mg/m2 |
| | Rituxan-like | Accelerated | Accelerated | Accelerated | | |
| | infusion | infusion | infusion | infusion | | |

### Example 21

### Phase I Clinical Study 2 for NHL

The table below shows another Phase I clinical study design for treating Non-Hodgkin's Lymphoma (NHL) using antibody hu2H7.v16 in one study and hu2H7.v511 in a parallel study. The antibody is administered via intravenous infusion. This dosing regimen can also be used with other hu2H7 variants of the invention described above. Each dose is give 3 weeks apart for a total of 8 doses.

3 cohorts (10 patients/cohort) q3wksx8

| Cohort | | | | | | | | | |
|---|---|---|---|---|---|---|---|---|---|
| (Total dose) | Dose 1 | | | | | | | | |
| mg/m2 | (mg/m2) | Dose 2 | Dose 3 | Dose 3 | Dose 4 | Dose 5 | Dose 6 | Dose 7 | Dose 8 |
| A | 200 | 200 | 200 | 200 | 200 | 200 | 200 | 200 | 200 |
| 1600 | | | | | | | | | |
| B | 375 | 375 | 375 | 375 | 375 | 375 | 375 | 375 | 375 |
| 3000 | | | | | | | | | |
| C | 375 | 750 | 750 | 750 | 750 | 750 | 750 | 750 | 750 |
| 5625 | | | | | | | | | |

### Example 22

### Phase I/II Dose Escalation Cohorts for NHL

The Table below shows a Phase I/II clinical study design for treating Non-Hodgkin's Lymphoma (NHL) using antibody hu2H7.v511. Patients are dosed weekly×4 weeks. Efficacy is measured, e.g., by tumor shrinkage by CT scan.

(3-6 patients per cohort)

| | | | | | Total |
|---|---|---|---|---|---|
| | Week 1 | Week 2 | Week 3 | Week 4 | Dose |
| A | 12.5 mg/m2 | 12.5 mg/m2 | 12.5 mg/m2 | 12.5 mg/m2 | 50 |
| | | | | | mg/m2 |
| B | 25 mg/m2 | 25 mg/m2 | 25 mg/m2 | 25 mg/m2 | 100 |
| | | | | | mg/m2 |
| C | 50 mg/m2 | 50 mg/m2 | 50 mg/m2 | 50 mg/m2 | 200 |
| | | | | | mg/m2 |
| D | 100 mg/m2 | 100 mg/m2 | 100 mg/m2 | 100 mg/m2 | 400 |
| | | | | | mg/m2 |
| E | 200 mg/m2 | 200 mg/m2 | 200 mg/m2 | 200 mg/m2 | 800 |
| | | | | | mg/m2 |
| F | 400 mg/m2 | 400 mg/m2 | 400 mg/m2 | 400 mg/m2 | 1600 |
| | | | | | mg/m2 |
| G | 800 mg/m2 | 800 mg/m2 | 800 mg/m2 | 800 mg/m2 | 3200 |
| | | | | | mg/m2 |

### Example 23

### Phase I/II Dose Escalation Cohorts for NHL

The Table below shows another Phase I/II clinical study design for treating Non-Hodgkin's Lymphoma (NHL) using antibody hu2H7.v511. Patients are dosed weekly×4 weeks. Efficacy is measured, e.g., by tumor shrinkage by CT scan.

| | | | | | Total |
|---|---|---|---|---|---|
| | Week 1 | Week 2 | Week 3 | Week 4 | Dose |
| A | 25 mg/m² | 25 mg/m² | 25 mg/m² | 25 mg/m² | 100 |
| | | | | | mg/m² |
| B | 25 mg/m² | 50 mg/m² | 50 mg/m² | 50 mg/m² | 175 |
| | | | | | mg/m² |
| C | 50 mg/m² | 100 mg/m² | 100 mg/m² | 100 mg/m² | 350 |
| | | | | | mg/m² |
| D | 100 mg/m² | 200 mg/m² | 200 mg/m² | 200 mg/m² | 700 |
| | | | | | mg/m² |
| E | 200 mg/m² | 400 mg/m² | 400 mg/m² | 400 mg/m² | 1,400 |
| | | | | | mg/m² |
| F | 400 mg/m² | 800 mg/m² | 800 mg/m² | 800 mg/m² | 2,800 |
| | | | | | mg/m² |

### Example 24

### Phase I/II Study of 2H7.v511 in Moderate to Severe Rheumatoid Arthritis Design of Study Treatment

Cohorts of subjects will received two IV infusions of hu2H7.v511 or placebo equivalent at the indicated dose on Days 1 and 15 according to the following escalation plan:

10 mg hu2H7.v511 or placebo equivalent: 8 subjects active drug, 2 control

50 mg hu2H7.v511 or placebo equivalent: 8 subjects active drug, 2 control

100 mg hu2H7.v511 or placebo equivalent: 8 subjects active drug, 2 control

200 mg hu2H7.v511 or placebo equivalent: 8 subjects active drug, 2 control

300 mg hu2H7.v511 or placebo equivalent: 8 subjects active drug, 2 control

500 mg hu2H7.v511 or placebo equivalent: 8 subjects active drug, 2 control

Efficacy

The efficacy of 2H7.v511 will be measured by ACR responses. The percentage of subjects who achieve an ACR20, ACR50, and ACR70 response will be summarized by treatment group and 95% confidence intervals will be generated for each group.

### REFERENCES

References cited within this application, including patents, published applications and other publications, are hereby incorporated by reference.

The practice of the present invention will employ, unless otherwise indicated, conventional techniques of molecular biology and the like, which are within the skill of the art. Such techniques are explained fully in the literature. See e.g., Molecular Cloninig: A Laboratory Manual, (J. Sambrook et al., Cold Spring Harbor Laboratory, Cold Spring Harbor, N.Y., 1989); Current Protocols in Molecular Biology (F. Ausubel et al., eds., 1987 updated); Essential Molecular Biology (T. Brown ed., IRL Press 1991); Gene Expression Technology (Goeddel ed., Academic Press 1991); Methods for Cloning and Analysis of Eukaryotic Genes (A. Bothwell et al. eds., Bartlett Publ. 1990); Gene Transfer and Expression (M. Kriegler, Stockton Press 1990); Recombinant DNA Methodology II (R. Wu et al. eds., Academic Press 1995); PCR: A Practical Approach (M. McPherson et al., IRL Press at Oxford University Press 1991); Oligonucleotide Synthesis (M. Gait ed., 1984); Cell Culture for Biochemists (R. Adams ed., Elsevier Science Publishers 1990); Gene Transfer Vectors for Mammalian Cells (J. Miller & M. Calos eds., 1987); Mammalian Cell Biotechnology (M. Butler ed., 1991); Animal Cell Culture (J. Pollard et al. eds., Humana Press 1990); Culture of Animal Cells, 2^{nd} Ed. (R. Freshney et al. eds., Alan R. Liss 1987); Flow Cytometry and Sorting (M. Melamed et al. eds., Wiley-Liss 1990); the series Methods in Enzymology (Academic Press, Inc.); Wirth M. and Hauser H. (1993); Immunochemistry in Practice, 3rd edition, A. Johnstone & R. Thorpe, Blackwell Science, Cambridge, Mass., 1996; Techniques in Immunocytochemistry, (G. Bullock & P. Petrusz eds., Academic Press 1982, 1983, 1985, 1989); Handbook of Experimental Immunology, (D. Weir & C. Blackwell, eds.); Current Protocols in Immunology (J. Coligan et al. eds. 1991); Immunoassay (E. P. Diamandis & T. K. Christopoulos, eds., Academic Press, Inc., 1996); Goding (1986) Monoclonal Antibodies: Principles and Practice (2d ed) Academic Press, New York; Ed Harlow and David Lane, Antibodies A laboratory Manual, Cold Spring Harbor Laboratory, Cold Spring Harbor, N.Y., 1988; Antibody Engineering, 2^{nd} edition (C. Borrebaeck, ed., Oxford University Press, 1995); and the series Annual Review of Immunology; the series Advances in Immunology.

1. A humanized 2H7 antibody that binds human CD20, or an antigen-binding fragment thereof, the antibody comprising the L chain Variable region (V_{L}) sequence of SEQ ID NO. 25 and the H chain Variable region (V_{H}) sequence of SEQ ID NO. 8 but with amino acid substitution of D56A in VH-CDR2, and N100 in VH-CDR3 is substituted with Y or W.

2. The antibody of claim 1, wherein N100 is substituted with Y.

3. The antibody of claim 1, wherein the N100 is substituted with W.

4. The antibody of claim 1, further comprising the substitution S100aR in VH-CDR3.

5. The antibody of claim 4, further comprising at least one amino acid substitution in the IgG Fc region that improves ADCC and/or CDC activity.

6. The antibody of claim 5, comprising an IgG1 Fc comprising the amino acid substitutions S298A, E333A, K334A, K326A.

7. The antibody of claim 4, further comprising at least one amino acid substitution in the Fc region that improves ADCC but decreases CDC activity.

8. The antibody of claim 7, comprising at least the amino acid substitution K322A.

9. The antibody of claim 8, further comprising the amino acid substitutions S298A, E333A, K334A.

10. The antibody of claim 6, comprising the light chain sequence of SEQ ID NO.26 and heavy chain sequence of SEQ ID NO. 34.

11. The antibody of claim 4, which binds human CD20 with at least 3 fold increased affinity relative to antibody 2H7.v16.

12. The antibody of claim 4, which binds human CD20 with at least 6 fold increased affinity relative to antibody 2H7.v16.

13. The antibody of claim 6, which exhibits at least 20 fold greater antibody dependent cellular cytotoxicity (ADCC) than 2H7.v16.

14. The antibody of claim 6, which exhibits at least 25 fold greater complement cytotoxicity than 2H7.v16.

15. The antibody of claim 1, conjugated to a cytotoxic agent.

16. The antibody of claim 15, wherein the cytotoxic agent is a radioactive isotope or a toxin.

17. The antibody of any of claim 1, which antibody is produced in CHO cells.

18. An isolated nucleic acid that encodes the antibody of claim 1.

19. The nucleic acid of claim 18 which is an expression vector

20. A host cell comprising the nucleic acid of claim 18.

21. (canceled)

22. The host cell of claim 20 that produces the antibody comprising the light chain sequence of SEQ ID NO.26 and heavy chain sequence of SEQ ID NO. 34.

23. The host cell of claim 22 which is a CHO cell.

24. A method of producing the antibody of claim 10, comprising culturing the host cell of claim 22 and recovering the antibody from the cell culture.

25. A composition comprising the antibody of claim 1 and a carrier.

26. The composition of claim 25 comprising the antibody of claim 10 and a pharmaceutically acceptable carrier.

27. An article of manufacture comprising a container and a composition contained therein, wherein the composition comprises an antibody of claim 10.

28. The article of manufacture of claim 27, further comprising a package insert indicating that the composition is used to treat non-Hodgkin's lymphoma.

29. The article of manufacture of claim 27, further comprising a package insert indicating that the composition is used to treat rheumatoid arthritis.

30. A method of treating a CD20 positive cancer, comprising administering to a patient having the cancer, a therapeutically effective amount of the humanized 2H7 antibody of claim 10.

31. The method of claim 30 wherein the CD20 positive cancer is a B cell lymphoma or leukemia.

32. The method of claim 31 wherein CD20 positive cancer is non-Hodgkin's lymphoma (NHL).

33. The method of claim 32 wherein the cancer is chronic lymphocytic leukemia (CLL) or SLL.

34. (canceled)

35. The method of claim 31, wherein the antibody is administered via intravenous infusion.

36. The method of claim 35, wherein the antibody is administered at a dosage in the range of about 100 mg/m² to 375 mg/m² per dose.

37. The method of claim 36, wherein the antibody is administered at a dosage of 375 mg/m² per dose weekly for at least 4 doses in the treatment of non-Hodgkin's lymphoma.

38. The method of claim 30, further comprising administering to the patient at least one chemotherapeutic agent.

39. The method of claim 38, wherein the cancer is non-Hodgkin's lymphoma (NHL) and the chemotherapeutic agent is selected from the group consisting of doxorubicin, cyclophosphamide, vincristine and prednisolone.

40. A method of alleviating an autoimmune disease, comprising administering to a patient suffering from the autoimmune disease, a therapeutically effective amount of the humanized 2H7 antibody of any one of claims 6-10.

41. The method of claim 40, wherein the antibody comprises the light and heavy chain amino acid sequence of SEQ ID NO. 26 and 34, respectively.

42. The method of claim 41, wherein the autoimmune disease is selected from the group consisting of rheumatoid arthritis and juvenile rheumatoid arthritis, systemic lupus erythematosus (SLE) including lupus nephritis, Wegener's disease, inflammatory bowel disease, ulcerative colitis, idiopathic thrombocytopenic purpura (ITP), thrombotic throbocytopenic purpura (TTP), autoimmune thrombocytopenia, multiple sclerosis, psoriasis, IgA nephropathy, IgM polyneuropathies, myasthenia gravis, ANCA associated vasculitis, diabetes mellitus, Reynaud's syndrome, Sjogren's syndrome, Neuromyelitis Optica (NMO) and glomerulonephritis.

43. The method of claim 42, wherein the autoimmune disease is rheumatoid arthritis.

44. The method of claim 43, further comprising administering to the patient a second therapeutic agent.

45. The method of claim 44, wherein the second therapeutic agent is an immunosuppressive agent.

46. The method of claim 45, wherein the immunosuppressive agent is methotrexate.

47. The method of claim 43, wherein the antibody is administered intravenously or subcutaneously.

48. The method of claim 43, wherein the antibody is administered intravenously at a dosage in the range of 10 mg to 500 mg per dose.

49. The method of claim 48, wherein the antibody is administered at a dosage of 200 mg/dose for at least two doses.

50. The method of claim 40, further comprising administering to the patient a second therapeutic agent.

51. The method of claim 50, wherein the second therapeutic agent is a BAFF antagonist.

52. The method of claim 51, wherein the BAFF antagonist is an anti-BR3 antibody or a BR3-Fc fusion protein.

53. The method of claim 30, further comprising administering to the patient, a VEGF antagonist.

54. A liquid formulation comprising humanized 2H7.v511 antibody at about 20 mg/ml, in 20 mM sodium acetate, pH 5.5, 4% trehalose dihydrate, 0.02% polysorbate 20, for intravenous administration.

55. A liquid formulation comprising humanized 2H7.v114 antibody at about 20 mg/ml, in 20 mM sodium acetate, pH 5.3, 240 mM (8%) trehalose dihydrate, 0.02% Polysorbate 20.

56. A liquid formulation comprising humanized 2H7.v16 antibody at about 30 mg/ml in 20 mM sodium acetate, pH 5.3, 4% trehalose dehydrate, 0.02% polysorbate 20, for intravenous administration.

57. The method of claim 36, wherein the antibody is administered in weekly doses of 200 mg/m² per dose, for at least 4 weeks in the treatment of non-Hodgkin's lymphoma.

58. The method of claim 48, wherein the antibody is administered at a dosage of 500 mg/dose for at least two doses.
